(19) United States
(12) Patent Application Publication
(10) Pub. No.: US 2025/0276180 A1
(43) Pub. Date: Sep. 4, 2025

(54) SYSTEMS AND METHODS FOR TREATING MOOD DISORDERS

(71) Applicant: Inner Cosmos Inc., Scotts Valley, CA (US)

(72) Inventors: Eric Claude LEUTHARDT, St. Louis, MO (US); Daniel W. MORAN, St. Louis, MO (US); Meron GRIBETZ, New York City, NY (US)

(73) Assignee: Inner Cosmos Inc., Scotts Valley, CA (US)

(21) Appl. No.: 19/211,325

(22) Filed: May 19, 2025

Related U.S. Application Data
(63) Continuation of application No. 17/253,176, filed on Dec. 17, 2020, now abandoned.
(60) Provisional application No. 62/687,264, filed on Jun. 20, 2018.

(51) Int. Cl. A61N 1/36; A61N 1/372

(52) U.S. Cl. CPC A61N 1/36096; A61N 1/36132; A61N 1/36139; A61N 1/37211

(57) ABSTRACT

Systems for treating a mood disorder in a patient includes implantable device(s) including one or more electrodes for sensing cortical signals and for stimulating one or more brain regions. Processor/controller(s) in communication with the electrode(s) receive and process cortical signals from electrode(s) and control the stimulating of brain region(s). The system includes portable communication device(s) operable by the patient and having software for acquiring ecological mood assessment (EMA) data representative of the patient's mood and communicating the EMA data to the processor/controller(s) and/or to at least one remote processor. Sensors may also be used to record patient data. The data is processed by the processor/controller(s), and/or by a processor of the portable communication device and/or by the remote processor(s) for modulating and/or controlling the stimulation the brain region(s) to treat the mood disorder. The implantable device(s) may include a power source. The implantable device(s) may be implanted intra-cranially and/or intra-calvarially.

### RELATED APPLICATIONS

This application is a Continuation of U.S. patent application Ser. No. 17/253,176 filed on Dec. 17, 2020, which is a National Phase of PCT Patent Application No.

PCT/IB2019/055217 having International Filing Date of Jun. 20, 2019, which claims the benefit of priority under 35 USC § 119(e) of U.S. Provisional Patent Application No. 62/687,264 filed on Jun. 20, 2018. The contents of the above applications are all incorporated by reference as if fully set forth herein in their entirety.

### FIELD AND BACKGROUND OF THE INVENTION

The present invention, in some embodiments thereof, relates to the field of systems and methods for treatment of mood disorders and more specifically to brain computer interface (BCI) systems and methods for treating depression.

Current antidepressant therapies have substantial limitation in effectively controlling symptoms associated with depression. There are four million Americans who are diagnosed with recurrent and or severe treatment-resistant form of depression known as refractory major depressive disorder. Subjective diagnostics, varied manifestations of the disorder, and antidepressant treatments with limited theoretical bases each contribute to the limitations of therapeutic efficacy and differing levels of treatment resistance in the refractory population. Stimulation-based therapies for these treatment-resistant patients are plagued with inconsistent reports of efficacy and variable side effects. Many of these problems stem from the unknown mechanisms of depressive disorder pathogenesis, which prevents the development of treatments that target the specific underlying causes of the disorder. Other problems likely arise due to the non-specific stimulation of various limbic and paralimbic structures in an open-loop configuration. Closed-loop neurostimulation device designs have been proposed but the lack of effective and validated biomarkers have hampered the ability of these systems to deliver appropriate and timely stimulation regimes.

Depression is one of the top causes of mortality and sub-standard daily functioning in North America Wells et al., 1989). The term “depression” is currently used to describe a broad set of disparate pathologies sharing a common set of symptoms—pathologies that manifest as abnormal control and expression of mood and emotion (Davidson et al., 2002). Depressed individuals have a diversity of clinical symptomatology. This can include a dispirited mood, a reduced enjoyment with routine tasks, a distorted sleep schedule, altered behavior/appetite/weight, a change in motor kinetics, a decreased energy level, impaired focus, thoughts of worthlessness or guilt, and thoughts of death or suicide over an extended period of time (First and Ross, 2000; Kroenke et al., 2001). Current treatment measures do not always effectively control symptoms in many depressed patients, especially those with refractory major depressive disorder (refractory MDD) (Kessler et al., 2005; Cyberonics, 2007). Refractory MDD is characterized by recurrent, long-lasting cycles of severe, often suicidal depressive episodes that do not remit using multiple types of antidepressant therapies. A depressive episode persists for up to a year (Judd et al., 1998), significantly impairing the health, activities, work, and well being of the affected patient (Manji et al., 2001). Even with the optimal FDA-approved antidepressant treatments a substantial percentage of MDD patients will have recurrent episodes (Mueller et al., 1999; Kessler et al., 2003). It is clear that treatments that are more efficacious, reliable, personalized and durable are needed.

Currently, 50-60% of all depressed patients remain partially or fully unresponsive to a first course of properly prescribed therapy (Fava, 2003). Up to 20% of these patients require more extreme treatment measures, employing multiple antidepressant medications and/or electroconvulsive therapy (ECT) with variable success rates (Fava, 2003; Mayberg et al., 2005). A meta-analysis of 74 published and unpublished antidepressant efficacy trials, involving 12 antidepressant drugs and 12,564 participants, showed that only 51% of the trials whose data was submitted to the FDA ended with positive results (Turner et al., 2008). A separate meta-analysis of 47 published and unpublished FDA clinical trial datasets from selective-serotonin reuptake inhibitor (SSRI) efficacy trials showed that six of the seven most-prescribed SSRI antidepressant drugs available within the last 25 years only show clinically significant benefits over placebo for “the upper end of the very severely depressed category” (Kirsch et al., 2008). The latter study suggests that SSRIs, which are most often the first class of prescribed drugs for depressive disorders, pose greater risks to patient health than benefits for symptom relief in the majority of patients (Kirsch et al., 2008; Turner et al., 2008).

More recently, stimulation-based technologies, which are designed to electrically modulate abnormal neural activity, are emerging as potential therapeutic approaches for refractory MDD patients. The challenge, however, remains that the efficacy of these technologies are hampered by an incomplete understanding of the pathophysiology of depressive disorders and a lack of reproducible and quantifiable biological markers (i.e., biomarkers) of depressed states (antidepressant treatment response is still subjectively evaluated using patient-reported symptom relief, effectively ignoring the prospect of using objectively-quantified, depression-linked biomarker levels to quantify antidepressant responses and to optimize treatment). To date, there have been numerous structural, functional, and genetic abnormalities associated with depression which have been identified. Discoveries in the epilepsy research field sparked interests in closed-loop neuroprostheses, where biological indicators of an impending seizure are used to determine the time at which an electrical or chemical stimulus must be applied to stop a seizure (Dumitriu et al., 2008). This process, known as responsive neurostimulation, is unique to closed-loop devices. It is intended to replace continuous or periodic open-loop stimulation designs so that tailored therapy, based on quantifiable symptom-linked biomarker abnormalities, is provided in a dose-dependent manner only when it is necessary (Sun et al., 2008; Goodman and Insel, 2009). It has been posited that a similar approach can also be taken for depression. To date, however, despite the recent advancements in depression research, no closed-loop prosthesis exists for treating refractory MDD. This is in large part due to the lack of candidate quantifiable biological markers of depression that operate in a timescale that can meaningfully inform a brain stimulator. While seizures can be reasonably detected with an implanted recording system, there is only limited evidence that similar signals can be identified for active states of depression. This is in part due to lack of scientific insights into the fundamental mechanisms of depression and also the high level of individual variability in pathologic causes of the depression.

### Current Diagnostic and Treatment Protocols.

Depression is currently diagnosed through an evaluation of a patient's reported symptoms, clinical history, and full physical examination. A patient is often initially assessed using a depression-specific standardized evaluation such as the nine item Patient Health Questionnaire (PHQ-9), Hamilton Depression Rating Scale (HAM-D or HDRS), or Montgomery-Asberg Depression Rating Scale (MADRS) (Kearns et al., 1982; Kroenke et al., 2001). Each survey is used to estimate the severity of the symptoms used to diagnose depression in accordance with DSM-IV criteria. The patient's clinical history and physical examination are then used to rule out other obvious and treatable explanations for the symptoms Depression Guideline Panel, 1994). Diagnosing refractory MDD is a lengthy process that often is not in the best interest of the patient's health due to potentially life-threatening antidepressant side effects (e.g., violent behavior, cardiovascular problems, and/or recurrent thoughts of death/suicide) (Peretti et al., 2000; Mann, 2005). The most common first line of treatment for an MDD patient is psychotherapy and/or a low-dose SSRI antidepressant therapy. In psychotherapy sessions, a patient is taught to change thinking and behavior patterns in an effort to modulate limbic-cortical pathways in regions of the prefrontal cortex, hippocampus, and cingulate cortex that are associated with normal emotions and behavior (Goldapple et al., 2004). After a recommended 6-12 weeks on a particular antidepressant (Quitkin et al., 1986; Mann, 2005), effectiveness may be assessed using the HAM-D or MADRS questionnaire (Despite the recommended evaluation timeframe, efficacy is typically assessed after 4-6 weeks of treatment). If the patient shows some benefit with zero or non-problematic symptoms, a higher dose of the same medication or a second antidepressant is prescribed. If a patient receives no significant benefit from at least two properly prescribed antidepressants (i.e., correct dose and sufficient evaluation timeframe), he or she is diagnosed with refractory MDD (Dumitriu et al., 2008). The level of treatment resistance is then estimated using one of several non-standardized algorithms, most notably the five stage model put forth by Thase and Rush (1997) (Dumitriu et al., 2008). Objective diagnostic tests based on quantifiable depressive disorder-specific biomarkers are needed to improve diagnostic accuracy and the classifications of differing manifestations of the disorder. In summary, a major contributor to failing depressive disorder treatments stems from the lack of objective diagnostic criteria, which impedes more accurate distinctions among depressed patients who share the same common symptom profile, but develop depressive disorders through differing circumstances (Lacasse and Leo, 2005). Since antidepressant therapies do not have well-defined targets, proven mechanisms of action, and consistent reports of clinical efficacy, it is no surprise that varying levels of treatment resistance are consistently reported (Thase and Rush, 1997; Fava, 2003; Mann, 2005; Belmaker and Agam, 2008; Kirsch et al., 2008). More individually tailored antidepressant therapies, both with regard to the pathology and in the timescale of modification, are needed if enhanced therapeutic efficacies are desired in the refractory population.

BRAIN stimulation for depression treatment.

There are few alternative options for pharmacotherapy in treating depression. In severe cases, electroconvulsive therapy (ECT) is most commonly used over several weeks to help control depressive symptoms. This traditional treatment paradigm for treatment-resistant patients involves non-specific, but noninvasive stimulation of broad regions of the cortex. The patients must be lightly anesthetized and/or sedated and often experience significant adverse side effects (e.g., retrograde amnesia that often does not fully improve over time) (Marangell et al., 2007; Dumitriu et al., 2008). However, despite its inherent limitations, ECT has provided more antidepressant benefit to refractory MDD patients than any other FDA-approved treatment option. In addition to inherent complications associated with the therapy, this approach also is problematic in that it requires significant tertiary medical resources, and thus is not able to fully scale to the large clinical population in need.

Transcranial magnetic stimulation (TMS) was introduced by Barker et al. (1985) (Klein et al., 1999). By noninvasively activating target cortical regions, TMS allows investigators to selectively study brain function in a simplified and relatively safe manner (Figiel et al., 1998; Klein et al., 1999). In the last few decades it has received considerable interest as a therapeutic tool in a variety of neurological disorders, stemming from its favorable spatial selectivity over ECT, noninvasive nature, and generally tolerable side effects (Figiel et al., 1998; Klein et al., 1999; Janicak et al., 2008). As a result, TMS is now used as an FDA-approved treatment option for refractory MDD.

Transcranial magnetic stimulation is typically administered by pulsing a current through a circular or figure-8 coil positioned over the cortical regions of interest. The resulting oriented magnetic field pulses generate an electric field within the superficial layers of cortex (with a maximum depth of 1 cm, Dumitriu et al., 2008), depolarizing neurons when a sufficient electric field is generated (Fitzgerald et al., 2002). Device size limitations preclude the use of this technology in a fully implantable closed-loop neuroprosthesis. Current TMS devices are large and typically only accessible through outpatient procedures (such as NeuroStar® TMS Therapy, Neuronetics, 2009). TMS device size, which is proportional to the size of the stimulated cortical area, is limited by a tradeoff between coil size and the magnitude of current required to generate the same magnetic field in a smaller device (Cohen and Cuffin, 1991). As a result, TMS is not suitable for use in a fully implantable neuroprosthesis unless fundamental design changes are made to considerably decrease device size without sacrificing performance. Here again the need for high level infrastructure for using TMS limits that technology to scale to the population.

There are many subtypes of TMS, classified according to stimulation parameters and mode of application. Two such traditional TMS subtypes are: rapid-rate/repetitive transcranial magnetic stimulation (rTMS) that includes any stimulation paradigm using frequencies >1 Hz) and low-frequency/slow transcranial magnetic stimulation (sTMS) that includes any stimulation paradigm using frequencies <1 Hz). The TMS subtypes produce differing cortical activation properties, depending largely on stimulation parameters, coil shapes and sizes, stimulation sites, and stimulation orientations—and are associated with studies that report conflicting therapeutic efficacies. However, it is believed that rTMS produces more antidepressive effects, as one study of cerebral blood flow showed significant increases in blood supply to prefrontal cortical and limbic regions following rTMS and marked decreases following sTMS (Speer et al., 2000). This variability in effect likely reflects the challenges relative to the variable nature of the neural pathologies that are being treated. Also this type of treatment is open loop and not provided according to any biomarkers or tuned to patients' symptomatology.

Deep brain stimulation (DBS) was first used for treating depression in 1954 (Poole, 1954; Hardesty and Sackeim, 2007). However, DBS gained considerable research interest and momentum in 1987, when Benabid et al. (1987) successfully relieved Parkinsonian tremors in a patient through high-frequency stimulation of one thalamic nucleus ventralis intermedius and removal of the other. Benabid et al.'s paper showed that high-frequency electrical stimulation of a dysfunctional brain structure was as effective as surgically removing the same part of the brain, thereby promoting DBS therapy as a less-invasive and less-extreme alternative to resection surgeries (Benabid et al., 1987; Hardesty and Sackeim, 2007).

The power of DBS in treating refractory psychiatric disorders has become increasingly apparent throughout the last few decades, largely through unexpected side effects observed in non-depressed DBS patients. For example: in an older woman without any known psychiatric disorders (implanted with a deep brain stimulator for Parkinson's disease), high-frequency DBS therapy applied to the left substantia nigra caused temporary suicidal depression that reversed once stimulation ceased (The electrical stimulus was inadvertently applied two millimeters below the optimal site of stimulation for Parkinson's symptom relief) (Bejjani et al., 1999; Hardesty and Sackeim, 2007). However, it also cautions that the therapeutic efficacy of any treatment heavily depends on the specificity of its delivery, as a small targeting error can induce potentially dangerous nonlinear side effects. The case study shows that DBS therapeutic efficacy is largely dose-dependent in addition to site-dependent (Fontaine et al., 2004; Hardesty and Sackeim, 2007). To date DBS has been clinically trialed and thus far failed to achieve their clinical endpoints. This may in part be due to the open loop nature of the treatment (failed to treat depressive symptoms when they occurred) and due to individual variability in disease pathologic and optimal target site for treatment. Also, the more invasive nature of the therapy may limit the number of potential candidates for future therapy.

### Brain Stimulation Targets

Few neural stimulation targets have been evaluated for treatment efficacy in the refractory MDD population. In general, proposed stimulation targets are linked to limbic structures and come directly from hypotheses of neural dysfunction in depression, imaging studies, unexpected mood improvements observed in stimulation studies treating other disorders, and areas accessible with a given stimulation technology. TMS studies typically target the left and/or right dorsolateral prefrontal cortex (DLPFC) due to its accessibility with the large stimulation coils and the promising history of its antidepressive effects. Slow TMS (sTMS) has only provided antidepressive effects when used on the right DLPFC (Klein et al., 1999; Fitzgerald et al., 2006), while repetitive/rapid TMS only has provided antidepressive effects when used on the left DLPFC (Speer et al., 2000; Avery et al., 2006; Fitzgerald et al., 2006). Not surprisingly, DBS studies target deep brain structures such as the subcallosal cingulate gyrus (SCG) (Mayberg et al., 2000, 2005; Lozano et al., 2008), ventral capsule/ventral striatum (VC/VS) (Malone et al., 2009), globus pallidus internus (GPi) (Kosel et al., 2007), and inferior thalamic peduncle (ITP) (Jimenez et al., 2005).

Each stimulation technology uses different sets of stimulation parameters, using a constant-current or voltage-based monophasic or biphasic waveforms with a diverse range of amplitudes, pulse durations, and stimulation frequencies (see Albert et al., 2009 for a comprehensive review of stimulation parameters that have been used for VNS, TMS, and DBS). The respective waveforms stimulate a target structure continuously or intermittently (in open-loop configurations) in hopes of directly or indirectly modulating abnormal activity toward more normal behavior in limbic-associated neural pathways and structures (e.g., VNS technology intermittently stimulates for 30 seconds every 5 minutes to indirectly modulate brain activity via the left cervical vagus nerve, (Marangell et al., 2007). DBS stimulation parameters are wirelessly programmed approximately 2 weeks after implantation on a patient-specific basis. By using patient-reported symptom relief and side effects, stimulation pulse duration and amplitude are steadily increased over a period of weeks to months (under a constant pulse repetition frequency) to determine a range of parameters that produce the most significant therapeutic benefit with the least side effects (monophasic, constant-current stimulation is typically used in VNS and monophasic, constant-voltage stimulation is typically used in DBS) (Hardesty and Sackeim, 2007). TMS devices first measure a patient's motor threshold (i.e., the magnetic pulse intensity that elicits a motor action potential when applied over the motor cortex) before beginning the procedure (Marangell et al., 2007). A percentage of the observed motor threshold is then used as the baseline intensity at which the magnetic pulse is applied for therapy (Albert et al., 2009).

Stimulation programming procedures are often uncomfortable for the patient, as severe side effects are often induced due to unintended neural stimulation from poorly placed stimulus transducers, poorly chosen parameters, and/or limited spatial resolution from a given stimulation technology. Increasing the specificity of stimulus delivery to more precisely target the dysfunctional neurons or networks should lead to reduced side effect profiles. Further timing the stimulation to match the clinical need for the patient's fluctuations will also enhance over all efficacy.

### BRIEF DESCRIPTION OF THE SEVERAL VIEWS OF THE DRAWINGS

Some embodiments of the invention are herein described, by way of example only, with reference to the accompanying drawings, in which like components are designated by like reference numerals. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic block diagram illustrating the components of a system for treating mood disorders, in accordance with some embodiments of the systems of the present application;

FIG. 2 is a schematic isometric view illustrating an intra-calvarial implant, usable in some embodiments of the systems for treating mood disorders of the present application;

FIG. 3 is a schematic bottom view of the intra-calvarial implant of FIG. 2;

FIG. 4 is a schematic side view of the intra-calvarial implant of FIG. 2;

FIG. 5 is a schematic cross-sectional view of the intra-calvarial implant of FIG. 2 taken along the lines V-V, also illustrating the position of the implant relative to the calvarial bone after implantation in the skull of a patient;

FIG. 6 is a schematic flow chart diagram illustrating the steps of a method for delivering brain stimulation therapy by processing sensed cortical activity and ecological momentary mood assessment data of a patient, in accordance with some embodiments of the methods of the present application;

FIG. 7 is a schematic flow chart diagram illustrating the steps of a method for assessing the correlation between one or more parameters of recorded cortical signals and a Mood index computed from ecological momentary mood assessment (EMA) data of a patient, in accordance with some embodiments of the methods of the present application.

FIGS. 8A-8B are schematic flow chart diagrams illustrating the steps of a method for delivering graded brain stimulation therapy to a patient by processing sensed cortical activity and ecological momentary mood assessment data of the patient, in accordance with some embodiments of the methods of the present application;

FIGS. 9A-9B are schematic flow chart diagrams illustrating the steps of a method for delivering brain stimulation therapy to a patient by using the value of the power at the gamma frequency band (PT) of sensed cortical signals and the ecological momentary mood assessment (EMA) data of the patient, in accordance with some embodiments of the methods of the present application;

FIG. 10 is a schematic flow chart diagram of a method for delivering graded stimulation therapy to a patient responsive to processing cortical signals, EMA data and additional sensor data, in accordance with some embodiments of the methods of the present application;

FIG. 11 is a schematic flow chart diagram of a method for delivering intermittent brain stimulation therapy to a patient responsive to processing cortical signals, EMA data and additional sensor data, in accordance with some embodiments of the methods of the present application;

FIG. 12 is a schematic block diagram illustrating a system for treating mood disorders including scalp electrodes for performing transcranial frequency interference stimulation of cortical and/or deep brain structures and intra-cranially implanted ECOG electrode arrays for sensing and/or stimulating one or more cortical regions, in accordance with some embodiments of the systems of the present application;

FIG. 13 is a schematic block diagram illustrating the functional components of an intra-cranial part of the system of FIG. 12;

FIG. 14. is a schematic drawing illustrating a system for treating a mood disorder having multiple intra-cranial ECOG arrays for performing sensing in one or more cortical regions and for performing trans-cranial frequency interference stimulation (TFIS) of one or more deep brain structures and/or direct stimulation of one or more cortical region(s), in accordance with some embodiments of the systems of the present application;

FIG. 15 is a schematic functional block diagram illustrating functional components included in the system of FIG. 14;

FIG. 16 is a schematic isometric view diagram illustrating a human skull with an implanted intra-calvarial implant suitable for delivering deeper brain stimulation to a patient's brain implanted in the calvarial bone of the skull in accordance with some embodiments of the intra-calvarial implants of the present application; and

FIG. 17 is a top view of the skull illustrated in FIG. 16.

### SUMMARY OF THE INVENTION

There is therefore provided, in accordance with some embodiments of the systems of the present application, a system for treating a mood disorder in a patient. The system includes one or more implantable devices, each device including one or more electrodes for sensing cortical signals in one or more cortical regions and for stimulating one or more regions of the brain. The system also includes one or more processor/controllers in communication with the one or more electrodes for receiving and processing sensed cortical signals and for controlling the stimulating of one or more brain regions through the one or more electrodes. The system also includes at least one portable communication device operable by the patient and having an application software operating thereon for acquiring ecological mood assessment (EMA) data representative of the momentary mood of the patient and for communicating the data to the at least one processor/controller(s) and/or to at least one remote processor. The data is processed by the one or more processor/controllers, and/or by a processor included in the portable communication device and/or by the at least one remote processor for modulating and/or controlling the stimulating of one or more brain regions to treat the mood disorder. The system also includes at least one power source suitably electrically connected to the one or more implantable devices for providing power thereto.

In some embodiments, the one or more implantable devices are selected from, one or more intra-cranially implantable devices, one or more implantable intra-calvarial devices and any combinations thereof.

In some embodiments, the one or more electrodes are selected from, one or more intra-calvarial electrodes, one or more intra-calvarial electrode arrays, one or more intra-cranial electrodes, one or more intra-cranial electrode arrays and any combinations thereof.

In some embodiments, at least one of the one or more implantable device(s) is an intra-calvarial device having intra-calvarial electrodes, disposed between an outer table and an inner table of the calvarial bone of the patient without fully penetrating the inner table of the calvarial bone.

In some embodiments, at least some of the electrodes of the intra-calvarial implant are in contact with an outer surface of the inner table of the calvarial bone.

In some embodiments the system includes one or more implantable frequency interference (FI) devices configured for stimulating one or more brain regions by using a frequency Interference stimulation method.

In some embodiments, the one or more brain regions stimulatable by the implantable FI devices are selected from, at least one cortical region, at least one deep brain structure and any combinations thereof.

In some embodiments the at least one cortical region being stimulated is selected from, the right dorsolateral prefrontal cortex (RDLPFC), the left dorsolateral prefrontal cortex (LSLPFC), one or more regions of the cingulate cortex, one or more regions of the prefrontal cortex (PFC) and any combinations thereof.

In some embodiments the at least one deep brain structure being stimulated is selected from, ventral striatum (VS), one or more parts of the limbic system, a subgenual cingulate region (BA 25), a ventral capsule (VC), a nucleus accumbens, a lateral habenula, a ventral caudate nucleus, an inferior thalamic peduncle, an insula, and any combinations thereof.

In some embodiments, the one or more cortical regions are selected from the right dorsolateral prefrontal cortex (RDLPFC), the left dorsolateral prefrontal cortex (LDLPFC), a region of the prefrontal cortex (PFC), and any combinations thereof.

In some embodiments, the system also includes one or more sensor units for sensing one or more additional biomarkers indicative of the patient's mood.

In some embodiments, the one or more sensor units are selected from, a heart rate sensor, a perspiration sensor, a pupilometry sensor, an AR headset 11, an eye tracking sensor, a microphone, a blood serotonin sensor, a blood dopamine sensor, and any combination thereof.

In some embodiments, the one or more biomarkers are selected from, a heart rate, a heart rate variability, blood pressure, a change in perspiration rate, a pupil size change in response to presentation of a negative word, an eye movement parameter, a change in vowel space of a patient's speech, a change in blood serotonin levels, a change in blood dopamine levels, and any combination thereof.

In some embodiments, the mood disorder is selected from, major depressive disorder (MDD), post-traumatic stress disorder (PTSD), anxiety, and any combinations thereof.

In some embodiments, the system also includes one or more effector devices controllable by the one or more processor/controller(s) and/or by the one or more communication device, the one or more effector device(s) are selected from, a device for delivering serotonin to the patient's brain, a device for delivering dopamine to the patient's brain and any combinations thereof.

In some embodiments, the one or more processor/controller(s) are programmed to process the cortical signals and the EMA data to determine the value of a mood index MX and to deliver stimulation to the one or more brain regions if the value of MX is smaller than or equal to a threshold level.

In some embodiments, the value of MX is computed from the cortical signals and of the EMA data or from the cortical signals, the EMA data and one or more patient's biomarker data sensed by one or more sensors.

In some embodiments, the one or more processor/controllers are programmed to process the cortical signals and the EMA data to determine the value of a mood index MX and to deliver graded stimulation to the one or more brain regions responsive to the value of MX.

In some embodiments, the mood index MX comprises a modulation index MI computed from the cortical signals and the EMA data.

There is also provided, in accordance with some embodiments of the systems of the present application, a system for treating a mood disorder in a patient. The system includes one or more intra-calvarial implants, each implant including a power source, a plurality of intra-calvarial electrodes for sensing cortical signals and for stimulating one or more regions of the brain, a telemetry module for communicating sensed cortical signals and/or data, and for wirelessly receiving data and/or control signals. At least some of the intra-calvarial electrodes are disposed between an outer table and an inner table of the calvarial bone of the patient without fully penetrating the inner table of the calvarial bone. Each of the one or more implantable intra-calvarial implants includes one or more processor/controllers in communication with the plurality of intra-calvarial electrodes for processing sensed cortical signals and for controlling the stimulating of the one or more regions of the brain. The system also includes at least one portable communication device operable by the patient and having an application software operating thereon for acquiring ecological mood assessment (EMA) data representative of the momentary mood of the patient and for communicating the EMA data to the one or more processor/controllers of the one or more implantable intra-calvarial implants and/or to at least one remote processor. The data is processed by the one or more processor/controllers of the one or more intra-calvarial implants and/or by a processor included in the portable communication device and/or by the at least one remote processor for modulating and/or controlling the stimulating of the one or more regions of the brain to treat the mood disorder.

In some embodiments of the systems of the present application, at least one portable communication device is selected from, a mobile phone, a smartphone, a laptop, a mobile computer, a tablet, a notebook, a phablet, an augmented reality (AR) headset and any combinations thereof.

There is also provided in accordance with some embodiments of the methods of the present application, a method for treating a mood disorder of a patient. The method include the steps of receiving cortical signals sensed from one or more cortical regions of the patient, automatically receiving ecological mood assessment (EMA) data of the patient from at least one portable communication device operated by the patient, the at least one communication device has an application software operative thereon for automatically obtaining data representing the parameters of use of the at least one portable communication device by the patient to locally compute the EMA data and/or to receive computed EMA data from a remote processor, processing the cortical signals and the EMA data to detect an indication that the patient is in a depressed mood requiring therapeutic stimulation, and stimulating at least one brain region of the patient responsive to detecting the indication.

In accordance with some embodiments of the method, the signals of the step of receiving are recorded by one or more implants selected from, extra-cranial implants, intracranial implants, intra-calvarial implants, and any combinations thereof.

In accordance with some embodiments of the method, the signals of the step of receiving are recorded by one or more intra-calvarial electrodes. At least some of the intra-calvarial electrodes are disposed between an outer table and an inner table of a calvarial bone of the patient without fully penetrating the inner table of the calvarial bone.

In accordance with some embodiments of the method, the one or more intra-calvarial electrodes are disposed in contact with or adjacent to an outer surface of the inner table of the calvarial bone.

In accordance with some embodiments of the method, the EMA data includes data selected from, automatically obtained data representing multiple parameters of use of the at least one portable communication device by the patient, and data representing a subjective mood assessment provided by the patient in response to a request for a mood assessment automatically presented to the patient.

In accordance with some embodiments of the method, the EMA data includes data selected from, data representing application use by the patient, data representing number of calls made by the patient, acceleration data due to patient's movements, communication data, ambient light data, ambient sound data, patient's location data, patient's call log, patient's voice content, patient's texting content, patient sleep data, patient's social network data, and any combinations thereof.

In accordance with some embodiments of the method, the step of automatically receiving also includes the step of automatically receiving biomarker data from one or more sensors, and wherein the step of processing comprises processing the cortical signals, the EMA data and the biomarker data to detect an indication that the patient is in a depressed mood requiring therapeutic stimulation.

In accordance with some embodiments of the method, the step of processing includes processing sensed cortical signals and the EMA data to compute a value of a modulation index parameter MI and/or to compute a patient's mood index MX.

In accordance with some embodiments of the method, the step of processing includes processing the sensed cortical signals and the EMA data and biomarker data obtained from one or more sensors to compute a value of a modulation index parameter MI and/or to compute a patient's mood index MX.

In accordance with some embodiments of the method, the step of processing comprises processing the sensed cortical signals by computing the spectral power in one or more spectral bands, computing a modulation index MI and/or computing a mood index MX.

In accordance with some embodiments of the method, the step of processing includes a comparing the value of MI to a threshold value, and the step of stimulating comprises stimulating one or more brain regions if the value of MI is equal to or larger than the threshold value.

In accordance with some embodiments of the method, the step of processing includes comparing the value of a mood index MX to a threshold value, and the step of stimulating comprises stimulating one or more brain regions if the value of MX is equal to or larger than the threshold value.

In accordance with some embodiments of the method, the step of stimulating includes stimulating one or more brain regions, selected from one or more cortical brain regions, one or more deep brain structure and any combinations thereof.

In accordance with some embodiments of the method, the one or more cortical brain regions of the step of stimulating are selected from a right DLPFC, a left DLPFC, a region of the PFC, a subgenual cingulated cortex, and any combinations thereof, and the one or more deep brain structures of the step of stimulating are selected from a ventral striatum (VS), one or more parts of the limbic system, a subgenual cingulate region (BA 25), a ventral capsule (VC), a nucleus accumbens, a lateral habenula, a ventral caudate nucleus, an inferior thalamic peduncle, an insula, and any combinations thereof.

In accordance with some embodiments of the method, the step of receiving comprises receiving cortical signals from one or more cortical regions selected from a right DLPFC, a left DLPFC, a region of the PFC and any combinations thereof.

In accordance with some embodiments of the method, the mood disorder is selected from, major depressive disorder (MDD), post-traumatic stress disorder (PTSD), anxiety, and any combinations thereof.

There is also provided in accordance with some embodiments of the methods of the present invention, a method for treating a mood disorder of a patient. The method includes the steps of receiving electrical signals recorded from a cortical region of the patient using an intra-calvarial implant comprising one or more intra-calvarial electrodes, at least one part of the intra-calvarial electrodes is disposed between an outer table and an inner table of the calvarial bone of the patient without fully penetrating the inner table of the calvarial bone, processing the signals to determine a stimulation paradigm for the patient, and stimulating at least on brain region of the patient responsive to the determined stimulation paradigm.

In some embodiments of the method, the method also includes the step of automatically receiving momentary mood assessment data for the patient from at least one portable communication device operated by the patient, the at least one communication device has an application software operative thereon for automatically processing data representing the parameters of use of the at least one communication device by the patient without patient intervention and to compute a momentary mood assessment and the step of processing includes processing the momentary mood assessment and the electrical signals to determine a stimulation paradigm for the patient.

In some embodiments of the method, the method also includes the step of interacting with the patient through the at least one portable communication device to receive voluntary patient input representing the patient's subjective mood assessment, and wherein the step of processing includes processing the patient's subjective mood assessment and the electrical signals to determine and/or modify a stimulation paradigm for the patient.

In some embodiments of the method, the method also includes the step of interacting with the patient through the at least one portable communication device to receive voluntary patient input representing the patient's subjective mood assessment, and wherein the step of processing includes processing the patient's subjective mood assessment, the EMA data and the electrical signals to determine and/or modify a stimulation paradigm for the patient.

In some embodiments of the method, the method also includes the step of receiving from at least one portable communication device ecological mood assessment (EMA) data representative of the momentary mood of the patient, and wherein the step of processing includes processing the signals and the EMA data to determine a stimulation paradigm for the patient.

In some embodiments of the method, the step of receiving also includes receiving from the patient voluntary mood assessment data in response to a system enquiry, and wherein the step of processing includes processing the signals and the EMA data and patient's voluntary mood assessment data to determine a stimulation paradigm for the patient.

Finally, in some embodiments of the methods of the present application, the at least one portable communication device is selected from, a mobile phone, a smartphone, a laptop, a mobile computer, a tablet, a notebook, a phablet, an augmented reality (AR) headset and any combinations thereof.

### DETAILED DESCRIPTION OF SPECIFIC EMBODIMENTS OF THE INVENTION

The systems and methods disclosed in the present application disclose a multiple closed loop cortical neuromodulation system that delivers brain electrical stimulation therapy based on sensed patient's cortical signals and on one or more relevant patient inputs in the form of ecological momentary assessments and/or other patient's physiological biomarkers. The “Patient and Sensor Informed Closed-loop Cortical” (PASICC) neuromodulation system does not require a priori identification of cortical signal or a physiological biomarker, but rather learns the biomarker with ongoing utilization by the patient. The system may include an intra-calvarial implant that is capable of stimulating and recording from a focal region in the cortex, a mobile communication device (such as, for example, a mobile phone, a smartphone a laptop, a tablet, a notebook, a phablet, an augmented reality (AR) headset having communication capabilities) that can engage with the patient to either actively or passively provide patient's mood assessments such, for example, ecological momentary mood assessment (EMA) to the system. The system also includes software for correlating the sensed cortical electrical activity with mood assessments to enable the detection of a mood state requiring treatment and deliver a selected stimulation regime. The system may adapt to each individual patient by using suitable training and/or test periods and may provide patient specific cortical biomarkers that may be used for optimized cortical stimulation to address the mood related symptoms of a patient with depression.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways. It is expected that during the life of a patent maturing from this application many relevant types of electrodes and electrode arrays will be developed and the scope of the terms “electrode” and “electrode array” are intended to include all such new technologies a priori. As used herein the term “about” refers to ±10%. The word “exemplary” is used herein to mean “serving as an example, instance or illustration.” Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments.” Any particular embodiment of the invention may include a plurality of “optional” features unless such features conflict.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

### Patient and Sensor Informed Closed-Loop Cortical (PASICC) Neuromodulation System for Depression

The PASICC neuromodulation system overcomes a number of the existing barriers to create a personalized treatment for depression. The system may include 1) an intra-calvarial implant (or, in some embodiments, other types of cranial or intra-cranial implants) that is capable of stimulating and recording from a focal region in the cortex, 2) a mobile communication device, such as, for example, a mobile computer or another portable (and/or wearable communication device (e.g. cellular phone or a smartphone or an AR headset having communication capabilities) that may engage with the patient to either actively or passively (and unobtrusively) provide mood assessments, such as, for example, ecological momentary assessments (EMA), 3) One or more software programs or applications for integrating and connecting cortical physiology with mood assessments to inform stimulation regime.

By creating a multiple closed loop cortical neuromodulation device that both incorporates cortical signals for neuromodulation and relevant patient input in the form of ecological momentary assessments the system can derive patient specific biomarkers that will define the optimal stimulation regime to aid in the patients improved mood. The system does not require a priori identification of cortical signal or a physiological biomarker, but rather learns the biomarker with ongoing utilization by the patient. As the system operates, it may “learn” patient specific cortical biomarkers that can inform optimal cortical stimulation to address the mood related symptoms of a patient with depression.

The system may operate in the following manner. The intra-calvarial implant may be implanted in the skull of the patient overlying cortical sites that would be useful to be stimulated for treating depression. The location of implantation may be defined by both anatomic and functional imaging. In accordance with some embodiments of the system, the dorsal lateral prefrontal cortex (DLPFC) may be chosen anatomically. More specific regions could be chosen using functional MRI. There are numerous types of functional MRI that could aid in localization. Specifically this may include resting state functional MRI to identify critical networks (e.g. dorsal attention network and default mode network), task based fMRI to elicit cortical activation in relevant regions, and diffusion tensor imaging (DTI) to identify critical white matter tracts adjacent to areas of stimulation. The intra-calvarial implant may be wirelessly connected to the user's mobile phone. The mobile phone or other communication device would include a software application and may have computational capabilities or access to such computational capabilities (either on by using the processor on the phone or by communicating with a computer having the required processing power (for example a cloud server wirelessly accessible by the phone over the internet) to process the data recorded from the patient's brain and the stimulation parameters and/or mood associated data provided by the patient and/or measured by sensors attached to the patient or found on the mobile phone.

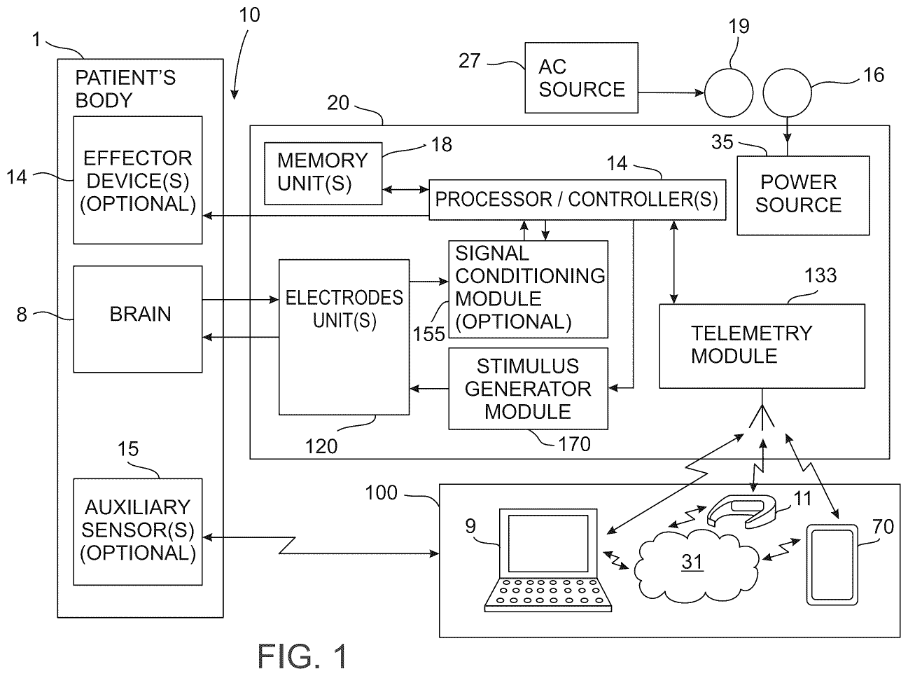

Reference is now made to FIG. 1 which is a schematic block diagram illustrating the components of a system for treating mood disorders, in accordance with some embodiments of the systems of the present application.

The system 10 may include an intra-calvarial implant 20, one or more communication devices 100 and (optional) auxiliary sensor(s) 15 implanted in or attached to or worn on the patient's body 1. The system 10 may also (optionally) include one or more effector device(s) 13. The effector device(s) 13 may be connected to the processor/controller(s) 14 to receive therefrom control signals for controlling the operation thereof. For example, the effector device(s) 13 may include one or more therapeutic devices (such as, for example a neurotransmitter or neuromodulator delivery device, capable of delivering a neurotransmitter and/or a neuromodulator to the patient's brain, such as the serotonin delivery device and/or the dopamine delivery device disclosed in more detail hereinafter).

The communication unit(s) 100 may include one or more devices having communication capabilities and may also have some processing capabilities. For example, the communication unit(s) 100 of FIG. 1 may include a mobile phone 70 and/or a laptop 9 and an AR headset 11. Other options for communication units may include tablets and/or phablets and/or notebooks that may have a communication capability enabling them to wirelessly communicate with the telemetry module 133 of the implant 20, and/or with each other, and/or with a server on the cloud.

The implant 20 may include one or more processor/controller units 14, suitably connected to memory unit(s) 18. The memory unit(s) 18 may be any suitable type of memory known in the art. Non limiting, exemplary memory and/or data storage devices usable in the system 10 may include one or more devices such as read only memory (ROM), random access memory (RAM), electrically programmable read only memory (EPROM), erasable electrically programmable read only memory (EEPROM), Flash memory devices, optical memory, and/or storage devices or any other type of memory known in the art, and any combinations thereof. It is noted that the memory unit(s) 18 may also be memory unit(s) integrated into the processor/controller(s) 14.

The processor/controller(s) 14 may be any type of processor(s) or controller(s) known in the art, such as, for example, a CPU, a microprocessor, a microcontroller, a digital signal processor (DSP) a graphic processing unit (GPU), an optical processor, a quantum computing device, and any combinations thereof.

The implant 20 may also include electrode unit(s) 120. The electrode unit(s) 120 may be any suitable type of electrodes for sensing electrical activity in one or more regions of the brain 8 of the patient and for stimulating one or more regions of the patient's brain 8. Some or all of the electrodes of the electrode unit(s) may be suitably coupled to a stimulus generator unit 170 included in the implant 20 for delivering electrical stimuli to the electrodes included in the electrode unit(s) for stimulating one or more regions of the brain 8. The stimulus generator unit 170 is suitably connected to the processor/controller(s) 14 for receiving control signals therefrom. The processor/controller(s) 14 may control the operation of the stimulus generator module 170. Some or all of the electrodes in the electrode unit(s) 120 may be suitably electrically connected to an (optional) signal conditioning module 155 that may be suitably connected to the processor/controller(s) 14. The signal conditioning module 155 may include all electronic/electrical circuits that may be necessary for filtering and/or amplifying and/or multiplexing and or digitizing the signals sensed by the electrodes unit(s) in a region of the brain 8 (such as, for example, filter circuits, band limiting circuits, multiplexing circuits, and analog to digital converting circuits, clocks or any other necessary electronic circuits). Alternatively, such circuits or some of them may be included in the processor/controller(s) 14.

The implant 20 may also include a telemetry module 133 suitably connected to the processor/controller(s) 14. The telemetry module may be any suitable module capable of wirelessly communicating data and/or control or command signals to the communication unit(s) 100 and to receive from the communication unit(s) 100 data and/or control signals. The telemetry module 133 may use any suitable type of communication protocol and frequency band to communicate with the communication unit(s) 100. For example, the telemetry module may use a RF signals and a cellular communication protocol to communicate with the mobile phone 70. Alternatively or additionally the telemetry module 133 may use WiFi protocol and/or a Bluetooth protocol to communicate with the mobile phone 70 and/or with the laptop 9 and/or with the AR headset 11.

Preferably, the laptop 9 (if a laptop is included in the system 10) may be connected wirelessly (or in a wired way) to the cloud 31 via WiFi and the internet. The mobile phone 70 may, preferably, also be wirelessly connected to the cloud 31(through WiFi and/or cellular data networking) and the AR headset 11 may be wirelessly connected to the mobile phone 70 and/or to the laptop 9 and/or to the cloud 31 using any suitable communication protocols and methods. Such wireless communication means may enable the processor/controller(s) 14 to wirelessly communicate with external devices, such as for example, a remote computer, a server (on the cloud 31), a cellular telephone (such as, for example, the mobile phone 70), an AR headset (such as, for example, the AR headset 11) or any other type of computer reachable through the cloud 31. This may be useful in cases in which the processing power of the processor/controller(s) 14 of the implant 20 is limited, as this may allow the offloading of some or all of the computational burden from the processor/controller to other processing devices, such as remote computer(s), remote servers, a cluster of computers or any other suitable computing devices, and may enable the use of cloud computing, or parallel computing for processing the data recorded/sensed reducing the computational load on the processor/controller(s) 14. The results of such off loaded computations may then be returned or communicated (preferably wirelessly) to the processor/controller(s) 14 and used for performing the controlling of the sensing and/or stimulation of the appropriate brain structures as disclosed hereinafter.

The implant 20 may also include a power source 35 for providing power to components of the implant 20. The power source 35 may be any suitable type of power source, such as, for example a suitable electrochemical cell, a rechargeable electrochemical cell, a fuel cell, a super capacitor or any other type of suitable power source. However, preferably, the power source 3 may be a power harvester. For example, the specific example of the power source 35 illustrated in FIG. 1 is implemented as a power harvesting device having an implantable induction coil 16 that may be implanted together with the implant 20 in the body 8 of the patient. The induction coil 16 may be energized by an external induction coil 19 that is connected to an external alternating current (AC) source 27. In this particular, the part of the power source 35 included within the implant 20 may also include suitable electronic/electrical circuitry (not shown in detail for the sake of clarity of illustration) for rectifying the AC induced in the induction coil 16 into direct current (DC) and a charge storing unit (not shown in detail), such as, for example a suitable super-capacitor and/or a rechargeable electrochemical cell.

It is noted that for the sake of clarity of illustration, the leads or wires connecting the power source 35 to other components of the implant 20, are not shown in detail.

The auxiliary sensor(s) 15 of the system 10 may be one or more sensors for sensing one or more properties of the patient's body 1. For example, the auxiliary sensor(s) 15 may include one or more of the following sensors, a temperature sensor, a perspiration sensor, a heart rate sensor, an eye tracking sensor, a pupil size sensor blood pressure sensor, an accelerometer, a chemical sensor, or any other type of sensor known in the art. The sensors may be implanted in the patient's body 1 and/or attached to the patient's body 1, and/or worn by the patient or attached to a garment worn by the patient. Alternatively or additionally, some of the sensors may be included in or integrated within one of the communication unit(s) 100. For example, modern smartphone may include heart rate metering applications as well as pupil size metering applications which may be easily used for determining the heart rate and pupil size of the patient.

In accordance with some embodiments of the systems, some of the sensors may be included in the AR headset (such as, for example in the AR headset 11), and may include, eye tracking sensors, pupil size sensors, accelerometers, movement sensors, microphones, perspiration sensors, heart rate sensors, or any other type of suitable sensors that may be integrated into an AR headset. This may have the advantage of making the system more compact. In some of the embodiments of the system, the AR headset may integrate all the functions and capabilities of the mobile phone 70, as well as the computations functions of the laptop 9 making the mobile phone 70 and the laptop 9 redundant.

As AR headsets are gradually becoming less cumbersome more lightweight and more computationally powerful, some embodiment of the systems disclosed in the present application may include, one AR headset 11, one or more implants (such as the implant 200 or the implant 180 described in detail hereinafter). The AR headset 11 may be able to communicate with the cloud 31 and may be used to offload data from the implant(s), communicated data (including EMA data, sensor data and all other types of data) to and from a remote computer/server on the cloud 31 and may also process some of the data and send command signals to the implants for controlling the stimulation and sensing of the implants. In such an embodiment, the power source may be a power source included in the AR headset 11 and powering the implant(s) through suitable power leads connected from the AR headset 11 to the implants.

If the sensors are not included in the mobile phone 70 or the laptop 9 (such as, for example the auxiliary sensors 15), the sensors may be sensors implanted in or attached to the body of the user or worn by the user, in which case such sensors may include wireless communication circuitry (not shown in detail) that may enable the sensors to wirelessly transmit to the signals and/or data sensed by the sensors to the telemetry module 133 and/or to the mobile phone 70 and/or to the laptop 9 for storage and/or processing. In this way the system 10 may sense one or more parameters including physical parameters (such as, for example, body acceleration or movements) and/or physiological parameters (such as, for example, body temperature, pupil size and/or variations thereof, perspiration rate, heart rate or other physiological parameters).

An example of a sensor worn by the patient is the model Tobii Pro 2 wearable eye-tracker commercially available from Tobii A B, Stockholm, Sweden. This eye-tracker is a lightweight spectacle-like unit that may be worn by the user and may provide the patient's eye tracking data and the patient's pupil size data.

It is noted that, in accordance with some embodiments of the system 10, one or more of the auxiliary sensor(s) 15 may be implanted chemical sensors for determining the blood concentration of neurotransmitters (such as, for example, a serotonin sensor and/or a dopamine sensor). Such sensors may provide the processor/controller(s) 14 and/or the mobile phone 70 and/or the laptop 9 with data representing the concentration of serotonin and/or dopamine in the patient's blood. This data may also be processed by the system 10 and may be used in the calculation of the value of the mood index (MX) disclosed hereinafter with respect to the methods.

Such neurotransmitter concentration data may also be used to automatically control the operation of one or more devices of the effector device(s) 13 of FIG. 1. For example, one or more of the effector device(s) 13 may be a neurotransmitter delivering device capable of delivering serotonin and/or dopamine to the relevant region(s) of the patient's brain on demand. Such neurotransmitter delivery device(s) or only their parts for neurotransmitter delivery (such as, for example a suitable cannula) may be implanted in the patient's skull. If the blood transmitter level drops below a preset or predetermined threshold, the processor/controller(s) 14 may activate the neurotransmitter delivery device(s) to deliver a therapeutic dose of serotonin and/or dopamine to the patient's brain or to the patient's blood (this chemical therapy may be performed independently of the therapeutic brain stimulation or together with the therapeutic brain stimulation).

Some methods of operation of systems such as the system 10 are disclosed in more detail hereinafter.

The implant 20 may be implemented in various different embodiments. In accordance with some embodiments of the system, the implant 20 may be an intra-calvarial implant.

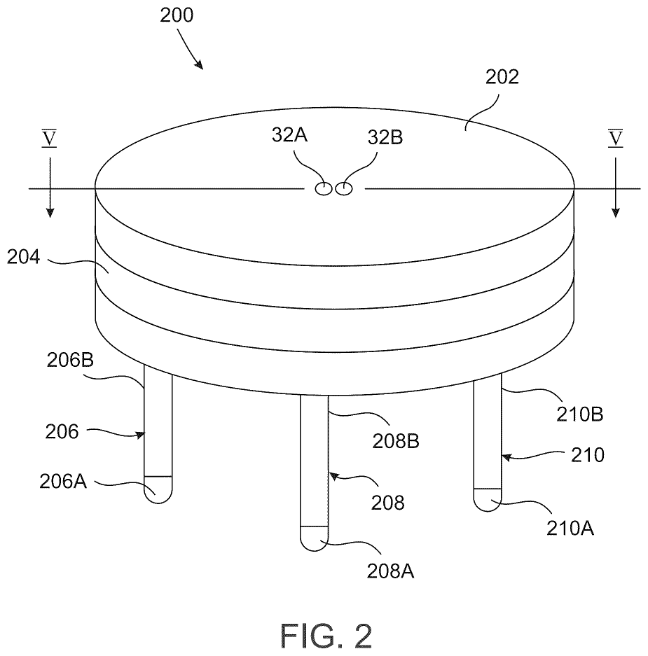

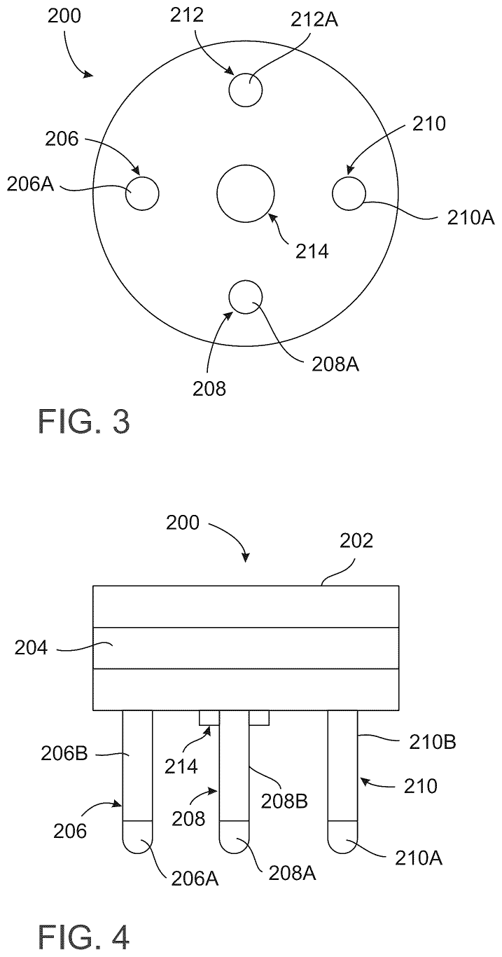

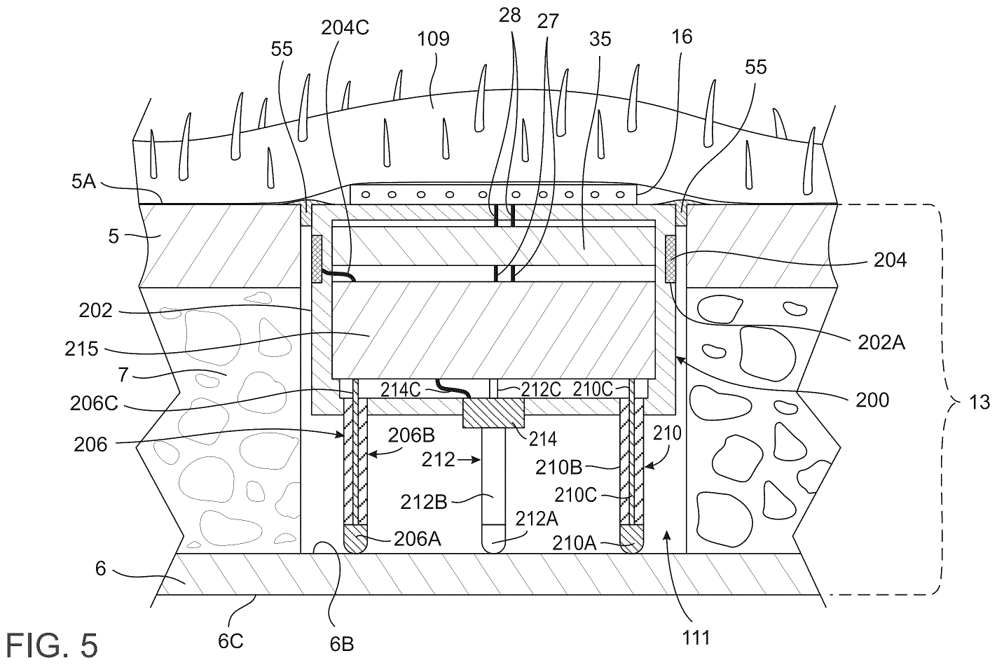

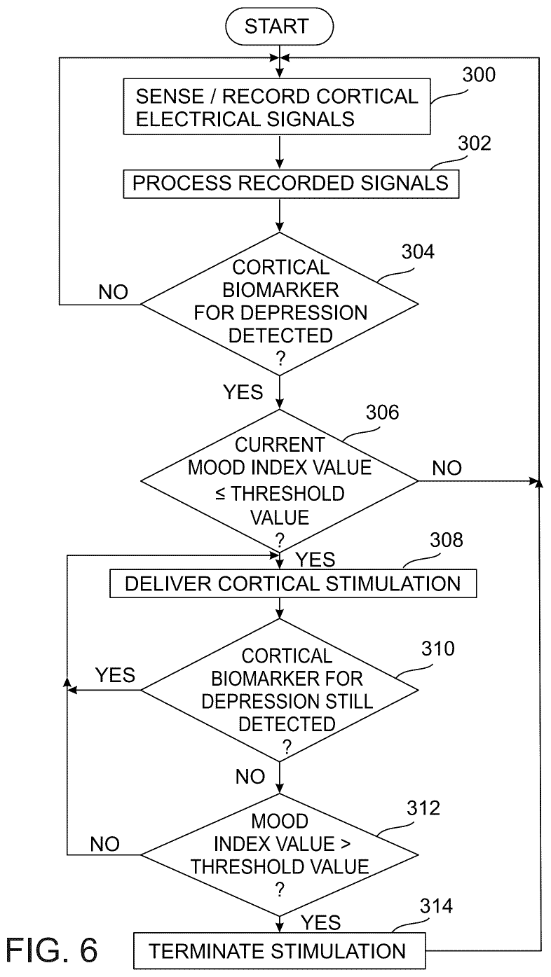

Reference is now made to FIGS. 2-5. FIG. 2 is a schematic isometric view illustrating an intra-calvarial implant, usable in some embodiments of the systems for treating mood disorders of the present application. FIG. 3 is a schematic bottom view of the intra-calvarial implant of FIG. 2. FIG. 4 is a schematic side view of the intra-calvarial implant of FIG. 2. FIG. 5 is a schematic cross-sectional view of the intra-calvarial implant of FIG. 2, taken along the lines V-V, also illustrating the position of the implant relative to the calvarial bone after implantation in the skull of a patient.

The intra-calvarial implant 200 may include a housing 202. The housing 202 may be a cylindrical or disc-like housing, but other housing shapes may also be used. The housing 202 may be made from any suitable biocompatible material, such as, for example titanium, stainless steel, a polymer based material, Parylene® or any other suitably strong biocompatible structural material. The intra-calvarial implant 200 also includes four electrodes 206, 208, 210 and 212, a reference electrode 214 and a ground strip 204. If the housing is made from an electrically conducting metal, the ground strip 204 may be electrically isolated from the housing by a layer of non-electrically conducting material (not shown) disposed between the housing 202 and the ground strip 204. If the housing 202 is made from a non-electrically conducting material, the ground strip 204 may be a thin layer of conducting material (such as, gold or platinum) coating the outside facing surface of the housing 202, alternatively (as illustrated in FIG. 5), the ground strip 204 may be disposed in a recess 202A formed in the side walls of the housing 202.

Turning to FIGS. 3-4, each of the electrodes 206, 208, 210 and 212 has an electrode tip 206A, 208A, 210A and 212A, respectively and an electrode shank 206B, 208B, 210B and 212B, respectively. The electrode tips 206A, 208A, 210A and 212A, the reference electrode 214 and the ground strip 204 may be made from an electrically conducting material (such as, for example, gold, platinum, stainless steel, stainless steel coated with gold or platinum or from any other biocompatible electrically conducting material). The electrode shanks 206B, 208B, 210B and 212B may be made from an electrically isolating material (such as for example, a non-electrically conducting polymer based material, Parylene®, or any other suitable biocompatible polymer. The reference electrode 204 may be made from the same electrically conducting material of the electrode tips 206A, 208A, 210A and 212A.

Turning to FIG. 5, the intra-calvarial implant 200 is illustrated as implanted in the calvarial bone of the patient's skull. The housing 202 of the implant 200 is implanted in a cavity 111 surgically made within the calvarial bone 13 (by drilling, burring or any other suitable surgical methods). The cavity 111 opens at the outer surface 5A of the outer table 5 of the calvarial bone 13 and extends through the cancellous bone layer 7, reaching the outer surface 6B of the inner table 6 of the calvarial bone 13.

It is noted that the shape and dimensions of the cavity 111 as illustrated in FIG. 5 are not obligatory. For example, in some embodiments, the cavity 111 may be shaped to accommodate the housing 202 and the reference electrode 214 and to include four narrow passages (not shown in FIG. 5) reaching the inner table 6. The electrodes 206, 208, 210 and 212 may be inserted into the fitting four narrow passages formed in the cancellous bone 7 such that the electrode tips 206A, 208A, 210A and 212A are in contact with or very near to the outer surface 6B of the inner table 6. The advantage of such a cavity configuration is that it minimizes the amount of cancellous bone that has to be drilled into and removed.

It is noted that in some embodiments, the cavity 111 may partially extend into the inner table 6 (not shown in the embodiment illustrated in FIG. 5) by carefully penetrating the surface 6B to extend the cavity 111 into the inner table 6 without breaching the inner table 6 (i.e. without fully penetrating the inner table 6). This may advantageously reduce the thickness of the boney material intervening between the electrode tips 206A, 208A, 210A and 212A which may result is reduced attenuation of the cortical signal recorded from the cortical region (not shown) underlying the inner table 6. Additionally, reducing the thickness of the inner table 6 may advantageously improve the stimulation of the cortex by the electrodes 206, 208, 210 and 212, by reducing the current intensity required for stimulation and thus, saving power.

The implant 200 may include the power source 35 (not shown in detail in the cross sectional view of FIG. 5) and an electronics module 215. The electronics module 215 may include the processor/controller(s) 14, the memory unit(s) 18, the signal conditioning module 155, the stimulus generator module 170 and the telemetry module 133.

The power source 35 may be any suitable type of power source, Such as, for example, a battery or electrochemical cell (primary cell or rechargeable cell), super-capacitor, fuel cell or any other suitable type of power source. Alternatively or additionally, the power source 35 may be a power harvesting device capable of receiving energy and storing the energy as stored charge. For example, one possible embodiment of the power source 35 is coupled to an induction coil 16 as disclosed in detail hereinafter and illustrated in FIG. 5.

Alternatively and/or additionally, the power source may include any type of suitable power harvesting device for receiving or producing power and storing the received or produced power. For example, the power source 35 may include a piezoelectric element for receiving acoustic energy from an external sound or ultrasound generator placed close to the implant 200. In another embodiment, the power source 35 may include an electro-mechanical generator device that converts patient's head or body movements into storable electrical charge. Such power harvesting devices are not the subject matter of the present invention, are well known in the art, and are therefore nor described in detail hereinafter.

It is noted that for implants that may require substantial amounts of power for operation it may be possible to replace the power source 35 that is disposed internally within the implant 200 (or within any other implant disclosed in the present application) with a power source (not shown) that is implanted in the patient's body or is carried or worn by the patient. In some embodiment, a medical surgically implantable power source (not shown) may be implanted in the patient's body and suitably electrically coupled to the implant (such as the implant 200) through suitable leads (not shown) that may enter the implant 200 through the hollow passages 32A and 32B as disclosed hereinafter (see FIG. 2). For this purpose any of the implantable power sources used to energize pacemakers and/or defibrillators may be used, as is known in the art of pacemakers and defibrillators. For example, such power sources may be implanted in a suitable subcutaneous pocket made in the patient's chest and connected to the implant by suitable leads. Any other suitable implantation methods and location of implantation for such medical power sources may also be used.

The electrode tips 206A, 210A and 212A may be connected to the electronics module 215 by suitable electrically conducting wires 206C, 210C and 212C (which may be, preferably, insulated electrically conducting wires). It is noted that the electrically conducting wire connecting the electrode tip 208A to the electronics module 215 is not shown in the cross-sectional view of FIG. 5. The reference electrode 214 may be electrically connected with the electronics module 215 by an insulated electrically conducting wire 214C. The ground strip 204 may be connected to the electronics module 215 by an insulated electrically conducting wire 204. The electronics module 215 may be connected to the power source 35 by a pair of suitable electrically conducting insulated wires 27.

The power source 35 may be electrically coupled to the induction coil 16 by a pair of electrically conducting insulated wires 28 sealingly passing through two suitable hollow passages 32A and 32B (see FIG. 2) formed within the housing 202. The induction coil 16 of FIG. 5 is illustrated as disposed between the housing 202 and the scalp 109 of the patient after implantation. The patient may periodically charge the power source 35 by placing the induction coil 19 (not shown in FIG. 5) on the scalp region overlying the induction coil 16 and passing alternating current from the AC source 27, through the induction coil 19.

### Stimulation Specifications

In accordance with some embodiments, each of the four electrodes 206, 208, 210 and 212 may be capable of independent and concurrent biphasic electrical sourcing. Typically, an asymmetric, charge-balanced bi-phasic waveform may be sourced/sinked concurrently from all four electrodes 206, 208, 210 and 212. The magnitude of the current (typically, up to 6 milliampere (mA)) in each of the four (source) electrodes 206, 208, 210 and 212 is independent of one another and programmable. If all four electrodes 206, 208, 210 and 212 are maximally active, the total current from the entire implant 200 may be 24 mA. The electrical return path for all four electrodes 206, 208, 210 and 212 may be the large ground strip 204 on the housing 202. Each independent electrode of the four electrodes 206, 208, 210 and 212 may have a compliance voltage of up to 12 volts. The reference electrode 214 is typically not used for stimulation. Standard cortical stimulation parameters may be telemetrically programmed into the implant 200. In some embodiments of the system 10, the stimulation parameters may be in the following ranges, pulse width in the range of 5-750 microsecond (μS) and pulse frequency in the range of frequency 5-500 Hertz (Hz). However, other values of the parameters outside (lower or higher than) the above ranges may also be used.

### Recording Specifications

The four (source) electrodes 206, 208, 210 and 212 may also be capable of recording voltage-based field potentials. In accordance with one embodiment of the implant 200, The implant 200 will not stimulate and record concurrently but rather may be quickly interlaced between recording and stimulation modes (For example, using an interleaved stimulating and recording periods having a duration smaller than 100 millisecond; or an alternation frequency greater than 10 Hz). Each electrode of the electrodes 206, 208, 210 and 212 may be differentially recorded relative to the slightly larger centrally placed reference electrode 214 which may be impedance-matched to the four (sourced) electrodes 206, 208, 210 and 212. The ground strip electrode 204 may be positioned in the vicinity of the outer table of the calvarial bone of the skull (see FIG. 5). The reference electrode 214 may be disposed in the cavity 111 within the central marrow of the cancellous bone layer 7 of the calvarial bone. The electrodes 206, 208, 210 and 212 may be positioned such that their electrode tips 206A, 208A, 210A and 212A are in the vicinity of or in contact with the outer surface 6B of the inner table 6 of the calvarial bone (as seen in FIG. 5).

The frequency range for recording may be e in the range of 3-200 Hz. The noise floor in the mid-gamma band (75-105 Hz) may be less than 200 nanovolt (nV). The maximum differential field potential is 100 microvolt (μV). However, the amplifier(s) (not shown in detail) included in the electronics module 215 may be capable of a single ended input (e.g. electrode 206 to ground strip 204) of up to a 5 millivolt (mV). After unity-gain differential recording with a maximum input of +/−5 mV, the signal may be band-passed filtered (3-200 Hz) and amplified with a gain of about 50×. A 12 bit analog to digital converter (A/D) with a maximum input of +/−5 mV may sample at a minimum rate of 2 kHz (10× sampling). With the 50× gain and a maximum input range of +/−5 mV, the A/D sampling voltage resolution may be less than 50 nV.

In operation of the system 10, with continued usage the patient would either be episodically “pinged” (questioned or queried) by the communication unit(s) 100 (such as, for example by using the mobile phone 70) to receive information or data about the patient's current emotional state (or mood). The information received may be used to correlate the mood state with given cortical physiological parameters. These parameters may include frequency band amplitude, frequency phase interactions, frequency band amplitude ratios, phase amplitude coupling at a given electrode and between different recording electrodes. Using a machine learning algorithm (e.g. support vector machines, deep learning, multi-level neural networks, etc.) a statistical model may be then created to predict the mood states from the physiological signals.

As the statistical model gradually emerges with use of the system 10, stimulation parameters may be constructed to stimulate the brain that induce the physiologic state that best predicts positive mood states. Basic stimulation parameters may be set at the outset, but would be subject to modification with ongoing closed loop interaction. In accordance with some system and method embodiments, such stimulation parameter modifications may occur automatically. Alternatively and/or additionally, modifications of stimulation parameters may be performed by a caretaker of the patient such as a psychiatrist or another medical caretaker monitoring the patient.

This multi-loop system may continually optimize with ongoing input from the patient. As the patient intermittently provides input to the application operating on the mobile phone 70, the system 10 continually operates to improve the accuracy of biomarker's indication of patient's positive or negative mood.

### Automatic and Voluntary EMA Assessment Methods

To collect self-monitored mood data (the target of the prediction task), the system 10 may use eMate, an EMA mobile phone application developed at the Vrije Universiteit Amsterdam. This application prompts participants to rate their mood on their smartphone at five set time points per day (i.e., approximately at 09:00, 12:00, 15:00, 18:00, and 21:00). As shown in the article by Robert LiKamWa et al (2013), cited in the reference list below, mood may be assessed through the circumplex model of affect [see article by Robert A Russel (1980) cited in the reference list hereinafter], which conceptualizes mood as a two-dimensional construct comprising different levels of valence (positive/negative affect) and arousal. Levels on both dimensions may be tapped on a 5-point scale scored from −2 to 2 (low to high). Because recent studies suggest that single-item mood measures can provide predictive information on the development of depressive symptoms (for details see Gerard D. van Rijsbergen et al 2012 article in the reference list hereinafter), one may also add a one-dimensional mood question, which asked participants to rate their current mood on a 10-point scale, with 1 as the negative and 10 as the positive pole.

### Unobtrusive Ecological Momentary Assessment of Mood Predictors

For unobtrusive assessment, the systems/methods of the present application may use iYouVU, a faceless mobile phone application based on the Funf open-sensing-framework (Aharony, N., Gardner, A., Sumter, C., & Pentland, A. (2011). Funf: Open sensing framework.), and prior research into communication habits based on mobile phone data collected without the user's full awareness. This application runs in the background, unnoticeable to the user, to collect designated sensor data and application logs. The application logs call events (i.e., time/date of the call, duration, and contact of both incoming and outgoing calls), short message service (SMS) text message events (i.e., time/date and contact), screen on/off events (i.e., time/date), application use (i.e., what applications were launched, when, and for how long), and mobile phone camera use (i.e., the time/date a picture was taken). All collected sensitive personal data, such as contact details (names, phone numbers), may be anonymized during data collection by the application through the built-in cryptographic hash functions of the Funf framework. At set intervals during each day, and only when participants' mobile phones were connected to Wi-Fi, the app sends collected data over the Internet to a remote central data server, in chunks of approximately five to ten megabytes (MB) per data file. Additional data may also include global positioning system (GPS) location data and accelerometer data.

In accordance with some embodiment of the system 10, the data collected by the mobile phone 70 may be sent over WiFi to the internet (or by using cellular network data transmission protocols) to a remote central data server for cloud processing and/or data logging. Data resulting from such remote processing and logging may be accessed by the mobile phone 70 or by the laptop 9 and may be used for computing values such as a mood index MX, and/or a modulation index MI, or other values required for the operation of the methods as disclosed in detail hereinafter. Alternatively, the processing and/or computations may be offloaded to the cloud remote server that may communicate any computed values (such as, for example MX and/or MI disclosed in detail hereinafter) over the internet (using WiFi or cellular data transmission protocols, or any other suitable communication protocols) to the mobile phone 70 and/or to the laptop 9 for use and/or for telemetrically sending such values to the telemetry module 133 to be used by the processor/controller(s) 14.

### Data Preprocessing and Feature Engineering

As disclosed in detail in the article by Joost Asselbergs et al (2016) cited in the reference list hereinbelow, raw EMA and unobtrusive EMA data may be preprocessed into a data file that summarized each day of each participant in a row of 53 variables.

### Prediction Targets: Ecological Momentary Assessment Mood

As in the LiKamWa et al. study, EMA data (i.e., both the one-dimensional mood measure and the two measures of the circumplex model, valence and arousal) are aggregated to daily averages as targets for the mood prediction algorithms. Daily averages are standardized within each participant (i.e., using means and standard deviations calculated for each participant separately).

### Mood Prediction Feature Set

Raw unobtrusive EMA data are aggregated into daily summaries and from these daily summaries the feature set may be derived as disclosed in detail in Table 1 of the Asselbergs et al. article cited in the reference list hereinafter.

For phone calls and SMS text messages, the number of interactions participants had with their five most frequent contacts are counted. Following LiKamWa et al, a histogram of this interaction frequency over a 3-day history window may be created and the normalized frequency count may be used as samples in the feature table. Similarly, a normalized 3-day histogram of call durations with the top five contacts may be created. Most participants interact only incidentally with persons outside their top five through calls or SMS text messages. Altogether, raw call/SMS text message data are summarized into three predictive features (top five call frequency and duration and top five contact SMS text message frequency), comprising 15 variables.

Raw mobile phone screen on/off events are transformed into two features: (1) the total number of times the screen is turned on per day and (2) the total amount of screen time per day (calculated as the differences between the times of the screen on/off events). Both features are transformed to standard normal variables within each participant.

Accelerometer data represents the acceleration of the smartphone on the x, y, and z planes. Acceleration is sampled for 5 seconds each minute (at sample frequencies estimated to vary from 20-200 Hz, as determined by the hardware and software characteristics of participants' mobile phones). Raw data are summarized (on the phone through Funf's Activity Probe) into a high activity variable by calculating the percentage of time at which the summed variance of the device's acceleration (on the x, y, z planes) was above a set “high activity” threshold (i.e., in which the summed variance exceeded 10 m/s²). These percentages are aggregated to the day level to provide an approximate measure of daily activity.

As daily measures of mobile phone application use, two 3-day normalized histograms for the daily frequency and duration of the five most frequently used mobile phone apps are created. In addition, normalized histograms of frequency and duration of the use of application categories are created. In accordance with the LiKamWa et al. study, applications as either built-in, communication, entertainment, finance, games, office, social, travel, utilities, other, or unknown (11 categories altogether) are categorized. Categories of logged applications are determined through a scripted query of the Google Play Store. Applications that are unknown to the Google Play Store were manually categorized on the basis of an Internet search. In sum, the final dataset consists of four features based on application usage logs: top five applications frequency, top five applications duration, application category frequency (11 categories), and application category duration (11 categories). These features result in 32 variables (5+5+11+11).

Mobile Phone camera logs are summarized to the number of photos taken per day. Next, this summary is transformed to the 0-1 scale for each participant separately by dividing all values by the maximum number of photos taken.

Finally, similarly to LiKamWa et al, the predictive feature set with a simple representation of mood history, by adding lag 1 and lag 2 transformations of each mood variable (standardized within each participant) is extended.

In total, a 53-dimensional variable set is derived from thirteen distinctive predictive features. Because regression models are sensitive to large differences in the scales of independent variables, the scales of the variables are transformed to the standard normal distribution (i.e., 99.7% of values ranging between −3 and 3). Interrelated variables (e.g., top 5 call and top 5 application use) are normalized to the 0-1 range, following the methods of LiKamWa et al.

As therapeutic brain stimulation is delivered, the results of this stimulation may also be periodically interrogated with ecological momentary mood assessments (EMA) to determine the impact of the stimulation on the reported mood and the resultant patient physiology. Based on mood, reporting and physiologic parameters, the stimulation parameters may also evolve and change. This could include changes in amplitude of stimulation, stimulating pulse width, and pulse frequency. The end result is a dynamic recording and stimulating system that continually self assesses performance based on the patient's reporting. Thus, this will enable biomarkers to not only be patient specific, but also to adjust over time should the patient's baseline physiology be non-stationary or should their fundamental brain states and physiologies change over time.

### Methods and Sensors for Determining Additional Depression Biomarkers

It is noted that the (optional) auxiliary sensors 15 (of FIG. 1) may optionally provide additional biomarkers that may be used as data useable, in some embodiments of the methods of the present application, for computing a global mood index (such as, for example the mood index MX. The sensor data may be sensed by the auxiliary sensor unit(s) 15 and may include heart rate (HR), perspiration data, pupil size (and or the temporal parameters of pupil size change when a test is presented to the patient, etc. as disclosed hereinabove

For example, it has been shown in the following article that in patients with major depression, there were significantly lower values of heart's beat-to-beat intervals and of the high-frequency peak of spectral analysis than in a normal (control) group.
Rechlin T, Weis M, Spitzer A, Kaschka W. P., “Are affective disorders associated with alterations of heart rate variability?” Journal of Affective Disorders 32 (I 994) 271-275.

It has also been shown in the flowing article that children with major depression had diminished late pupil dilation relative to comparison children, 9-12 seconds after a negative word was presented. Diminished late pupil dilation to negative word presentation was associated with higher levels of negative affect and a lower level of positive affect in the natural environment. Jennifer S. Silk, Ronald E. Dahl, Neal D. Ryan, Erika E. Forbes, David A. Axelson, Boris Birmaher, and Greg J. Siegle, “Pupillary Reactivity to Emotional Information in Child and Adolescent Depression: Links to Clinical and Ecological Measures”, IEEE Transactions on Affective Computing, Vol. 7, Issue: 1, (2016).

It is also known that Reduced frequency range in vowel production is a well documented speech characteristic of individuals with psychological and neurological disorders, and that Affective disorders such as depression and post-traumatic stress disorder (PTSD) are known to influence motor control and in particular speech production.

For example, in the following article the authors use an automatic unsupervised machine learning based approach to assess a speaker's vowel space. Experiments based on recordings of 253 individuals show a significantly reduced vowel space in subjects that scored positively on the questionnaires. The reduced vowel space for subjects with symptoms of depression can be explained by the common condition of psychomotor retardation influencing articulation and motor control.

Stefan Scherer, Gale M. Lucas, Jonathan Gratch, Albert “Skip” Rizzo, and Louis-Philippe Morency, “Self-Reported Symptoms of Depression and PTSD Are Associated with Reduced Vowel Space in Screening Interviews”, IEEE TRANSACTIONS ON AFFECTIVE COMPUTING, VOL. 7, NO. 1, pp. 59-72 (2016).

Such physiological parameters correlated with effects of depression or other mood disorders may be used in accordance with some embodiments of the systems and methods of the present application as additional (sensor based) biomarkers for assessing the mood of a patient.

For example, in some embodiments of the system 10, a heart rate (HR) sensor (either included in the mobile phone 70, or a separate HR sensor connectable to the mobile phone 70 or attached to the body of the patient) may be used to determine the patient's heart rate and provide the mobile phone 70 with heart rate data.

In another example, in some embodiments of the system 10, an external microphone or the microphone of the mobile phone 70 may be used to perform voice spectral analysis on the patient's voice (recorded while the patient is talking on the mobile phone 70). The recorded data may then be processed (For example, by the processor of the mobile phone 70 or in the cloud 31).

In another example, in some embodiments of the system 10, the size of the patient's pupil may be monitored and recorded either by a suitable application on the mobile phone 70 or by a separate device such as, for example, the AR headset 11 or a dedicated pupilometer worn by the patient and having a pupil size measuring capabilities may be used for obtaining pupil size data (and optionally eye tracking data) either periodically or in response to a test presented to the patient (such as the negative/neutral/positive word presentation test described by silk et al. hereinabove). Briefly, a test period may be initiated using the mobile phone 70 in which test words with different negative/neutral/positive emotional connotations are presented on the screen of the mobile phone 70 while the temporal variations of pupil size responsive to the presented word stimuli are measured and recorded either by the phone's front facing camera or by a dedicated pupilometer device worn by the patient.

It is noted that the methods of obtaining EMA data may also include an unobtrusive method of monitoring the patient's pupillary size variations responsive to words with negative emotional content while the patient is browsing web content. For example if the patient is browsing web content using the AR headset 11, the eye tracking function of the AR headset 11 may enable the system to identify the word which the patient is currently viewing and the pupil size determining function of the AR headset 11 may monitor the pupil size changes due to reading negative words to detect if the patient is in a depressive mood. The word(s) on which the patient looks may be identified as having normal (neutral) or negative emotional connotation based on a lookup table (LUT) stored in a memory or another storage device (on the AR headset 11, or on the laptop 9 or on the mobile phone 70 or on a remote server on the cloud 31).

Such a word lookup table may include a relatively small number of words (Typically, in the range of several tens to several thousands of words) to speed up word identification. If a word is identified (using the LUT) as having negative emotional connotations, the system may store the pupil size data recorded in a time period beginning a short time before the time the patient looked at the word and ending several seconds (typically 10-15 seconds) after the patient started looking at the word. The stored data may then be processed to determine if the parameters of the pupil's response are indicative of a depressed mood as disclosed in detail hereinabove and in the Silk et al. (2016) article cited above. The advantage of this method of obtaining mood related data via pupilometry is that the method is completely unobtrusive and eliminates the need to intrusively present a test session to the patient.

The data representing the parameters of the pupil's response may be processed to obtain parameters correlated with patient's mood (such as, for example, the amplitude of the late pupil dilation in response to the presentation of a negative word, the response latency and duration, or other pupil size characteristics. These parameters may be processed by the system 10 to assess the patient's mood. Care should be taken to assess each patient individually in a test period for determining the individual's pupil size variation dynamics because the pupil's response characteristics to negative word presentation may vary with patient's age and may be different in children, adolescents and adults (as described by Silk at al.) After the test results are obtained statistical analysis may determine the response parameters associated with depressive mood severity (as assessed by EMA). Such parameters may then be included in the model.

An example of a pupilometer that may be used in such pupil size determination is the Tobii Pro 2 wearable eye-tracker commercially available from Tobii A B, Stockholm, Sweden.

It is noted that the above three examples (HR measurements, pupil size dynamics measurements and vowel space measurements) are just three non-limiting examples of a biomarkers that may allow for a “multi-modal analysis” to establish the “model” disclosed in the present application. Such biomarkers may include any other measurable physiological and/or behavioral characteristics of a patient that exhibits a correlation with the patient's mood, any such biomarkers may be included in the data processing performed by the methods and algorithms for computation of the value of the mood index (MX) as disclosed herein. For example, the pupillary dynamics change test may be modified by replacing the negative word presentation by presenting images having a negative, neutral or positive connotation to the patient and monitoring the parameters of pupil size changes in response to the presentation of such images.

In some embodiments, the presentation of the images (or words) and the monitoring of pupil size changes may be performed by the AR headset 11 which may be used for image (or word) presentation and for determining pupil size changes. In other embodiments, the images (or words) may be presented on the screen of the mobile phone 70 or on the screen of the laptop 9 while the pupil size changes may be monitored by a dedicated pupilometer (such as, for example the Tobii pro 2, as disclosed herein) or by the AR headset 11.

The use of the term “model” relates to recording multiple various biomarkers (brain activity, heart rate, pupil dilation, voice spectrogram, or any other relevant mood indicative biomarkers), manual user input (e.g. typing in how they feel at the moment) and caretaker input, processing such multiple inputs using various algorithms to deliver a specific brain stimulation therapeutic paradigm and/or to provide visual/auditory feedback to either the user or his caretaker.

### Digital Signal Processing

Signals recorded from the system are processed in the following fashion. Channels with abnormal amplitude (e.g. >±1000 mV) or power spectra (e.g. harmonic noise) are flagged and removed from further analysis. The system performs spectral decomposition using Morlet wavelet convolution and estimated phase and amplitude envelopes from the resulting complex signals. All signals are then down-sampled to 300 Hz. All wavelet-derived properties (i.e. phase, amplitude and power) are generated from the whole signal, before trials are extracted, to avoid edge effects.

### First Method—Phase Amplitude Coupling (PAC) As Signal for Mood Biomarker.

Two sets of wavelet libraries are used for phase amplitude coupling (PAC). These libraries are created to satisfy mathematical constraints on phase-amplitude coupling measurements. Specifically, the bandwidth of the frequency-for-amplitude (Fₐ) must be twice the frequency-for-phase (Fₚ) of interest. The two wavelet libraries were constructed as follows.

Frequency for amplitude wavelets:

The full width at half-maximum (FWHM) of the Morlet wavelet as a lower bound estimate for bandwidth is used. Fₐ wavelets are designed to have a FWHM of 20 Hz and used 21 wavelets with center frequencies ranging from 20 Hz to 150 Hz in 5 Hz increments.

Frequency for phase wavelets: Narrow-band Fₚ wavelets are designed for phase specificity. Higher frequency resolution is employed for phase signals to distinguish between delta, theta and alpha rhythms. We used 20 Fₚ wavelets ranging from 1 Hz to 20 Hz with 1 Hz spacing and FWHM of 0.8 Hz.

Quantifying Phase-Amplitude Coupling (PAC) with the Modulation Index

PAC is measured using the modulation index (MI), which quantifies the magnitude of coupling. MI also provides a common measurement to compare different forms of PAC (e.g. unimodal versus bimodal) across different frequencies. MI is calculated as the Kullback-Leibler divergence between the uniform distribution (i.e. pure entropy) and the observed probability density P(j), which describes the normalized mean amplitude at a given binned phase (see P(j) below). Pairwise calculation of MIs for two sequences of frequencies produces a comodulogram. MI is calculated as follows:

$$MI = \frac{{D}_{KL} ( P , Q )}{log ( N )} \qquad ( 1 )$$

$${D}_{KL} ( P , Q ) = {\sum }_{j = 1}^{N} P ( j ) log ( \frac{P ( j )}{Q ( j )} ) \qquad ( 2 )$$

Where D_{KL} is the Kullback-Leibler divergence, P is the observed phase-amplitude probability density function, Q is the uniform distribution and N is the number of phase bins. P follows the equation:

$$P ( j ) = \frac{{〈 {A}_{{f}_{A}} 〉}_{{ϕ}_{{f}_{P}}} ( j )}{{\sum }_{k = 1}^{N} {〈 {A}_{{f}_{A}} 〉}_{{ϕ}_{{f}_{P}}} ( k )} \qquad ( 3 )$$

where [symbol] A_{f}_{_{A}} [symbol] _{ϕf}_{ₚ}(j) is the mean f_{A} amplitude signal at phase bin j of the phase signal ϕ_{f}_{ₚ}. Phase is divided into 18 bins of 20-degree intervals.

To identify PAC frequency pairs of interest, trials are sorted by EMA indicated mood and divided them into quartiles ranging from best to worst mood. We use signals from the highest and lowest mood measurement quartiles to generate P(j) distributions of normalized amplitude per binned phase, from which the MI is calculated.

### Statistical Analysis

### Band Limited Power and PAC Time Series Comparisons:

Cluster candidates were generated using t-statistics to test the null hypothesis that there was no difference between categories at each sample. If a sample t-statistic exceeded an alpha level of 5% then the null hypothesis was rejected for the sample and it was considered a cluster candidate. Temporally adjacent cluster candidates are grouped into a single cluster and their t-statistics are summed to produce a clustering statistic. The clustering statistic of the observed data were tested against a permutation distribution. To produce the permutation distribution, trial labels (e.g. valid vs. invalid) are shuffled and randomly reassigned 10,000 times. For each shuffle, cluster candidates and clustering statistics are generated as described above. The maximum clustering statistic from each shuffle are used to create the permutation distribution. P-values are calculated for observed clusters using the formula P=(r+1)/(n+1), where r is the number of shuffled clustering statistics greater than the observed clustering statistic and n is the total number of shuffled sets used. Multiple comparisons are corrected across cortical sites with the False Discovery Rate (FDR) correction method.

### Phase-Amplitude Coupling Comparison:

A two-dimensional non-parametric permutation test is adapted to make cluster-based statistical inferences on comodulograms based on the difference between positive and negative mood trials. First, 1,500 shuffled distributions are generated for each cortical site by randomly reassigning mood measurements to trials, sorting, dividing into quartiles, and calculating the absolute difference in comodulograms for elevated and depressed mood quartiles as follows:

$${d}_{{f}_{A} {f}_{P}} = \vert {MI}_{{f}_{A} {f}_{P}}^{fast} - {MI}_{{f}_{A} {f}_{P}}^{slow} \vert \qquad ( 4 )$$

The pooled variance in each frequency pair in the distribution of d_{f}_{_{A}}_{f}_{_{P}}^{shuffled} is used to determine the cutoff threshold specific to each frequency pair. Adjacent supra-threshold frequency-pairs are grouped together in clusters and t-statistics are summed. The null hypothesis is tested that the shuffled data was no different from the observed data using a two-dimensional cluster based permutation test where diagonals are not considered neighbors.

PAC time series are calculated using MI calculations in a 500 ms sliding window with 50 ms increments. Differences between PAC time-series for mood categories are calculated with the one-dimensional cluster-based permutation test described above.

### Second Method: Amplitude Modulation.

A second method is of identifying mood related physiological biomarkers involves assessing amplitude changes at specific frequencies. Using the method described above amplitude changes can also be determined to correlate with a mood state. This may be done for different amplitudes at a single electrode at different frequencies, or different frequency amplitudes at different electrode locations.

Method of Using Amplitude Changes for determining Mood Index

Raw signals were high-pass filtered at 0.05 Hz using a 3^{rd} order Butterworth filter. Electrodes containing an excessive amount of noise are removed from further analysis. Additionally, time epochs containing artifact in a majority of electrodes are discarded. The mean of the non-noisy electrodes are regressed out of the signal from each electrode.

The power spectral density (PSD) of the cortical signal from each electrode are estimated using Welch's method. The Welch's windows had a width of 2 seconds (frequency resolution of 0.5 Hz) and a 50% overlap. Power spectra are consolidated into canonical frequency bands (delta frequency band: 0.1-4 Hz, theta frequency band: 4.5-8 Hz, alpha frequency band: 8.5-12 Hz, sigma frequency band: 12.5-15 Hz, beta frequency band: 15.5-25 Hz, low gamma frequency band: 25.5-50 Hz, and high gamma frequency band: 70-110 Hz) and then normalized by the total power across all frequency bands.

### Spatio-Spectral Differences Between States

Differences in cortical electrophysiology between the elevated mood and the depressed mood state (as defined by EMA) are examined for each subject in the frequency domain using the sensitivity (or discriminability) index d′_{b,c} from signal detection theory:

$${d}_{b , c}^{′} = \frac{{μ}_{b , c , depressed} - {μ}_{b , c , not depressed}}{\sqrt{{ρ}_{depressed} {σ}_{b , c , depressed}^{2} + {ρ}_{depressed} {σ}_{b , c , not depressed}^{2}}} \qquad ( 5 )$$

where μ_{b,c} and σ_{b,c} are the mean band limited power (BLP) and the standard deviation of the BLP, respectively, across all epochs for the specified cognitive state at frequency band b and electrode c. ρ is the proportion of data belonging to each class.

Logistic regression model for state estimation A logistic regression is employed to build models that could accurately predict the mood states given the cortical signals. The cortical signals from each behavioral epoch are broken into 120 second non-overlapping segments or instances. The PSD is calculated for each instance and consolidated into frequency bands resulting in a set of features, p ∈ [symbol] ^{C×B} where C is the number of electrodes and B is the number of frequency bands. The features and the class labels, y⁽ⁱ⁾ (−1 for depressed or +1 for not-depressed) for all instances from a particular epoch are randomly placed as a group into either a training or a test set such that class distribution is preserved in each set and so approximately 80% of the total number of instances across all epochs are in the training set (approximately 20% in the test set). Five-fold cross validation is used to learn the models. Each fold had a unique test set.

Within a fold, each feature is centered by the feature mean across all training instances and normalized by the Euclidean norm of the feature across all training instances:

$${x}_{b , c}^{( i )} = \frac{{p}_{b , c}^{( i )} - {\overline{p}}_{b , c}}{\sqrt{{\sum }_{i = 1}^{n} {( {p}_{b , c}^{( i )} - {\overline{p}}_{b , c} )}^{2}}} , \qquad ( 6 )$$

Where λ⁽ⁱ⁾_{b,c} is the centered normalized feature mean, p_{b,c}⁽ⁱ⁾ is the BLP of instance i at frequency band b and electrode c, and p̅_{b,c} is the average BLP over the training set within a fold. The feature mean and norm calculated from the training set are also used to center and normalize the test set within the fold.

Models are learned using all features, x ∈ [symbol] ^{C×B} and also using a subset of features, x_{b} ∈ [symbol] ^{C×1}, i.e., a unique model is learned for the group of features belonging to each frequency band. For each training set, R_{b}={x_{b}⁽¹⁾, . . . , x_{b}⁽ⁿ⁾; y⁽¹⁾, . . . , y⁽ⁿ⁾} or R={x⁽¹⁾, . . . , x⁽ⁿ⁾; y⁽¹⁾, . . . , y⁽ⁿ⁾} of n instances for a single patient, the system models the probability that a patient was in the depressed or non-depressed state for instance i using a linear model transformed by the sigmoid function, commonly referred to as logistic regression:

$$P r ( {y}^{( i )} \vert {z}^{( i )} ; w ) = \frac{1}{1 + {e}^{- {y}^{( i )} {w}^{T} {z}^{( i )}}} , \qquad ( 7 )$$

where z is either x or x_{b}, and w is a weight vector that parameterizes our model. The system solves for these weights by maximizing the probability that each reading was predicted correctly:

$${max}_{w} {\prod }_{i = 1}^{n} P r ( {y}^{( i )} \vert {z}^{( i )} ; w ) , \qquad ( 8 )$$

or, equivalently, by minimizing the sum across instances of the negative logarithm of the probability:

$${min}_{w} {\sum }_{i = 1}^{n} log ( 1 + {e}^{- {y}^{( i )} {w}^{T} {z}^{( i )}} ) . \qquad ( 9 )$$

Modeling the probability allows prediction uncertainty to be represented naturally, which has practical value for BCI applications because it is safer if the BCI remains off when the system is uncertain of the user's cognitive state.

### Identification of Optimal Cortical Locations

Optimal electrode locations for estimating the mood states are identified by constraining the optimization problem. By adding an [symbol] ₁/ [symbol] ₂ mixed-norm of the feature weights, the system is forced to converge on a solution that uses BLP from all frequency bands, but from a sparse set of electrodes. The [symbol] ₁/ [symbol] ₂ mixed-norm regularized logistic regression is shown below:

$${min}_{w} {\sum }_{i = 1}^{n} log ( 1 + {e}^{- {y}^{( i )} {w}^{T} {x}^{( i )}} ) + λ {\sum }_{c = 1}^{C} \sqrt{{\sum }_{b = 1}^{B} {( {w}_{b , c} )}^{2}} , \qquad ( 10 )$$

where λ≥0 trades off prediction accuracy on the training set with electrode weight sparsity. Similarly, for the models only utilizing features from one specific frequency band as an input, x_{b}, the system employs an [symbol] ₁-regularized logistic regression model,

$${min}_{w} {\sum }_{i = 1}^{n} log ( 1 + {e}^{- {y}^{( i )} {w}^{T} {x}_{b}^{( i )}} ) + λ {‖ w ‖}_{1} . \qquad ( 11 )$$

Electrode sparsity is independently varied from one to four electrodes (or more electrodes if needed). The corresponding hyper-parameter λ is learned using a binary search on the training set of each fold. Initially, an arbitrary value is assigned to λ, and a subsequent model is constructed. If the model is more sparse than desired, then λ is decreased to reduce the impact of the constraint on the model. Conversely, if the model is less sparse than desired, then λ is increased. This process is systematically repeated until λ converged on a value that provided the desired electrode sparsity in the model.

### Model Prediction and Performance

The output of each model is the probability that the subject was in the depressed state (Equation (7) above; y⁽ⁱ⁾=1). Thus, the state is estimated using the following rule:

$$State Estimation ( i ) = \{ \begin{matrix}depression ; & Pr ( {y}^{( i )} = 1 \vert {x}^{( i )} ; w ) \geq 0.5 \\ not depressed ; & Pr ( {y}^{( i )} = 1 \vert {x}^{( i )} ; w ) < 0.5\end{matrix} \qquad ( 12 )$$

Model performance is quantified by evaluating the accuracy, sensitivity, and specificity on the test set of each fold.

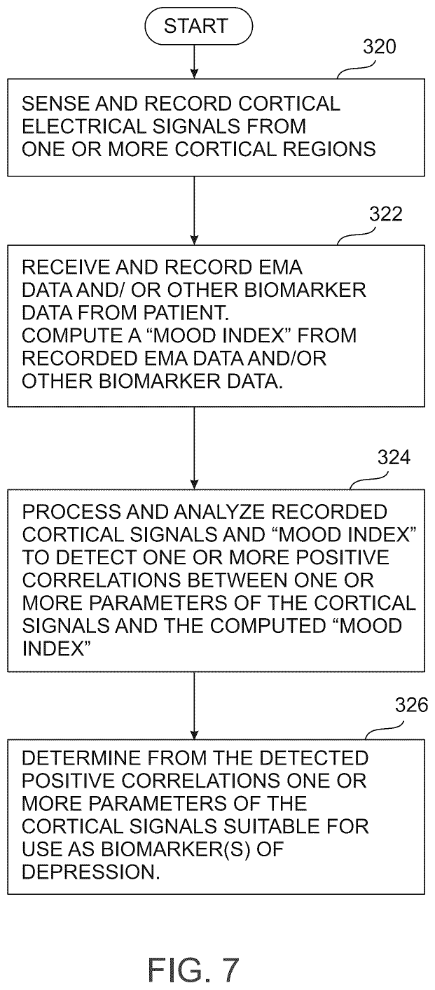

Reference is now made to FIG. 6 which is a schematic flow chart diagram illustrating the steps of a method for delivering brain stimulation therapy by processing sensed cortical activity and ecological momentary mood assessment data of a patient, in accordance with some embodiments of the methods of the present application.

The system (such as, for example the system 10) starts, and senses (and records) cortical electrical signals (step 300). The cortical region may be the right DLPFC, the left DLPFC, both the left and the right DLPFC or any other region of the PFC. The system 10 processes the recorded cortical signals (step 302). The system then checks if a biomarker for depression was detected in the recorded signals (step 304). The marker may be the modulation index MI or any other suitable cortical biomarker (such as, for example, the state estimation wherein the probability that the patient is depressed is equal to or greater than 0.5 as disclosed in detail hereinabove. If no biomarker is detected (such as, for example, if the probability that the patient is depressed is smaller than 0.5) the system return control to step 300 and continues to sense and process the cortical signals. If the biomarker was detected the system then checks if the currently computed value of the mood index MX is equal to or smaller than a threshold value (step 306). The value of threshold may be determined in a test period or may be set by the caretaker or physician. In accordance with some embodiments, the mood index value may be calculated as follows:

$$MX = ( a {A}_{1} + b {B}_{2} + c {C}_{3} + … + {mM}_{n} ) / n$$

Wherein,
n is the total number of biomarker parameters used (including the cortical signal biomarker and/or one or more of the biomarker parameter values as sensed by the auxiliary Sensor(s) 15).
a, b, c, . . . m are n weighing factors
and A, B, C, . . . M are the actual biomarker values normalized to a range of 1-10 on the basis of correlation with patient reported EMA data.

If the value of the mood index (MX) is larger than the threshold value, the system transfers control to step 300. If the value of the mood index (MX) is equal to or smaller than the threshold value the system delivers cortical stimulation (step 308). The stimulation may be delivered to the right DLPFC and/or to the left DLPFC and/or to any other selected region of the PFC. The system then checks if the biomarker for depression is still detected (step 310). If the biomarker for depression is still detected, the system transfers control to step 308 to continue cortical stimulation. If the biomarker for depression is not detected, the system checks if the value of the mood index MX is larger than the threshold value (step 312). If the value of MX is larger than the threshold value, the system terminates stimulation (step 314) and returns control to step 300. If the value of MX is not larger than the threshold value the system transfers control to step 308 to continue delivering the cortical stimulation.

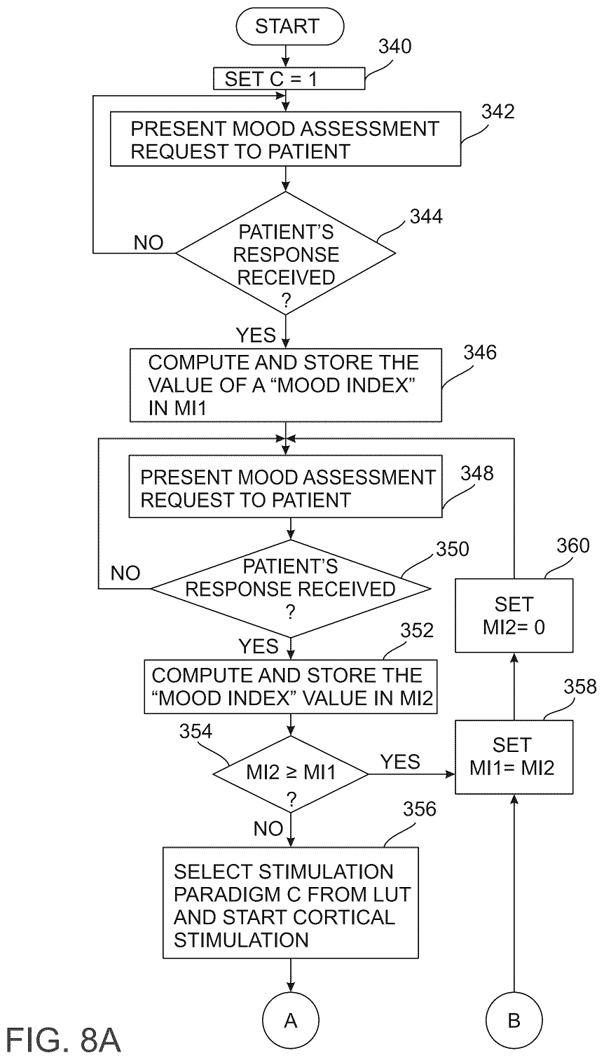

Reference is now made to FIG. 7 which is a schematic flow chart diagram illustrating the steps of a method for assessing the correlation between one or more parameters of recorded cortical signals and a Mood index computed from ecological momentary mood assessment (EMA) data of a patient, in accordance with some embodiments of the methods of the present application.

The testing method includes sensing and recording cortical signals from one or more cortical regions (step 320). The cortical regions being sensed may include the right DLPFC and/or to the left DLPFC and/or any other selected region of the PFC.

The system receives and records EMA data and/or other biomarker data from the patient (such as, for example, biomarkers sensed by any of the auxiliary sensor(s) 15) and computes a mood index from the EMA data and/or other biomarker data (step 322).

The system may then process and analyze the recorded cortical signals and the mood index to detect one or more positive correlations between one or more parameters of the cortical signals and the computed mood index (step 324).

The system then determines from the detected positive correlations, one or more parameters of the cortical signals suitable for use as one or more biomarkers of depression (step 326).

It is noted that while it may be possible to use a single type of stimulation paradigm to deliver anti-depressive therapeutic treatment, in some embodiments of the methods the system may deliver graded stimulation paradigms as anti-depressive therapeutic treatment.

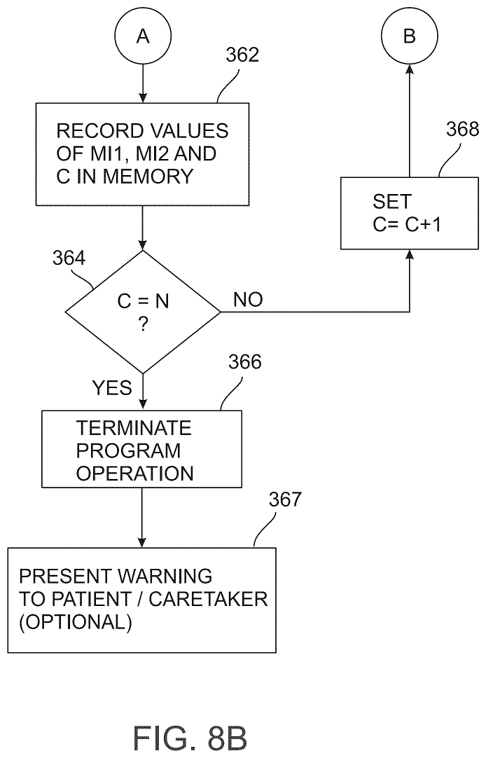

Reference is now made to FIGS. 8A-8B which are schematic flow chart diagrams illustrating the steps of a method for delivering graded brain stimulation therapy to a patient by processing sensed cortical activity and ecological momentary mood assessment data of the patient, in accordance with some embodiments of the methods of the present application.

The system may start by setting the value of a parameter C to zero (step 340). The system then presents a mood assessment request to the patient (step 342). The request may be in the form of a screen on the mobile phone 70 or the laptop 9 asking the patient to provide a mood self assessment representative of the patient's subjective feeling of whether he/she is depressed and the degree of depression. For example, in some embodiments of the methods, the patient may input a number in the range of one to ten where the number ten signifies the most severe depressed state and the number one signifies a completely non-depressed mood.

The system then checks if the patient response to the request has been received (step 344). If the patient's response was not received (within an allocated response time period (for example two minutes), the system returns control to step 342 to present the request again. If the patient's response was timely received within the allocated response time period the system computes and stores the value of the received self assessed mood index, computes the value of MX based on the modulation index MI, the EMA data and the patient's self assessment value in a parameter MI1 (step 346). After a preset time period (for example, two hours) the system presents another mood assessment request to the patient (step 348). The system then checks if a patient's response was received within the allocated response time period (step 350). If a patient's response was not received within the allocated response time period, the system returns control to step 348 for presenting the request again. If the patient's response was received The system then computes the value of MX based on the modulation index MI, the EMA data and the patient's new self assessment value and stores the computed value of MX in a parameter MI2 (step 352).

The system then checks if MI2≥MI1 (step 354). If MI2≥MI1 the system stores the value of MI2 in MI1 (step 358), sets the value of MI2 to zero (step 360) and transfers control to step 348. If MI2=MI1, the system selects a stimulation paradigm C from a look up table (LUT) including N graded stimulation paradigms and starts cortical stimulation using stimulus paradigm C (step 356). The system records the values of MI1, MI2 and C in memory (step 362) for providing a caretaker with logged stimulus history information. The system then checks if the parameter C=N. If C=N, the system terminates stimulation (step 366) and may optionally present a warning signal (visual or audible, such as, for example an audible sound or warning screen on the mobile phone 70 or on the laptop 9) to the patient and/or to the caretaker (step 367).

If C is not equal to N, the system sets the value of C to C+1 (step 368) and transfers control to step 358.

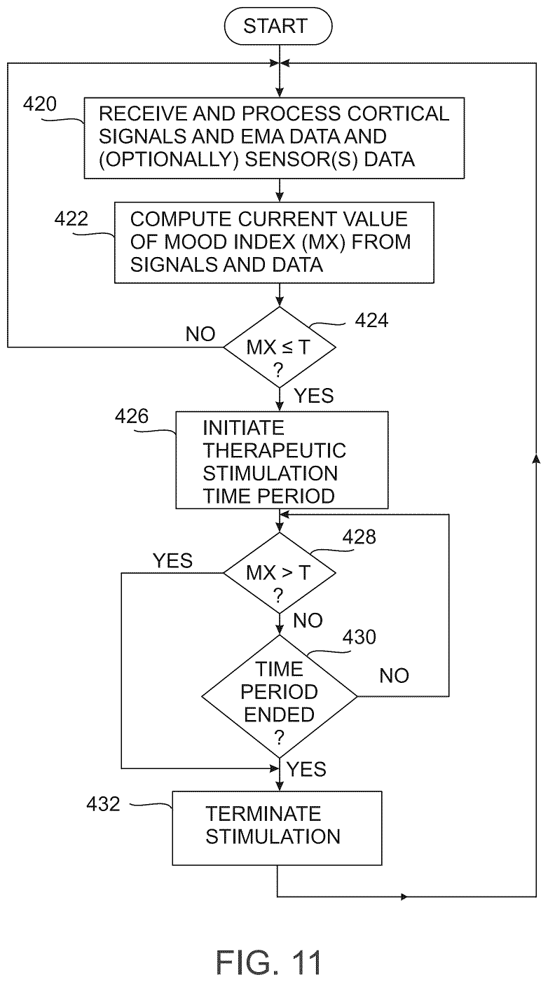

In the present method prior to operation the program operating on the system may be loaded with an LUT that includes N stimulation paradigms having graded increasing efficacy for treating depression as determined in a testing period assessing the efficacy of various different stimulation paradigms in treating depressive mood. For example, if the stimulation paradigm comprises delivering a train of supra-threshold stimulating pulses to the stimulated cortical region(s), the grading may be performed by using increasing pulse frequencies for different stimulation paradigms. In some embodiments the number and location of the electrodes from which stimulation is delivered may be changed, in some embodiments in which the implants allow for stimulation of deep brain structures, such as the systems 140 and 160 disclosed hereinafter (and illustrated in FIGS. 12-17) the graded efficacy stimulation paradigms may be achieved by changing the cortical region(s) being stimulated and/or the deep brain structure(s) being stimulated. For example, if it is experimentally found in a testing period that stimulating the right DLPFC is less efficacious than stimulating the right DLPFC and the anterior cingulate cortex and that stimulating the left DLPFC, the ventral caudate nucleus is even more effective in treating depressive mood, it may be possible to use such different stimulation paradigms for delivering graded stimuli paradigms in response to increasing severity of the patient's mood. Any suitable combinations and/or sub-combinations of such grading methods may be used in the method. For example it may be possible to change the number and location of the stimulating electrodes combined with changing stimulating pulse frequency and/or combined with changing the specific combination of the regions being stimulated.

In such methods, in some embodiments the system starts from the lowest efficacy stimulation paradigm (C=0) and if no alleviation of mood severity is detected the system will successively use more efficient stimulus paradigms until the most effective stimulus paradigm has been used at which time the system will stop stimulation and notify the patient and/or the caretaker. Alternatively, if the most highly effective stimulus paradigm has been used without successfully reducing the degree of depressive mood severity, the system may (optionally) reset C such that C=0 and begin a new cycle of graded stimulation (not shown in FIGS. 8A-8B).

It is noted that while as explained hereinabove, the modulation index MI may be computed using the spectral power at a multiplicity of different frequency bands, this is not obligatory, and some methods may use only the spectral power at a single selected frequency band.

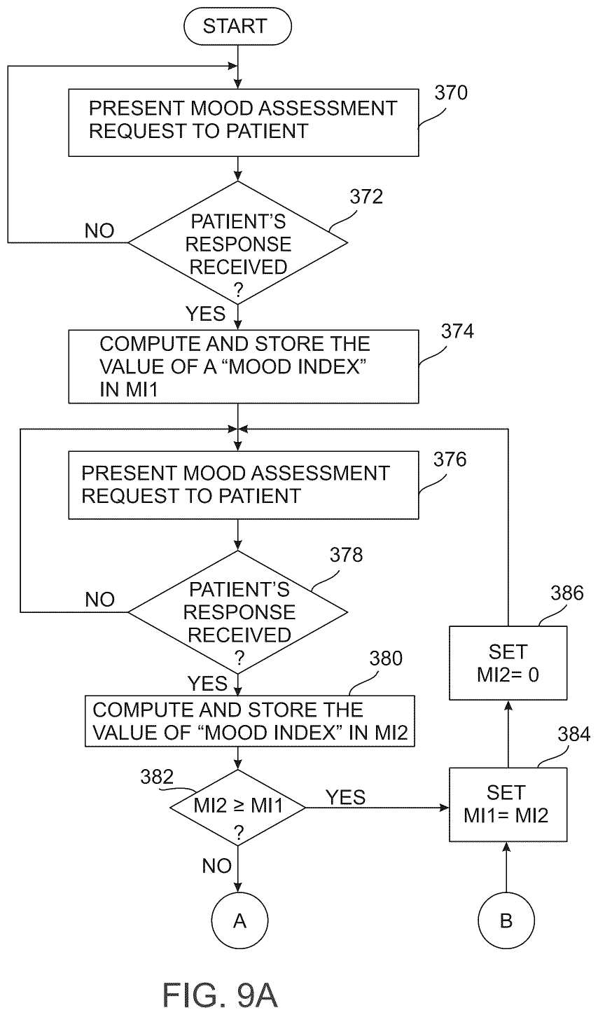

Reference is now made to FIGS. 9A-9B which are schematic flow chart diagrams illustrating the steps of a method for delivering brain stimulation therapy to a patient by using the value of the power at the gamma frequency band (P_{γ}) of sensed cortical signals and the ecological momentary mood assessment (EMA) data of the patient, in accordance with some embodiments of the methods of the present application.

In the method of FIGS. 9A-9B, the system then presents a mood assessment request to the patient (step 370). The request may be in the form of a screen on the mobile phone 70 or the laptop 9 asking the patient to provide a mood self assessment representative of the patient's subjective feeling of whether he/she is depressed and the degree of depression. For example, in some embodiments of the methods, the patient may input a number in the range of one to ten where the number ten signifies the most severe depressed state and the number one signifies a completely non-depressed mood.

The system then checks if the patient response to the request has been received (step 372). If the patient's response was not received (within an allocated response time period (for example three minutes), the system returns control to step 370 to present the request again. If the patient's response was timely received within the allocated response time period the system computes and stores the value of the received self assessed mood index, computes the value of MX based on the modulation index MI, the EMA data and the patient's self assessment value in a parameter MI1 (step 374). After a preset time period (for example, one hour) the system presents another mood assessment request to the patient (step 376). The system then checks if a patient's response was received within the allocated response time period (step 378). If a patient's response was not received within the allocated response time period, the system returns control to step 376 for presenting the request again. If the patient's response was received the system computes the value of MX based on the modulation index MI, the EMA data and the patient's new self assessment value and stores the computed value of MX in a parameter MI2 (step 380).

The system then checks if MI2≥MI1 (step 382). If MI2≥MI1 the system stores the value of MI2 in MI1 (step 384), sets the value of MI2 to zero (step 386) and transfers control to step 376. If MI2=MI1, the system senses signals in one or more cortical regions (step 388), performs fast Fourier transform (FFT) of the recorded cortical signals (step 390) and computes from the resulting power spectra the power at the gamma frequency band Pγ(step 392). The system then checks if Pγ≤Threshold (step 394). The threshold may be a preset threshold value determined in test period correlating the value of Pγ with EMA data and/or a self assessment of mood received from the patient.

If Pγ<Threshold, the systems start stimulating the target brain region(s) (step 396) and transfers control to step 384. The target brain regions for stimulation may be selected from any of the cortical regions disclosed in the present application and/or any of the deep brain structures disclosed in the present application, and/or any combination or sub-combination thereof, as disclosed hereinabove. If Pγ>Threshold, the system transfers control to step 388 to continue the sensing of cortical signals.

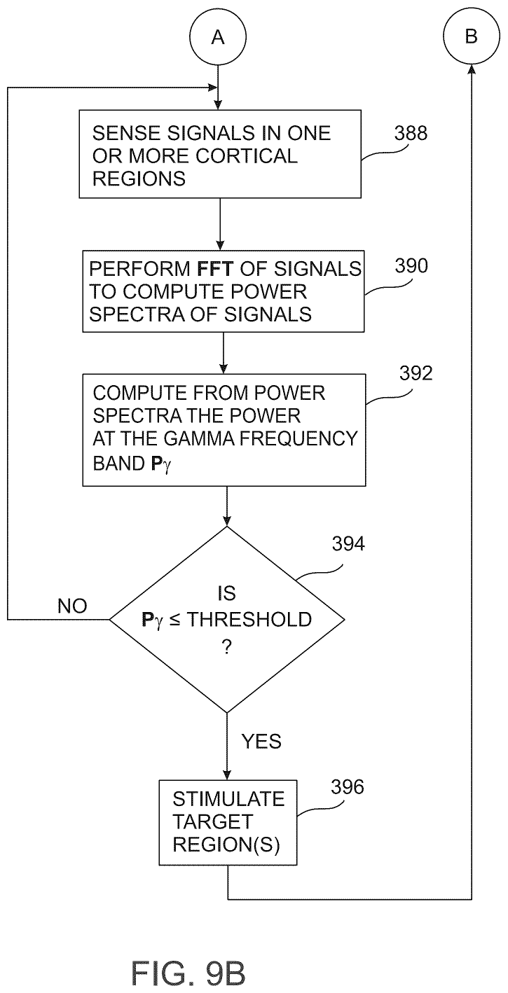

Reference is now made to FIG. 10 which is a schematic flow chart diagram of a method for delivering graded stimulation therapy to a patient responsive to processing cortical signals, EMA data and additional sensor data, in accordance with some embodiments of the methods of the present application.

The system starts by setting the value of the following parameters K=1 and N=n wherein K is a counter parameter and n is the number of available stimulation regimes SR_{K} (step 400). The system then initiates a stimulation regime SR_{K} (step 404). The system then receives cortical signals and EMA data and (optionally) sensor(s) data received from any of the auxiliary sensor(s) of the system (step 406). The system then computes the current value of the mood index MX from the currently available cortical signals and from the EMA data and/or (optionally) the sensor(s) data. The system then checks if MX≤T, wherein T is a threshold value determined in a suitable system test period directed at empirically finding an acceptable threshold, above which stimulation should be increased.

If MX >T, the system transfers control to step 406. If MX≤T, the system checks if K=n, indicating that the most effective stimulation regime has been used the system initiates an alarm signal to the patient and reports to the caretaker and/or patient (using audio or visual signals as described in detail hereinabove (step 414), sets the value of the counter K to K=1(step 416 and transfers control to step 404 for continues stimulation using the stimulation regime SR_{K}=SR₁. If K≠n, the system sets K=K+1 (step 418) and returns control to step 404.

In this method, there are n stimulation regimes that may be stored in a suitable LUT as disclosed hereinabove the stimulation regimes SR_{K} are arranged in an increasing efficiency of treating a depressive mood as n increases (where n is an integer number. Thus, SR₁, SR₂, SR₃, . . . , SRₙ are arranged in the order of increasing effectiveness as depressive mood therapy.

The stimulation regimes may be any of the different stimulation paradigms as disclosed hereinabove.

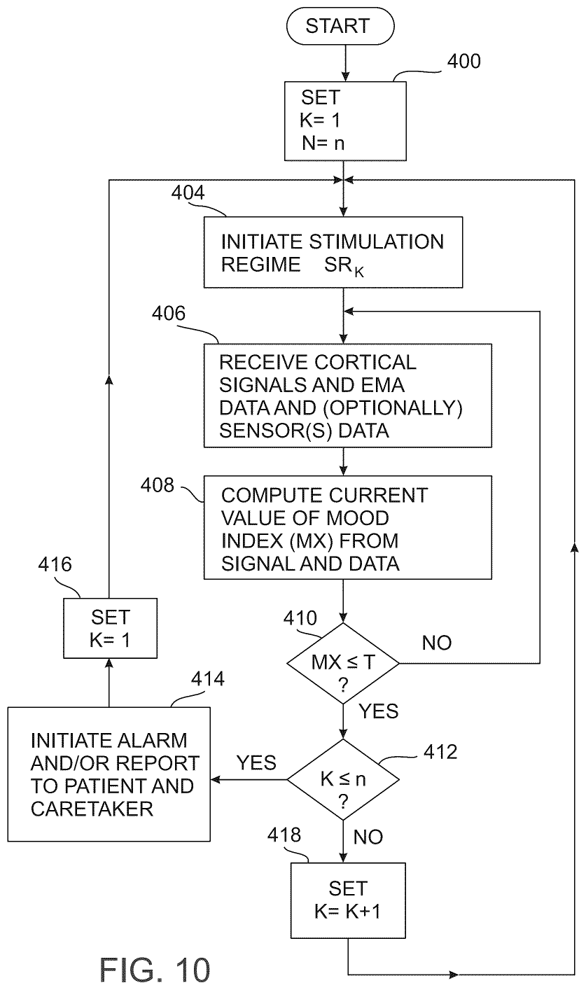

Reference is now made to FIG. 11 which is a schematic flow chart diagram of a method for delivering intermittent brain stimulation therapy to a patient responsive to processing cortical signals, EMA data and additional sensor data, in accordance with some embodiments of the methods of the present application.

The system starts and receives and processes cortical signals, EMA data and (optionally) sensor(s) data received from one or more of the auxiliary sensor(s) 15 of the system (step 420). The system then computes the current value of the mood index MX as computed from the sensed cortical signals and the EMA data, and (optionally the sensor(s)' data (step 422). The system then checks if MX≤T, wherein T is a preset threshold value as described hereinabove (step 424). If MX >T, the system transfers control to step 420. If MX≤T, the system initiates a therapeutic stimulation time period (step 426). The time period may be any suitable time period that may be empirically found (in a preliminary testing period conducted for each individual patient) to be sufficient to have a therapeutic effect on a depressed mood. Such a therapeutic stimulation time period may be in the range of several minutes to several hours, depending, inter alia, on the type of stimulation being delivered, the brain regions being stimulated and other stimulation parameters.

While the stimulation is being performed, the system checks if MX>T (step 428). If MT >T, the system terminates the stimulation (step 432) and transfers control to step 420. If MX≤T, the system checks if the therapeutic stimulation time period has ended (step 430). If the stimulation time period has not ended, the system returns control to step 426 (while continuing the stimulation). If the stimulation time period has ended, the system terminates the stimulation (step 432) returns control to step 428 and returns control to step 420.

It is noted that the method of FIG. 11 always uses the same stimulation type (which may be programmed by the caretaker before starting the operation of the method). The type of stimulation may be any of the stimulation type disclosed hereinabove in any suitable combination of stimulation target(s) but it is not modified or changed during the operation of the program or method except when it is terminated before the end of the therapeutic stimulation period due to the detection of the condition MT >T in step 428.

It is noted that the systems of the present application are not limited to stimulation of cortical regions (such as, the left and/or right DLPFC). In some embodiments, deep brain structures may also be stimulated as part of the therapeutic stimulation for treating mood disorders.

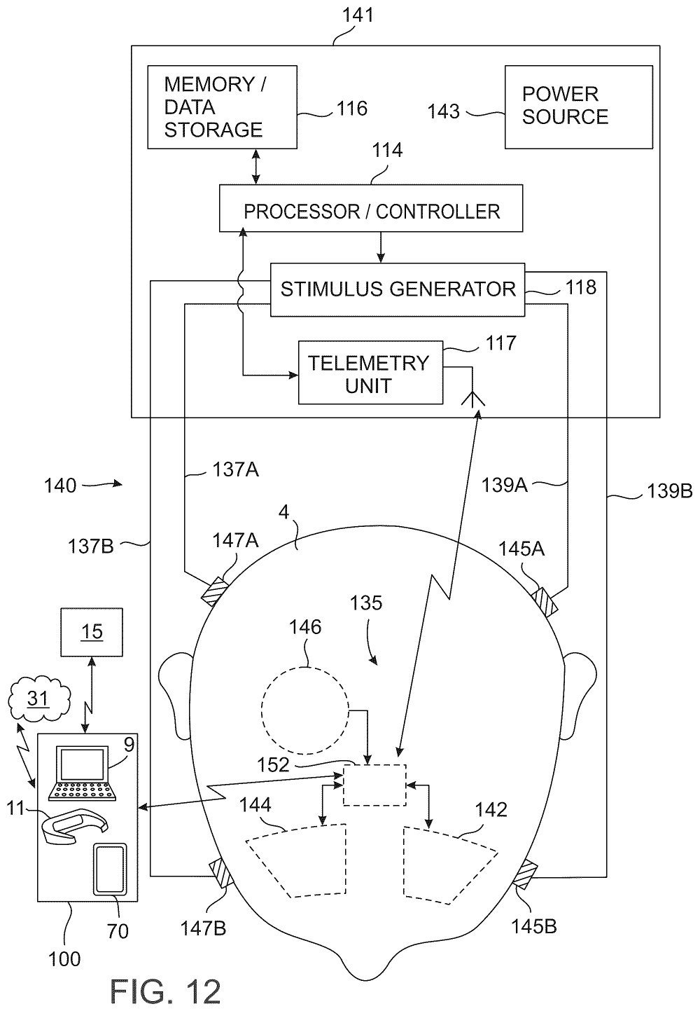

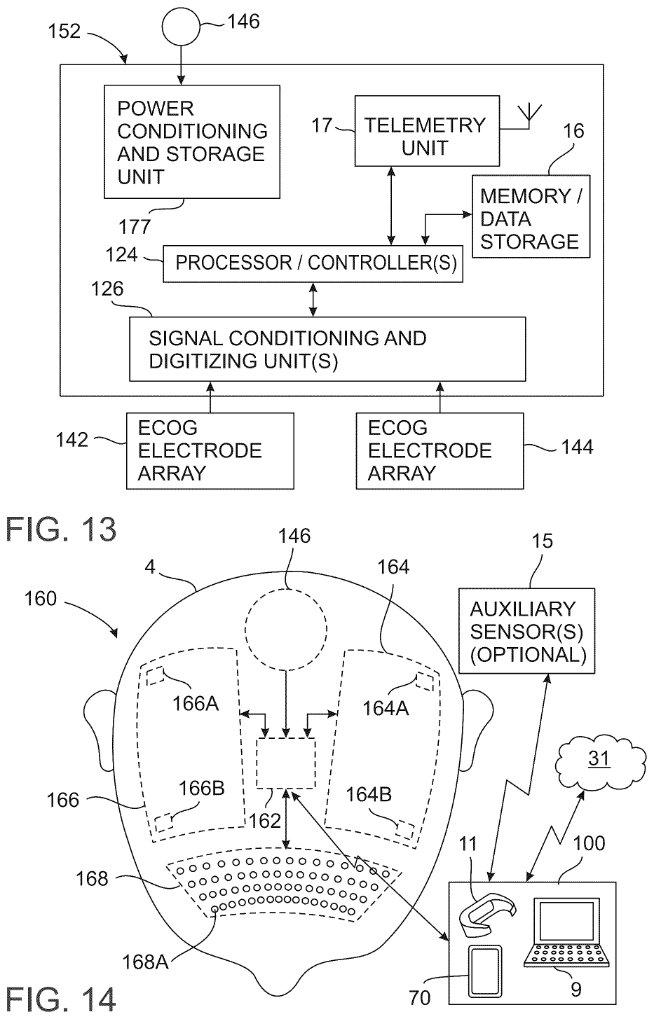

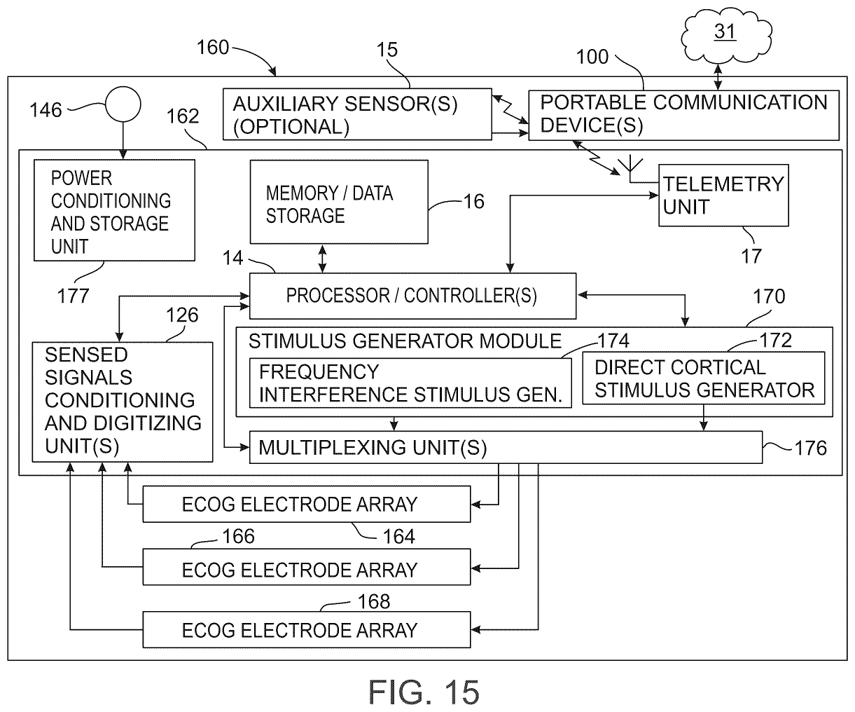

Reference is now made to FIGS. 12-15. FIG. 12 is a schematic block diagram illustrating a system for treating mood disorders including scalp electrodes for performing transcranial frequency interference stimulation of cortical and/or deep brain structures and intra-cranially implanted ECOG electrode arrays for sensing and/or stimulating one or more cortical regions, in accordance with some embodiments of the systems of the present application. FIG. 13 is a schematic block diagram illustrating the functional components of an intra-cranial part of the system of FIG. 12. FIG. 14 is a schematic drawing illustrating a system for treating a mood disorder having multiple intra-cranial ECOG arrays for performing sensing in one or more cortical regions and for performing trans-cranial frequency interference stimulation (TFIS) of one or more deep brain structures and/or direct stimulation of one or more cortical region(s), in accordance with some embodiments of the systems of the present application. FIG. 15 is a schematic functional block diagram illustrating functional components included in the system of FIG. 14.

Turning to FIG. 12, the system 140 includes an extra-cranial module 141 and an intra-cranial module 135 wirelessly in communication with each other. The extra-cranial module 141 also includes one or more processor/controller(s) 114 suitably coupled to a memory/data storage device 116. The extra-cranial module 141 also includes a power source 143 for energizing the components of the extra cranial module 141. The stimulus generator 118 is suitably electrically connected to four stimulating electrodes 145A, 145B, 147A and 147B that are attached to the surface of the skin of the head 4 of the user at four different positions. The stimulating electrodes 145A, 145B, 147A and 147B may be electrically coupled to the surface of the skin of the head 4 by using any suitable electrically conducting gel or paste (such as for example any EEG electrode gel or paste). The stimulating electrodes 145A, 145B, 147A and 147B are connected to the stimulus generator 118 by suitable electrically conducting insulated leads 139A, 139B, 137A and 137B, respectively. A first stimulating current at a first frequency f may be applied by the stimulus generator 118 to a first electrode pair 145A and 145B and a second stimulating current at a second frequency f+Δf may be applied by the stimulus generator 118 to a second electrode pair 147A and 147B. The two frequencies f and f+Δf are both in a frequency range too high to recruit neural firings (for example f and f+Δf ≥1 Khz). The stimulus generator 118 is suitably electrically connected to the processor/controller(s) 114 which controls the operation of the stimulus generator 118.

Due to the interference of the two different oscillating the electrical fields generated by the simultaneous stimulation through the first electrode pair 145A and 145B and the second electrode pair 147A and 147B at two different frequencies, selective neuronal activation may be achieved in deep brain structures that are located in a defined region where interference between the electric fields results in a prominent electrical field envelope modulated at the difference frequency Δf.

This selective stimulation method is referred to as trans-cranial interference (TI) stimulation and is described in detail in the paper by Grossman N. et al. referenced hereinafter and will also be interchangeably referred to as Non-invasive Temporal interference stimulation (NTIS) throughout the present application. The exact positioning of the electrodes on the head 4 of the user or patient and the stimulating intensity and frequencies may be determined, inter alia, by the position in the brain of the deep brain structure(s) that are being stimulated, the thickness and other physical and electrical parameters of the skull bones (which may significantly vary between different users of different ages) and may be empirically experimentally determined by suitable testing of each individual user/patient.

As the size and shape of the region of neuronal recruitment region in NTIS may be varied by adjusting or varying the positions of the stimulating electrodes 145A, 145B, 147A and 147B, and/or the stimulus frequency and intensity (amplitude) parameters, it is possible to stimulate one deep brain structure or several deep brain structures by suitably varying the size, shape and position of the neuronal recruitment region as disclosed in detail by Grossman et al.

The system 140 may also include the auxiliary sensor(s) 15, as disclosed in detail with reference to the system 10 of FIG. 1. The auxiliary sensor(s) 15 may wirelessly communicate with the wireless communication device(s) 100 (such as, for example with the mobile phone 70 and/or the laptop 9 and/or the AR headset 11).

The extra-cranial module 141 also includes a telemetry unit 117 suitably connected to the processor/controller(s) 114 for bidirectionally communicating with the intra-cranial module 135. Optionally, the telemetry unit 117 may also bidirectionally communicate with the portable communication device(s) 100 (such as, for example, with the mobile phone 70 and/or the laptop 9 and/or the AR headset 11). The extra-cranial module 141 and the intra-cranial module 135 (and optionally, the portable communication device(s) 100) may telemetrically exchange data, control signals and status signals there between.

The intra-cranial module 135 may include an intra-cranially implanted electronic circuitry module 152, two Ecog electrode arrays 144 and 146 suitably electrically connected to the electronic circuitry module 152 and an intra-cranial induction coil 146 (that may be similar to the induction coil 16 of FIG. 1) suitably electrically coupled to the electronic circuitry module 152 to provide electrical power to the electronic circuitry module 152 as is disclosed in more detail hereinabove. The Ecog array 142 may be disposed on the left DLPFC and the Ecog array 144 may be disposed on the right DLPFC as illustrated in FIG. 12. The cortical hemispheres are not shown in detail in FIG. 12, for the sake of clarity of illustration).

Turning to FIG. 13, the electronic circuitry module 152 includes one or more processor/controller(s) 124, a power conditioning and storage unit 177, electrically coupled to the intra-cranial induction coil 146, a telemetry unit 17 suitably electrically coupled to the processor/controller(s) 124, a memory/data storage unit 16 suitably electrically connected to the processor/controller(s) 124 and a signal conditioning and digitizing unit(s) 126 electrically connected to the Ecog arrays 142 and 144 to receive sensed signals from the electrodes of the Ecog arrays 142 and 144. The conditioning and digitizing unit(s) 126 is also connected to the processor/controller(s) 126 for providing digitized sensed Ecog signal's data to the processor/controller(s) 126.

The telemetry unit 17 may communicate bidirectionally with the telemetry unit 117 of the extra-cranial module 141, enabling bidirectional wireless transfer of data, control signals and status signals between the processor/controller 114 and the processor controller(s) 124.

It is noted that the power conditioning and storage unit 177 may include suitable circuitry (not shown in detail in FIG. 12 for conditioning electrical currents induced in the intra-cranial induction coil 146 by an extra-cranially placed second induction coil (not shown in FIGS. 12-13, for the sake of clarity of illustration, but see the induction coil 19 of FIG. 1 for an example) that may be placed on the scalp of the head 4 of the patient. Alternating currents passing within such an extra-cranially placed second induction coil induce alternating currents within the intra-cranial first induction coil. The alternating currents flowing within the intra-cranial induction coil 146 may be rectified by suitable current rectifying diode bridge circuitry (not shown) included in the power conditioning and storage unit 177 and may be stored by any suitable charge storage device (not shown) such as, for example, a super-capacitor, a capacitor, or a rechargeable electrochemical cell included within the power conditioning and storage unit 177.

The power conditioning and storage unit 177 is used for energizing any of the current requiring electrical components of the electronic circuitry module 152. It is noted that the electrical connections supplying electrical power to the components of the electronic circuitry module 152 are not shown in FIGS. 12-13 for the sake of clarity of illustration.

In operation, the system 140 may use any of the methods disclosed in the present application for delivering therapeutic stimulation for treating a mood disorder. For example, the Ecog arrays 142 and 144 may sense signals from the left DLPFC and/or the right DLPFC, respectively, the sensed signals may be conditioned (amplified and/or filtered) and digitized by the signal conditioning and digitizing unit(s) 126 and fed to the processor/controller(s) 124 for processing (according to any of the processing methods disclosed in the present application. If the processor/controller(s) 124 the system 140 detects that the patient is depressed. The system 140 may use the extra-cranial module 141 to stimulate one or more deep brain structures by using the NTIS method as disclosed hereinabove using the electrodes 145A, 145B, 147A and 147B and the stimulus generator 118. Any of the deep brain structure(s) disclosed in the present application may then be stimulated using the extra-cranial module 141 to treat a depressive mood of patient. Alternatively and/or additionally, any of the Ecog arrays 142 and 144 may be used by the system 140 to deliver cortical stimulation to the left DLPFC and/or to the right DLPFC, respectively, and/or to both the left DLPFC and the right DLPFC.

Having a sensing/stimulating device for sensing/stimulating the left DLPFC (such as, for example the Ecog array 142) and another sensing/stimulating device for sensing/stimulating the right DLPFC (such as, for example the Ecog array 144) may allow simultaneous machine learning optimized sTMS-like frequencies of stimulation delivered to the right DLPFC and rTMS-like frequencies of stimulation delivered to the left DLPFC which may both have independent efficacy for treating depression. It is noted that the systems disclosed herein are not limited to using intra-cranially implanted ECOG arrays for sensing and stimulating in the left and/or right DLPFC, but other types of more or less invasive stimulation/sensing devices may be used. For example two intra-calvarial implants (such as, but not limited to the implant 20 of FIG. 1) may be implanted in the calvarial bone overlying the left and the right DLPFC and may be used for sensing and stimulation of the left and right DLPFC, respectively. Other types of usable sensing/stimulating devices may include among others, mesh type injectable electronics, neural dust and stentrode type electrode arrays.

The methods for construction and for use of such diverse types of electrodes and electrode arrays and their associated electronic circuits, usable in the systems for treating a mood disorder of the present application, are described in detail, inter alia, in some of the following references:
1. Jeneva A. Cronin, Jing Wu, Kelly L. Collins, Devapratim Sarma, Rajesh P. N. Rao, Jeffrey G. Ojemann & Jared D. Olson. “Task-Specific Somatosensory Feedback via Cortical Stimulation in Humans.”, IEEE Transactions on Haptics, DRAFT. DOI: 10.1109/TOH.2016.2591952.
2. Kay Palopoli-Trojani, Virginia Woods, Chia-Han Chiang, Michael Trumpis & Jonathan Viventi. “In vitro Assessment of Long-Term Reliability of Low-Cost ECoG Arrays.”, Micro Electro Mechanical Systems, 2016, IEEE International Conference, 24-28 Jan. 2016, DOI: 10.1109/MEMSYS.2016.7421580.
3. Shota Yamagiwa, Makoto Ishida & Takeshi Kawano. “SELF-CURLING AND —STICKING FLEXIBLE SUBSTRATE FOR ECoG ELECTRODE ARRAY”, Micro Electro Mechanical Systems, 2013, IEEE₂₆ₕ International Conference, 20-24 Jan. 2013. DOI: 10.1109/MEMSYS.2013.647428.
4. Yusuke Morikawa, Shota Yamagiwa, Hirohito Sawahata, Makoto Ishida & Takeshi Kawano. “AN ORIGAMI-INSPIRED ULTRASTRETCHABLE BIOPROBE FILM DEVICE”, MEMS 2016, Shanghai, CHINA, 24-28 Jan. 2016, 978-1-5090-1973-1/16/$31.00 ©2016 IEEE, PP. 149-152.
5. Nikita Pak, Joshua H. Siegle, Justin P. Kinney, Daniel J. Denman, Tim Blanche & Ed S. Boyden. Closed-loop, ultraprecise, automated craniotomies. Journal of Neurophysiology 113, April 2015, Pp. 3943-3953.
6. Tian-Ming Fu, Guosong Hong, Tao Zhou, Thomas G Schuhmann, Robert D Viveros & Charles M Lieber., “Stable long-term chronic brain mapping at the single-neuron level.”, Nature Methods, Vol. 13, No. 10, October 2016, Pp. 875-882.
7. Chong Xie, Jia Liu, Tian-Ming Fu, Xiaochuan Dai, Wei Zhou & Charles M. Lieber., “Three-dimensional macroporous nanoelectronic networks as minimally invasive brain probes.”, Nature Materials, Vol. 14, December 2015, Pp. 1286-1292.
8. Guosong Hong, Tian-Ming Fu, Tao Zhou, Thomas G. Schuhmann, Jinlin Huang, & Charles M. Lieber. “Syringe Injectable Electronics: Precise Targeted Delivery with Quantitative Input/Output Connectivity”, Nano Letters, Vol. 15, August 2015, Pp. 6979-6984. DOI: 10.1021/acs.nanolett.5b02987.
9. Jia Liu, Tian-Ming Fu, Zengguang Cheng, Guosong Hong, Tao Zhou, Lihua Jin, Madhavi Duvvuri, Zhe Jiang, Peter Kruskal, Chong Xie, Zhigang Suo, Ying Fang & Charles M. Lieber. “Syringe-injectable electronics.”, Nature Nanotechnology, Vol. 10, July 2015, Pp. 629-636. DOI: 10.1038/NNANO.2015.115.
10. David T. Bundy, Mrinal Pahwa, Nicholas Szrama & Eric C. Leuthardt, Decoding three-dimensional reaching movements using electrocorticographic signals in humans”, Journal of Neural Engineering, Vol. 13, No. 2, 2016, Pp. 1-18. DOI:10.1088/1741-2560/13/2/026021.
11. Takufumi Yanagisawa, Masayuki Hirata, Youichi Saitoh, Haruhiko Kishima, Kojiro Matsushita, Tetsu Goto, Ryohei Fukuma, Hiroshi Yokoi, Yukiyasu Kamitani & Toshiki Yoshimine, “Electrocorticographic Control of a Prosthetic Arm in Paralyzed Patients.”, Annals of Neurology, Vol. 71, No. 3, March 2012, Pp. 353-361. DOI: 10.1002/ana.22613.
12. Wei Wang, Jennifer L. Collinger, Alan D. Degenhart, Elizabeth C. Tyler-Kabara, Andrew B. Schwartz, Daniel W. Moran, Douglas J. Weber, Brian Wodlinger, Ramana K. Vinjamuri, Robin C. Ashmore, John W. Kelly & Michael L. Boninger. “An Electrocorticographic Brain Interface in an Individual with Tetraplegia”, Plos One, Vol. 8, No. 2, February 2013, Pp. 1-8. DOI:10.1371/journal.pone.0055344.
13. Kay Palopoli-Trojani, Virginia Woods, Chia-Han Chiang, Michael Trumpis & Jonathan Viventi., “In vitro assessment of long-term reliability of low-cost ECoG arrays.”, Engineering in Medicine and Biology Society, 38th Annual International Conference of the IEEE, 16-20 Aug. 2016.
14. L. Muller, S. Felix, K. Shah, K. Lee, S. Pannu & E. Chang. “Thin-Film, Ultra High-Density Microelectrocorticographic Decoding of Speech Sounds in Human Superior Temporal Gyrus.”, Lawrence Livermore National Laboratory, IEEE Engineering in Medicine and Biology Conference, Orlanda, FL, United States, Aug. 16, 2016 through Aug. 20, 2016. LLNL-CONF-684084.
15. Jonathan Viventi, et al., “Flexible, Foldable, Actively Multiplexed, High-Density Electrode Array for Mapping Brain Activity in vivo.”, Nature Neuroscience, Vol. 14, No. 12, Pp. 1599-1605. DOI:10.1038/nn.2973.
16. Thomas J. Oxley et al. Minimally invasive endovascular stent-electrode array for high-fidelity, chronic recordings of cortical neural activity. Nature Biotechnology, Vol. 34, No. 3, February 2016. DOI:10.1038/nbt.3428.
17. Edward S. Boyden, Feng Zhang, Ernst Bamberg, Georg Nagel & Karl Deisseroth, “Millisecond-timescale, genetically targeted optical control of neural activity”, Nature Neuroscience, Vol. 8, No. 9, September 2005, Pp. 1263-1268. DOI:10.1038/nn1525.
18. Karl Deisseroth. “Optogenetics”, Nature Methods, Vol. 8, No. 1, January 2011, Pp. 26-29. DOI: 10.1038/NMETH.F.324.
19. Karl Deisseroth. “Optogenetics: 10 years of microbial opsins in neuroscience, “Nature Neuroscience, Vol. 18, No. 9, September 2015, Pp. 1213-1225.
20. Andre Berndt Karl Deisseroth.” Expanding the optogenetics toolkit: A naturally occurring channel for inhibitory optogenetics is discovered.” Science, Vol. 349, No. 6248, Aug. 7, 2015, Pp. 590-591.
21. S. Yamagiwa, M. Ishida & T. Kawano., “Flexible parylene-film optical waveguide arrays.”, Applied Physics Letters, Vol. 107, No. 083502, 2015, Pp. 1-5. DOI: 10.1063/1.4929402.
22. Michael Joshua Frank, Johan Samanta, Ahmed A. Moustafa & Scott J. Sherman. “Hold Your Horses: Impulsivity, Deep Brain Stimulation, and Medication in Parkinsonism.”, Science., Vol 318, No. 5854, December 2007, Pp. 1309-1312. DOI: 10.1126/science.1146157.
23. David J. Foster & Matthew A. Wilson. “Reverse replay of behavioural sequences in hippocampal place cells during the awake state.”, Nature 04587, Pp. 1-4. DOI:10.1038.
24. Nir Grossman, David Bono, Nina Dedic, Suhasa B. Kodandaramalah, Andrii Rudenko, Ho-Jun Suk, Antonino M. Cassara, Esra Neufeld, Niels, Li Huei Tsai, Alvaro Pascual-Leone and Edwards S. Boyden, “Non-Invasive Deep Brain Stimulation via Temporally Interfering Electric Fields”, Cell 169, pp 1029-1041, Jun. 1, 2017.
25. U.S. Pat. No. 8,121,694 to Molnar et al. entitled “Therapy control based on a patient movement state”.

While the system 140 uses NTIS for non-invasively stimulating one or more deep brain structures and one or more invasive electrode sets, such as, for example the Ecog electrode arrays 142 and 144 (or other types of electrode arrays such as, for example UTAH electrode arrays with electrodes that may penetrate the surface of the cortex), this exemplary configuration is not obligatory to practice the methods disclosed herein. While the non-invasiveness of the stimulating electrodes in NTIS simplifies the stimulation procedure, the user has to be tethered to the extra-cranial module 141 (in cases where the module 141 is a large static module) or may have to carry (or wear the module 141 in cases in which the module 141 is implemented as a small lightweight module that can be carried by the user). Additionally, using extra-cranial electrodes to perform NTIS may be inconvenient to the user, may be visibly unaesthetic and may also require frequent maintenance and care to avoid inadvertent electrode movements or undesirable variations in the electrical coupling characteristics of such extra-cranial stimulating electrodes to the skin.

Turning to FIGS. 14-15, all of the components of the system 160 are intra-cranially disposed except for the portable communication device unit(s) 100 (such as, for example, the mobile phone 70 and/or the laptop 9 and/or the AR headset 11) which is disposed outside the patient and some or all of the auxiliary sensor(s) 15 which may be attached to the patient or implanted in the body of the patient or worn by the patient, as disclosed in detail herein above. The portable communication device(s) 100 may be wirelessly connected to the cloud 31 and may exchange data and/or control signal/commands with a remote processor (not shown in FIG. 13) in the cloud 31, as disclosed in detail hereinabove with respect to the system 10 of FIG. 1.

The system 160 may include an intra-cranially implanted electronics module 162, three intra-cranially implanted Ecog electrode arrays 164, 166 and 168 electrically connected to the electronics module 162, and an intra-cranial induction coil 146 electrically connected to the electronics module 162. The Ecog electrode array 168 may be disposed on the DLPFC or on a part or portion of the PFC. In accordance with some embodiments of the system 160, the Ecog electrode array 168 may be disposed on the PFC regions of both cortical hemispheres as illustrated in FIG. 14 enabling selective sensing and/or selective stimulation of either the left DLPFC and/or The right DLPFC by suitable selection of individual electrodes 168A of the Ecog electrode array 168 for sensing and/or for stimulation.

Alternatively, in accordance with some embodiments of the system 160, the Ecog electrode array 168 may be disposed on the PFC or part thereof in the right cortical hemisphere (for sensing and/or stimulation of the right DLPFC). Alternatively, in accordance with other embodiments of the system 160, the Ecog electrode array 168 may be disposed on the PFC or part thereof in the left cortical hemisphere (for sensing and/or stimulation of the left DLPFC).

In some embodiments, the Ecog electrode array 164 may be disposed on the left cortical hemisphere or on a part of the left cortical hemisphere and the Ecog electrode array 166 may be disposed on the right cortical hemisphere or on a part of the right cortical hemisphere.

Turning now to FIG. 15, the system 160 may include one or more processor/controller(s) 14, a memory/data storage 16 suitably connected to the processor/controller(s) 14, a telemetry unit 17 suitably connected to the processor/controller(s) 14 for wirelessly transmitting data and/or control signals to the portable communication device(s) 100 (disposed outside the body of the patient). The system 160 may also include a power conditioning and storage unit 177 that is suitably electrically connected to the induction coil 146 to receive alternating currents therefrom (as disclosed in detail with respect to the induction coil 16 of FIG. 1). The structure and operation of the power conditioning and storage unit 177 is as disclosed hereinabove in detail with respect to the power conditioning and storage unit 177 of FIG. 13.

The system 160 may also include a stimulus generating module 170, suitably connected to and controlled by the processor/controller(s) 14. The stimulus generating module 170 includes a direct cortical stimulus generator 172 and a Frequency Interference Stimulus Generator 174 suitable for providing the different frequencies required for stimulation of deep brain structures.

The system 160 may also include one or more Multiplexing units 176. The multiplexing unit(s) 176 is/are suitably connected to the stimulus generator module 170 and to the processor/controller(s) 14 for controlling the delivery of stimuli from the frequency stimulus generator 174 to deep brain structures and to control the delivery of direct cortical stimulation from the cortical stimulus generator 172 to selected electrodes of the Ecog electrode arrays 164, 166 and 168.

The system 160 may also include one or more sensed signal conditioning and digitizing units 126 suitably electrically connected to the Ecog sensor arrays 164, 166 and 168 for conditioning the signals received from the electrodes included in the Ecog Arrays 164, 166 and 168 as disclosed in detail hereinabove with respect to FIG. 13.

The power conditioning and storage unit 177 may provide power for the operation of the electronics module 162. However, the connections providing power to the various components of the electronics module 162 are not shown in detail in FIG. 15 for the sake of clarity of illustration.

The portable communication devices(s) 100 may be any suitable communication device(s) capable of telemetrically communicating with the Telemetry unit 17 of the electronics module 162 (such as, for example the mobile phone 70 and/or the laptop 9 and/or the AR headset 11 of FIG. 14) or any other hand held or portable device including processing and controlling and wireless communication components as disclosed in detail hereinabove with respect to the system 10 of FIG. 1).

In operation, the system 160 may sense electrical signals from one or more cortical regions of the user by using one or more of the Ecog electrode arrays 164, 166 and 168 (such as, for example, sensing in the left DLPFC and/or the right DLPFC of the patient the electrode array 168). The sensed signals may be then conditioned (such as, for example, by being optionally filtered and amplified and) and then digitized by the sensed signals conditioning and digitizing unit(s) 126 and fed to the processor/controller(s) 14 for processing (according to any of the processing methods disclosed in the present application). If the processor/controller(s) 14 detects a depressed mood based on the processing of the sensed signals and on the EMA data, and (optionally) on the data from the auxiliary sensor(s) 15, the processor/controller(s) 14 may control the stimulus generator module 170 to stimulate one or more deep brain structures as follows. The processor/controller unit(s) 14 may control the multiplexing unit(s) 176 to select two spaced apart electrodes 164A and 164B of the Ecog electrode array 164 and two spaced apart electrodes 166A and i66B from the Ecog electrode array 166. After the electrodes have been selected, the processor/controller (s) 14 controls the frequency interference stimulus generator 174 to apply an oscillating current or voltage having an oscillation frequency f between the electrode pair 164A and 164B and to simultaneously apply an oscillating current or voltage signal having an oscillation frequency of f+Δf. The two frequencies f and f+Δf may be larger or equal than 1 KHz.

This temporal interference method of stimulation is somewhat similar but not identical to the NTIS method of Grossman et al., as described hereinabove but differs from the NTIS method is certain aspects. A first difference between the two methods is that while NTIS uses extra-cranial non-invasive stimulating electrodes to achieve non-invasive deep brain stimulation while the other method described herein (with respect to the system 160 uses intra-cranial stimulating electrodes (of intra-cranially implanted Ecog electrode arrays or other intra-cranial electrode arrays) for stimulating one or more deep brain structures. To clearly distinguish the method using intra-cranial stimulating electrodes disclosed herein from the NTIS method, we refer to the second method throughout the present application as intra-cranial temporal interference stimulation (ICTIS).

Another advantageous difference between NTIS and ICTIS is that while in NTIS the extra-cranial electrodes stay fixed at the same place on the head, the stimulating electrodes used may be changed very quickly by simply controlling the multiplexing unit(s) 176 to select different electrode pairs from any of the Ecog electrode arrays as the stimulating electrode pairs and deliver the two different interfering oscillation frequencies to any desired configuration of stimulating electrode pairs. This advantage may enable improved control and modulation of the size, shape and location of the neuronal recruiting focal region formed within the brain.

Furthermore, the configuration of the system 160 allows additional control of the stimulation because the stimulation electrodes may be varied almost instantly by passing the oscillating stimulation signals through any selected combination of spaced apart electrode groups by applying the stimulating oscillation with frequency f to a pair of two different electrode groups having any desired electrode number and electrode configuration of the Ecog electrode array 164 array and simultaneously applying the stimulating oscillation with frequency f+Δf. to another different pair of two different electrode groups having any desired electrode number and electrode configuration selected from the Ecog electrode array 166. This electrode grouping variation method within each pair of stimulating electrode may allow much finer control of the parameters of the neuronal recruiting envelope region in comparison to the NTIS method which features static fixed sized stimulation electrode pairs.

Moreover, another advantage of the ICTIS method is that the configuration and positions of the electrode group pairs or of the pairs of single electrodes may be rapidly alternated between differently positioned stimulating group pairs or between differently positioned single electrode pairs allowing rapid alternating changing of the position and/or size and/or shape of the neuronal recruiting region, that may result is alternating stimulation of differently positioned deep brain structures within the brain of the user. This variation may also be useful for achieving finer temporal control of the deep brain structure if necessary (this means that it may be possible to stimulate different deep brain structures at different times following the detection of the indication disclosed hereinabove.

Another feature of the system 160 is that it may allow not only the stimulation of deep brain structures by NTIS or by ICTIS but may also allow the stimulation of selected regions of some cortical regions by directly applying stimulating signals (such as, for example, pulses or stimulating pulse trains) to any selected electrodes (or electrode pairs, or electrode groups). For example, the processor/controller(s) 14 may control the multiplexing unit(s) 176 and the direct cortical stimulus generator 172 to deliver direct stimuli to any desired cortical regions underlying the Ecog electrode arrays 164 and 166, and/or to the DLPFC or any part thereof through the electrodes of the Ecog electrode array 168, or to any selected combinations of the right DLPFC, the left DLPFC and other cortical regions underlying the Ecog electrode arrays 164 and 166.

Furthermore, by using suitable multiplexing control, it may be possible to perform several types of stimulation regimes including, for example, simultaneous stimulation of one or more deep brain structures and one or more cortical regions (such as, for example the left and the right DLPFC), simultaneous stimulation of one or more different cortical regions only (for example, the right DLPFC and left DLPFC), stimulation of a single deep brain structure (by ICTIS), stimulation of a single cortical region or a part thereof by direct stimulation through a selected one of the Ecog electrode arrays 164, 166 and 168. Any combinations and permutation of such stimulation regimes/methods may be performed.

Another advantage of using ICTIS over NTIS for stimulating of any selected combination of deep brain structure stimulation and direct stimulation of one or more cortical regions is that while in NTIS in which the electrodes are coupled to the scalp with an electrically conducting gel or paste, it may be very difficult to keep the stimulating electrodes at exactly the same position on the scalp for extended periods of time due to accidental sliding or dislodgment of the stimulating electrodes, the use of intra-cranially implanted electrode array (like Ecog arrays or other intra-cranial arrays) this problem may be at least partially alleviated due to the internal positioning of the intra-cranial electrode arrays. Additionally, NTIS problems involving undesirable changes in scalp electrodes impedance due to drying of the coupling gel or paste used to electrically couple the stimulating electrodes to the patient's scalp may be solved by the intra-cranial placement of the Ecog arrays used in ICTIS.

It is further noted that in some embodiments of the systems of the present application, the intra-cranial electrode arrays (such as, for example, the Ecog arrays 144, 142, 164, 166, and 168) may be replaced with suitable intra-calvarial (IC) implants which are semi invasively implanted inside the calvarial bone without breaching or fully penetrating the inner table 6 of the calvarial bone 13. The advantages of using such IC implants may include reduced risk of complications to the patient, a much simpler and less costly implantation procedure that may possibly be performed in an outpatient day clinic without requiring hospitalization and less trauma to the patents. Such IC implants used for deep brain structure stimulation in ICTIS or for cortical region sensing/stimulation as disclosed in detail hereinabove for the IC implant 20 may advantageously result in increased electrode stability due to the anchoring of the IC implants to the outer table 5 of the cranial bone 13 (as may be seen for the IC implant 20 in FIG. 5), reducing the mass of tissue underlying the stimulating electrodes of the IC implant (as compared to the scalp electrodes used in NTIS) to reduce the required stimulating currents and greatly simplifying and shortening the implantation procedure to reduce patient's inconvenience and reduce or eliminate hospitalization time.

The IC implants usable in the systems of the present application may be similar to the IC implant 20 configured for sensing and stimulating cortical regions but may also be different IC implants specifically configured for delivering deep brain structure stimulation and/or sensing/stimulation of cortical regions.

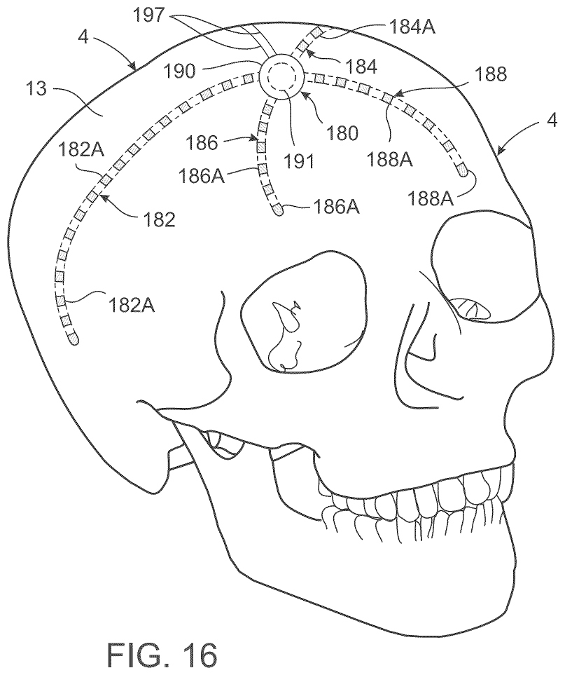

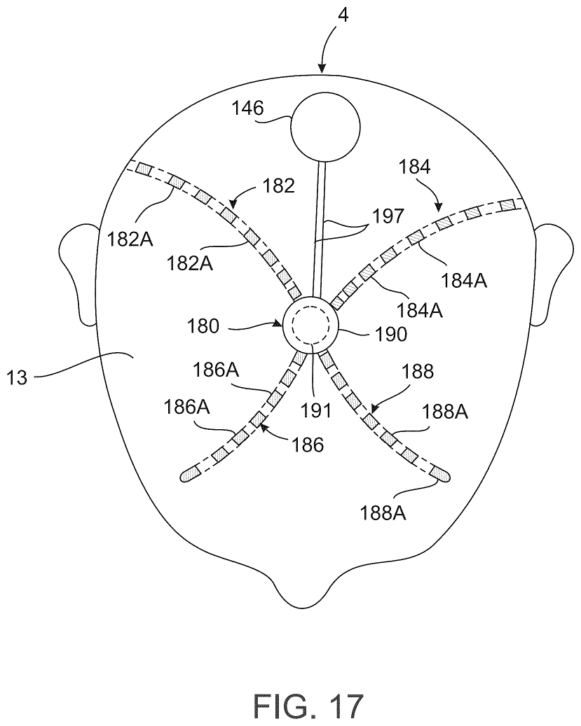

Reference is now made to FIGS. 16-17. FIG. 16 is a schematic isometric view diagram illustrating a human skull with an implanted intra-calvarial implant suitable for delivering deep brain structure stimulation to a patient's brain implanted in the calvarial bone of the skull in accordance with an embodiment of the intra-calvarial implants of the present application. FIG. 17 is a top view of the skull illustrated in FIG. 16.

It is noted that FIGS. 16-17. Do not show other components of the system that may use the illustrated the IC implant 180 and are provided to indicate the position of the IC implant 180 and its components in the calvarial bone of the skull. Such system components may include the portable communication device(s) 100, the effector device(s) 14 and the auxiliary sensor(s) 15 as disclosed for the system 10 of FIG. 1.

The IC implant 180 may include a housing 190 similar to the housing 202 and four elongated flexible intra-calvarial electrode arrays 182, 184, 186 and 188. The intra-calvarial electrode array 182 has multiple electrically conducting electrodes 182A there along. The intra-calvarial electrode array 184 has multiple electrically conducting electrodes 184A there along. The intra-calvarial electrode array 186 has multiple electrically conducting electrodes 186A there along. The intra-calvarial electrode array 188 has multiple electrically conducting electrodes 188A arranged there along. The housing 190 may be made from materials similar to those disclosed for the housing 202 of the implant 200 hereinabove.

When the IC implant 180 is implanted, a hole or opening may be drilled in the outer table 5 and the cancellous bone (diplöe) 7 of the calvarial bone 13, for accepting therein the housing 190. Four elongated passages (not shown) may then be drilled or laser ablated within the cancellous bone layer 7 in a direction roughly parallel to the plane of the inner table 6 for accepting therein the four flexible elongated electrode arrays 182, 184, 186 and 188. Preferably, the passages are made close to or bordering the external surface 6B of the inner table 6. The flexible electrode arrays182, 184, 186 and 188 may then be inserted into the four passages, and the housing 190 may then be inserted into the opening drilled in the upper table 5 such that it is flush with the outer surface 5A of the outer table 5 (see FIG. 5) and sealed and attached to the outer table 5 with a biocompatible sealant or glue, as disclosed in detail for the implant 20.

The IC implant 180 may also include a miniaturized electronic module 191 illustrated in dashed lines to indicate that it is disposed within the housing 190. The electronic module 191 may include all the components of the extra-cranial module 141 of FIG. 12 except that all components of the electronic module are miniaturized to fit within the housing 190, and except that the electronic module may also include the multiplexing unit(s) 176 (of FIG. 15) connected between the processor/controller(s) 114 of the electronic module 191 and all the electrodes 182A, 184A, 186A and 188A of the elongated electrode arrays 182, 184, 186, and 188, respectively.

The multiplexing unit(s) 176 may allow connecting any selected pairs of the electrodes 182A, 184A, 186A and 188A to the stimulus generator 118 of the electronic module 191 for delivering ICTIS stimulation to any selected region of the brain including deep brain structures and/or cortical regions. Optionally, in some embodiments, the electronic module 191 may also include the signal conditioning and digitizing unit(s) 126 of the electronics module 152 (FIG. 13) which may be suitably connected to the multiplexing units 176 and the processor/controller 114 to enable sensing cortical signals from selected electrodes of the elongated electrode arrays 182, 184, 186 and 188.

The electronic module 191 of the implant 180 may be suitably connected to an induction coil 146 by suitable isolated electrically conducting wires 197, as disclosed in detail hereinabove for receiving power from another induction coil positioned on the scalp (the scalp is not shown for the sake of clarity of illustration).

The elongated electrode arrays 182, 184, 186 and 188 are suitably sealingly attached to the housing 191 and include multiple isolated wires (not shown in FIGS. 16-17 for the sake of clarity of illustration) that allow “addressing” each of the electrodes to by the multiplexing unit(s) 176 of the electronic module 191.

Stimulation of deep brain structures may be performed by the electronic module 191 by the same frequency interference methods disclosed hereinabove with respect to the systems 140 and 160 hereinabove. The selection of differently positioned specific electrode pairs for delivering the stimuli at frequency f and f+Δf may allow fine tuning of the stimulation of deep brain structures if necessary and may allow greater flexibility in stimulating both selected deep brain structures and the more superficial cortical regions (such as, for example, the right DLPFC and the left DLPFC). Thus, the use of the IC implant 180 may allow both sensing of cortical regions and stimulation of deep brain structures and/or cortical regions by interlacing sensing and stimulation time periods.

It is noted that while the methods and systems disclosed hereinabove may specifically stimulate the left and/or the right DLPFC regions (which may or may not be combined with stimulation of one or more deep brain structure), in some embodiments of the methods and systems a different cortical stimulation target may be used. For example other regions of the prefrontal cortex (PFC) may be the cortical stimulation target. Such stimulation of other PFC regions may or may not be combined with stimulation of deep brain structures. Evidence for the efficacy of sTMS may be found in the article by Klein et al. (1999) cited in the reference list hereinbelow.

In is noted that while in the systems disclosed herein the portable communication device(s) 100 are illustrated as including the mobile phone 70, the laptop 9 and the AR headset 11, this is not obligatory to practicing the invention and the communication device(s) 100 may include any suitable type of portable communicating device(s) such as a smartphone, a tablet, a phablet, a notebook, a laptop, a mobile computer, an AR headset having communication capabilities, or any other similar type of portable device having processing capabilities, communication capabilities and means of displaying content to the patient. In addition, if the patient has a mobile phone or smartphone for providing the EMA input and patient self assessment data, the laptop 9 may be substituted by a non-portable computer such as, for example, a desktop computer, a workstation, or a remote server or remote personal computer for providing the caretaker with logged patient data and/or warning signals and/or patient status information.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

All publications, patents and patent applications mentioned in this specification (including the references of the list appended hereinafter) are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

### REFERENCES

Albert, G. C., Cook, C. M., Prato, F. S., and Thomas, A. W. (2009). Deep brain stimulation, vagal nerve stimulation and transcranial magnetic stimulation: an overview of stimulation parameters and neurotransmitter release. Neurosci. Biobehav. Rev. 33, 1042-1060.
Alesci, S., Martinez, P. E., Kelkar. S., Ilias, I., Ronsaville, D. S., Listwak, S. J., Ayala, A. R., Licinio, J., Gold, H. K., Kling, M. A., Chrousos, G. P., and Gold, P. W. (2005). Major depression is associated with significant diurnal elevations in plasma interleukin-6 levels, a shift of its circadian rhythm. and loss of physiological complexity in its secretion: clinical implications. J. Clin. Endocrinol. Metab. 90, 2522-2530.
Allain, C. C., Poon, L. S., Chan, C. S. C., Richmond, W., and Fu, P. C. (1974). Enzymatic determination of total serum cholesterol. Clin. Chem. 20, 470-475.
Avery, D. H., Holtzheimer Il1. P. E., Fawaz, W., Russo, J., Neumaier, J., Dunner, D. L., Haynor, D. R., Claypoole, K. H., Wajdik, C., and Roy-Byrne, P. (2006). A controlled study of repetitive transcranial magnetic stimulation in medication-resistant major depression. Biol. Psychiatry 59, 187-194.
Ballenger, J. C., and Post, R. M. (1980). Carbamazepine in manic-depressive illness: a new treatment. Am. J. Psychiatry 137, 782-790.
Barker, A. T., Jalinous, R., and Freeston, I. L. (1985). Non-invasive magnetic stimulation of human motor cortex. Lancet 325, 1106-1107.
Behrend, C. E., Cassim, S. M., Pallone, M. J., Daubenspeck, J. A., Hartov, A., Roberts, D. W., and Leiter, J. C. (2009). Toward feedback controlled deep brain stimulation: dynamics of glutamate release in the subthalamic nucleus in rats. J. Neurosci. Methods 180, 278-289.
Bejjani, B. P., Damier, P., Arnulf. I., Thivard. L., Bonnet, A. M., Dormont. D., Cornu, P., Pidoux, B., Samson, Y., and Agid, Y. (1999). Transient acute depression induced by high-frequency deep-brain stimulation. N. Engl. J. Med 340, 1476-1480.
Belmaker, R. H., and Agam, G. (2008). Major depressive disorder. N. Engl. J. Med. 358, 55-68.
Benabid, A. L., Pollak, P., Louveau, A., Henry, S., and de Rougemont, J. (1987). Combined (thalamotomy and stimulation) stereotactic surgery of the VIM thalamic nucleus for bilateral Parkinson disease. Appl. Neurophysiol. 50, 344-346.
Ben-Menachem, E., Hamberger, A., Hedner. T., Hammond. E. J., Uthman. J. S., Treig. T., Stefan, H., Ramsay, R. E., Wernicke, J. F., and Wilder, B. J.(1995). Effects of vagus nerve stimulation on amino acids and other metabolites in the CSF of patients with partial seizures. Epilepsy Res. 20. 221-227.
Ben-Menachem, E., Manon-Espaillat, R., Ristanovic, R., Wilder, B. J., Stefan, H., Mirza, W., Tarver, W. B., and Wemicke, J. F. (1994). Vagus nerve stimulation for treatment of partial seizures: 1. A controlled study of effects on seizures. First Intemational Vagus Nerve Stimulation Study Group. Epilepsia 35, 616-626.
Bhagwagar, Z., Rabiner, E. A., Sargent, P. A., Grasby, P. M., and Cowen, P. J. (2004). Persistent reduction in brain serotonin 1A receptor binding in recovered depressed men measured by positron emission tomography with [11C] WAY-100635. Mol. Psychiatry 9.386-392.
Bradley, R. G., Binder, E. B., Epstein, M. P., Tang, Y., Nair, H. P., Liu, W., Gillespie, C. F., Berg, T., Evces, M., Newport, D. J., Stowe, Z. N., Heim, C. M., Nemeroff, C. B., Schwartz, A., Cubells, J. F., and Ressler, K. J. (2008). Influence of child abuse on adult depression: moderation by the corticotropin-releasing hormone receptor gene. Arch. Gen. Psychiatry 65, 190-200.
Brody, A. L., Saxena, S., Stoessel, P., Gillies, A., Fairbanks, L. A., Alborzian, S., Phelps. M. E., Huang, S. C., Wu, H. M., Ho, M. L., Ho, M. K., Au, S. C., Maidment, K., and Baxter, L. R. (2001). Regional brain metabolic changes in patients with major depression treated with either paroxetine or interpersonal therapy. Arch. Gen. Psychiatry 58, 631-640.
Burke, H. M., Davis, M. C., Otte. C., and Mohr, D. C. (2005). Depression and cortisol responses to psychological stress: a meta-analysis. Psychoneuroendocrinology 30, 846-856.
Carpenter. L. L., Moreno. F. A., Kling, A., Anderson. G. M., Regenold, W. T., Labiner, D. M., and Price, L. H. (2004). Effect of vagus nerve stimulation on cerebrospinal fluid monoamine metabolites, norepinephrine, and gamma-aminobutyric acid concentrations in depressed patients. Biol. Psychiatry 56, 418-426.
Carroll, B. J., Cassidy, F., Naftolowitz, D., Tatham, N. E., Wilson, W. H., Iranmanesh, A., Liu, P. Y., and Veldhuis, J. D. (2007). Pathophysiology of hypercortisolism in depression. Acta Psychiatr. Scand. 115, 90-103.
Caspi, A., Sugden, K., Moffi T. E., Taylor. A., Craig, I. W., Harrington, H., McClay, J., Mill. J., Martin, J., Braithwaite, A., and Poulton, R. (2003). Influence of life stress on depression: moderation by a polymorphism in the 5-HTT gene. Science 301, 386-389.
Cepoiu, M., McCusker, J., Cole, M. G., Sewitch, M., Belzile, E., and Ciampi, A. (2008). Recognition of depression by non-psychiatric physicians—a systematic literature review and meta analysis. J. Gen. Intern. Med 23, 25-36.
Cohen, D., and Cuffin, B. N. (1991). Developing a more focal magnetic stimulator. Part I: some basic principles. Clin. Neurophysiol. 8, 102-111.
Coppen, A. (1967). The biochemistry of affective disorders. Br. J. Psychiatry 113, 1237-1264.
Coyne, J. C., Fechner-Bates, S., and Schwenk, T. L. (1994). Prevalence, nature, and comorbidity of depressive disorders in primary care. Gen. Hosp. Psychiatry 16, 267.
Cyberonics (2007). About Treatment Resistant Depression. VNS Therapy. Cyberonics. <www(dot)vnstherapy(dot)com/depression/vnsrightforme/abouttrd9(dot)asp>
Dantzer, R., O'Connor, J. C., Freund, G. G., Johnson, R. W., and Kelley, K. W. (2008). From inflammation to sickness and depression: when the immune system subjugates the brain. Nat. Rev. Neurosci. 9, 46-56.
Davidson, R. J., Pizzagalli. D., Nitschke, J. B., and Putnam, K. (2002). Depression: perspectives from affective neuroscience. Annu. Rev. Psychol. 53, 545-574.
Depression Guideline Panel (1994). Depression in primary care: detection, diagnosis, and treatment. J. Am. Acad. Nurse Pract. 6, 224-238.
Duman, R. S., Heninger, G. R., and Nestler, E. J. (1997). A molecular and cellular theory of depression. Arch. Gen. Psychiatry 54, 597.
Duman, R. S., Malberg, J., Nakagawa, S., and D'Sa, C. (2000). Neuronal plasticity and survival in mood disorders. Biol. Psychiatry 48, 732-739.
Duman, R. S., and Monteggia, L. M. (2006). A neurotrophic model for stress-related mood disorders. Biol. Psychiatry 59, 1116-1127.
Dumitriu, D., Collins, K., Alterman. R., and Mathew. S. J. (2008). Neurostimulatory therapeutics in management of treatment-resistant depression with focus on deep brain stimulation. Mt. Sinai J. Med. 75, 263-275.
Ellis, P. M., and Salmond, C. (1994). Is platelet imipramine binding reduced in depression?A meta-analysis. Biol. Psychiatry 36, 292-299.
Fava, M. (2003). Diagnosis and definition of treatment-resistant depression. Biol. Psychiatry 53, 649-659.
Fava. M., Borus, J. S., Alpert, J. E., Nierenberg. A. A., Rosenbaum. J. F., and Bottiglieri, T. (1997). Folate, vitamin B12, and homocysteine in major depressive disorder. Am. J. Psychiatry 154, 426-428.
Feng, X. J., Greenwald, B., Rabitz, H., Shea-Brown, E., and Kosut, R. (2007). Toward closed-loop optimization of deep brain stimulation for Parkinson's disease: concepts and lessons from a computational model. J. Neuroeng. Rehabil. 4, L14-L21.
Figiel, G. S., Epstein, C. M., McDonald, W. M., Amazon-Lece, J., Figiel, L., Saldivia, A., and Glover, S. (1998). The use of rapid-rate transcranial magnetic stimulation (rTMS) in refractory depressed patients. J. Neuropsychiatry Clin. Neurosci. 10, 20-25.
First, M. B., and Ross, R. (eds) (2000). American Psychiatric Association: Diagnostic and Statistical Manual of Mental Disorders, Text Revision. Washington, DC: R. R. Donnelly and Sons Company.
Fitzgerald, P. B., Benitez, J., de Castella, A., Daskalakis, Z. J., Brown, T. L., and Kulkarni. J. (2006). A randomized, controlled trial of sequential bilateral repetitive transcranial magnetic stimulation for treatment-resistant depression. Am. J. Psychiatry 163, 88-94.
Fitzgerald, P. B., Brown, T. L., and Daskalakis, Z. J. (2002). The application of transcranial magnetic stimulation in psychiatry and neurosciences research. Acta Psychiatr. Scand. 105, 324-340.
Fontaine, D., Mattei, V., Borg, M., von Langsdorff, D., Magnie, M. N., Chanalet, S., Robert, P., and Paquis, P. (2004). Effect of subthalamic nucleus stimulation on obsessive-compulsive disorder in a patient with Parkinson disease. J. Neurosurg. 100, 1084-1086.
Garrett, A., Lithgow, B. J., Gurvich, C., and Fitzgerald, P. (2008). EVestG™: Responses in Depressed Patients. 30th Annual International IEEE EMBS Conference, Vancouver. BC.
George, M. S., Rush, A. J., Marangell, L. B., Sackeim, H. A., Brannan, S. K., Davis, S. M., Howland, R., Kling, M. A., Moreno. F., Rittberg, B., Dunner, D., Schwartz. T., Carpenter, L., Burke, M., Ninan, P., and Goodnick, P. (2005). A one-year comparison of vagus nerve stimulation with treatment as usual for treatment-resistant depression. Biol. Psychiatry 58, 364-373.
George, M. S., Sackeim, H. A., Rush, A. J., Marangell, L. B., Nahas, Z., Husain, M. M., Lisanby, S. H, Burt, T., Goldman, J., and Ballenger. J. C. (2000). Vagus nerve stimulation: a new tool for brain research and therapy. Biol. Psychiatry 47, 287-295.
Goldapple, K., Segal, Z., Garson, C., Lau. M., Bieling, P., Kennedy. S., and Mayberg, H. (2004). Modulation of cortical-limbic pathways in major depression: treatment specific effects of cognitive behavior therapy. Arch. Gen. Psychiatry 61.34-41.
Golier, J. A., Marzuk, P. M., Leon, A. C., Weiner. C., and Tardiff, K. (1995). Low serum cholesterol level and attempted suicide. Am. J. Psychiatry 152, 419-423.
Goodman, W. K., and Insel, T. R. (2009). Deep brain stimulation in psychiatry: concentrating on the road ahead. Biol. Psychiatry 65, 263-266.
Greenberg. B. D., Malone, D. A., Friehs, G. M., Rezai, A. R., Kubu, C. S., Malloy. P. F., Salloway, S. P., Okun, M. S., Goodman, W. K., and Rassmussen, S. A. (2006). Three-year outcomes in deep brain stimulation for highly resistant obsessive-compulsive disorder. Neuropsychopharmacology 31, 2384-2393.
Grossman N, Bono D, Dedic N,. Kodandaramalah S. B., Rudenko A, Ho-Jun Suk H J., Cassara A. M., Neufeld E. N, Tsai L. H, Pascual-Leone A, and Boyden E. S., (2017) “Non-Invasive Deep Brain Stimulation via Temporally Interfering Electric Fields”, Cell 169, pp 1029-1041.
Halbig, T. D., Gruber, D., Kopp, U. A., Schneider, G. H., Trottenberg, T., and Kupsch, A. (2005). Pallidal stimulation in dystonia: effects on cognition, mood, and quality of life. J. Neurol. Neurosurg. Psychiatr. 76, 1713-1716.
Han, M., and McCreery, D. B. (2009). Microelectrode Technologies for Deep Brain Stimulation. Implantable Neural Prostheses 1. New York: Springer, 195-219.
Haidesty, D. E., and Sackeim, H. A. (2007). Deep brain stimulation in movement and psychiatric disorders. Biol. Psychiatry 61, 831-835.
Heils, A., Teufel, A., Petri. S., Stoeber, G., Riederer, P., Bengel, D., and Lesch, K. P. (1996). Allelic variation of human serotonin transporter gene expression. J. Neurochem. 66, 2621-2624.
Henry, T. R., Bakay, R. A. E., Votaw, J. R., Pennell. P. B., Epstein. C. M., Faber, T. L., Grafton. S. T., and Hoffman, J. M. (1998). Brain blood flow alterations induced by therapeutic vagus nerve stimulation in partial epilepsy: 1. Acute effects at high and low levels of stimulation. Epilepsia 39, 983-990.
Holsboer, F. (2000). The corticosteroid receptor hypothesis of depression. Neuropsychopharmacology 23, 477-500.
Holsboer, F., and Barden, N. (1996). Antidepressants and hypothalamic-pituitary-adrenocortical regulation. Endocr. Rev. 17, 187-205.
Jacobs, B. L., van Praag, H., and Gage, F. H. (2000). Adult brain neurogenesis and psychiatry: a novel theory of depression. Mol. Psychiatry 5, 262-269.
Janicak, P. G., O'Reardon, J. P., Sampson, S. M., Husain. M. M., Lisanby, S. H., Rado, J. T., Heart, K. L., and Demitrack, M. A. (2008). Transcranial magnetic stimulation in the treatment of major depressive disorder: a comprehensive summary of safety experience from acute exposure. extended exposure. and during rintroduction tratment. J. Clin. Psychiatry 69, 222-232.
Jimenez, F., Velasco, F., Salin-Pascual, R., Hemandez, J., Velasco, M., Criales, J. L., and Nicolini, H. (2005). A patient with a resistant major depression disorder trcated with deep brain stimulation in the inferior thalamic peduncle. Neurosurgery 57, 585-593.
Joost Asselbergs, Jeroen Ruwaard, Michal Ejdys, Niels Schrade, Marit Sijbrandij, and Heleen Riper, (2016) “Mobile Phone-Based Unobtrusive Ecological Momentary Assessment of Day-to-Day Mood: An Explorative Study”, Journal of Medical Internet Research, Vol 18 Issue 3 (DO: 10.2196/jmir.5505)
Judd, L. L., Akiskal, H. S., Maser, J. D., Zeller, P. J., Endicott, J., Coryell, W., Paulus, M. P., Kunovac, J. L., Leon. A. C., Mueller, T. I., Rice. J. A., and Keller, M. B. (1998). A prospective 12-year study of subsyndromal and syndromal depressive symptoms in unipolar major depressive disorders. Arch. Gen. Psychiatry 55, 694-700.
Karege, F., Perret, G., Bondolfi, G., Schwald, M., Bertschy, G., and Aubry J. M. (2002). Decreased serum brain derived neurotrophic factor levels in major depressed patients. Psychiatry Res. 109, 143-148.
Kearns, N. P., Cruickshank, C. A., McGuigan, K. J., Riley, S. A., Shaw, S. P., and Snaith, R. P. (1982). A comparison of depression rating scales. Br. J. Psychiatry 141, 45-49.
Kempermann, G., and Kronenberg, G. (2003). Depressed new neurons?—Adult hippocampal neurogenesis and a cellular plasticity hypothesis of major depression. Biol. Psychiatry 54, 499-503.
Kendler, K. S., Kuhn, J. W., Vittum, J., Prescott, C. A., and Riley, B. (2005). The interaction of stressful life events and a serotonin transporter polymorphism in the prediction of episodes of major depression: a replication. Arch. Gen. Psychiatry 62, 353-529.
Kessler, R. C., Berglund, P., Demler, O., Jin, R., Koretz, D., Merikangas, K. R., Rush, A. J., Walters. E. E., and Wang, P. S. (2003). The epidemiology of major depressive disorder: results from the National Comorbidity Survey Replication (NCS-R). JAMA 289, 3095-3105.
Kessler, R. C., Berglund, P., Demler, O., Jin. R., Merikangas, K. R., and Walters, E. E. (2005). Lifetime prevalence and age of onset distributions of DSM-IV disorders in the National Comorbidity Survey Replication. Arch. Gen. Psychiatry 62, 593-02.
Kirsch. I. (2002). The emperor's new drugs: an analysis of antidepressant medication data submitted to the U.S. Food and Drug Administration. Prev. Treat. 5, 1-11.
Kirsch, I., Deacon, B. J., Huedo-Medina, T. B., Scoboria, A., Moore, T. J., and Johnson, B. T. (2008). Initial severity and antidepressant benefits: a meta-analysis of data submitted to the Food and Drug Administration. PLoS Med 5, 0260-0268. doi: 10.1371/journal. pmed.0050045.
Klein, E., Kreinin, I., Chistyakov, A., Koren, D., Mecz, L., Marmur, S., Ben-Shachar, D., and Feinsod, M. (1999). Therapeutic efficacy of right prefrontal slow repetitive transcranial magnetic stimulation in major depression. Arch. Gen. Psychiatry 56, 315-320.
Konsman, J. P., Vigues, S., Mackerlova, L., Bristow. A., and Blomqvist, A. (2004). Rat brain vascular distribution of interleukin-1 type-1 receptor immunoreactivity: relationship to patterns of inducible cyclooxygenase expression by peripheral inflammatory stimuli. J. Comp. Neurol. 472, 113-129.
Kosel, M., Sturm, V., Frick, C., Lnartz, D., Zeidler, G., Brodesser, D., and Schlaepfer, T. E. (2007). Mood improvement after deep brain stimulation of the internal globus pallidus for tardive dyskinesia in a patient suffering from major depression. J. Psychiatr. Res. 41, 801-803.
Krahl, S. E., Clark, K. B., Smith, D. C., and Browning. R. A. (1998). Locus coeruleus lesions suppress the seizure-attenuating effects of vagus nerve stimulation. Epilepsia 39, 709-714.
Kroenke, K., Spitzer, R. L., and Williams. J. B. W. (2001). The PHQ-9: validity of a brief depression severity measure. J. Gen. Intern. Med. 16, 606-613.
Kunugi, H., Takei, N., Aoki, H., and Nanko, S. (1997). Low serum cholesterol in suicide attempters. Biol. Psychiatry 41, 196-200.
Lacasse, J. R., and Leo, J. (2005). Serotonin and depression: a disconnect between the advertisements and the scientific literature. PLoS Med. 2, 1211-1216. [doi: 10.1371/journal. pmed.0020392].
Lozano. A. M., Mayberg, H. S., Giacobbe, P., Hamani. C., Craddock, R. C., and Kennedy, S. H. (2008). Subcallosal cingulate gyrus deep brain stimulation for treatment-resistant depression. Biol. Psychiatry 64, 461-467.
Malone. D. A., Dougherty, D. D., Rezai, A. R., Carpenter, L. L., Friehs, G. M., Eskandar, E. N., Rauch, S. L., Rassmussen, S. A., Machado, A. G., Kubu, C. S., Tyrka, A. R., Price, L. H., Stypulkowski. P. H., Giftakis, J. E., Rise, M. T., Malloy, P. F., Salloway, S. P., and Greenberg. B. D. (2009). Deep brain stimulation of the ventral capsule/ventral striatum for treatment-resistant depression. Biol. Psychiatry 65, 267-275.
Manji, H. K., Drevets, W. C., and Chamey, D. S. (2001). The cellular neurobiology of depression. Nat. Med. 7, 541-547.
Mann. J. J. (2005). The medical manage-ment of depression. N. Engl. J. Med. 353, 1819. Marangell, L. B., Martinez, M., Jurdi, R. A, and Zboyan, H. (2007). Neurostimulation therapies in depression: a review of new modalities. Acta Psychiatr. Scand. 116, 174-181.
Marangell, L. B., Rush, A. J., George, M. S., Sackeim, H. A., Johnson, C. R., Husain, M. M., Nahas, Z., and Lisanby, S. H., (2002). Vagus nerve stimulation (VNS) for major depressive episodes: one year outcomes. Biol. Psychiatry 51, 280-287.
Martin, S. D., Martin, E., Rai, S. S., Richardson, M. A., and Royall, R. (2001). Brain blood flow changes in depressed patients treated with interpersonal psychotherapy or venlafaxine hydrochloride. Arch. Gen. Psychiatry 58, 641-648.
Mayberg, H. S. (1997). Limbic-cortical dysregulation: a proposed model of depression. J. Neuropsychiatry Clin. Neurosci. 9, 471-481.
Mayberg, H. S., Brannan, S. K., Tekell, J. L., Silva, J. A., Mahurin, R. K., McGinnis, S., and Jerabek, P. A. (2000). Regional metabolic effects of fluoxetine in major depression: serial changes and relationship to clinical response. Biol. Psychiatry 48, 830-843.
Mayberg, H. S., Liotti, M., Brannan, S. K., McGinnis, S., Mahurin, R. K., Jerabek, P. A., Silva, J. A., Tekell, J. L., Martin, C. C., Lancaster, J. L., and Fox, P. T. (1999). Reciprocal limbic-cortical function and negative mood: converging PETfindingsindepression and normal sadness. Am. J. Psychiatry 156, 675-682.
Mayberg, H. S., Lozano, A. M., Voon, V., McNeely, H. E., Seminowicz, D., Hamani, C., Schwalb, J. M., and Kennedy, S. H. (2005). Deep brain stimulation for treatment-resistant depression. Neuron 45, 651-660.
McCreery, D. B., Yuen, T. G. H., Agnew, W. F., and Bullara, L. A. (1997). A characterization of the effects of neuronal excitability due to prolonged micro-stimulation with chronically implanted microelectrodes. IEEE Trans. Bionmed Eng. 44, 931-939.
Merali. Z., Du. L., Hrdina, P., Palkovitz, M., Faludi, G., Poulter, M. O., and Anisman, H. (2004). Dysregulation in the suicide brain: mRNA expression of corticotropin-releasing hormone receptors and GABA(A) receptor subunits in frontal cortical brain region. J. Neurosci. 24, 1478-1485.
Milak, M. S., Parsey, R. V., Keilp, J., Oquendo, M. A., Malone, K. M., and Mann, J. J. (2005). Neuroanatomic correlates of psychopathologic components of major depressive disorder. Arch. Gen. Psychiatry 62, 397-408.
Mossner. R., Mikova, O., Koutsilieri, E., Saoud. M., Ehlis, A. C., Muller. N., Fallgatter, A. J., and Riederer, P. (2007). Consensus of the WFSBP Task Force on biological markers: biological markers in depression. World J. Biol. Psychiatry 8, 141-174.
Mueller. T. I., Leon, A. C., Keller, M. B., Solomon, D. A., Endicott, J., Coryell, W., Warshaw, M., and Maser, J. D. (1999). Recurrence after recovery from major depressive disorder during 15 years of observational follow-up. Am. J. Psychiatry 156, 1000-1006.
Nemeroff, C. B., Mayberg, H. S., Krahl, S. E., McNamara, J., Frazer, A., Henry, T. R., George, M. S., Charney, D. S., and Brannan, S. K. (2006). VNS therapy in treatment-resistant depression: clinical evidence and putative neurobiological mechanisms. Neuropsychopharmacology 31, 1345-1355.
Nemeroff, C. B., Widerlov, E., Bissette, G., Walleus, H., Karlsson, I., Eklund, K., Kilts, C. D., Loosen. P. T., and Vale. W. (1984). Elevated concentrations of CSF corticotropin-releasing factor-like immunoreactivity in depressed patients. Science 226, 1342-1344.
Nestler, E. J., Barrot, M., DiLeone, R. J., Eisch, A. J., Gold, S. J., and Monteggi, L. M. (2002). Neurobiology of depression. Neuron 34, 13-25.
Neuronetics (2009). About TMS Therapy. NeuroStar TMS Therapy, Retrieved Aug. 6, 2009, from www(dot)neurostartms(dot)com.
Nibuya, M., Morinobu, S., and Duman, R. S. (1995). Regulation of BDNF and trkB mRNA in rat brain by chronic electroconvulsive seizure and antidepressant drug treatments. J. Neurosci. 15, 7539-7547.
Nuttin, B., Cosyns, P., Demeulemeester, H., Gybels, J., and Meyerson, B. (1999). Electrical stimulation in anterior limbs of internal capsules in patients with obsessive-compulsive disorder. Lancet 354, 1526.
O'Brien, S. M., Scully, P., Fitzgerald, P., Scott, L. V., and Dinan, T. G. (2007). Plasma cytokine profile in depressed patients who fail to respond to selective serotonin reuptake inhibitor therapy. J. Psychiatr. Res. 41, 326-331.
Ongur, D., An, X., and Price, J. L. (1998). Prefrontal cortical projections to the hypothalamus in macaque monkeys. J. Comp. Neurol. 401, 480-505.
Pascual-Marqui, R. D., Michel, C. M., and Lehmann. D. (1994). Low resolution electromagnetic tomography: a new method for localizing electrical activity in the brain. Int. J. Psychophysiol. 18.49-65.
Peretti, S., Judge, R., and Hindmarch, I. (2000). Safety and tolerability considerations: tricyclic antidepressants vs. selective serotonin reuptake inhibitors. Acta Psychiatr. Scand. 101, 17-25.
Piallat, B., Chabardes, S., Devergnas, A., Torres, N., Allain, M., Barrat, E., and Benabid, A. L. (2009). Monophasic but not biphasic pulses induce brain tissue damage during monopolar high-frequency deep brain stimulation. Neurosurgery 64, 156-163.
Pittenger, C., and Duman, R. S. (2008). Stress, depression, and neuroplasticity: a convergence of mechanisms. Neuropsychopharmacology 33, 88-109.
Pizzagalli, D., Pascual-Marqui, R. D., Nitschke, J. B., Oakes, T. R., Larson, C. L., Abercrombie, H. C., Schaefer, S. M., Koger, J. V., Benca, R. M., and Davidson, R. J. (2001). Anterior cingulate activity as a predictor of degree of treatment response in major depression: evidence from brain electrical tomography analysis. Am. J. Psychiatry 158, 405-415.
Poole, J. L. (1954). Psychosurgery in older people. J. Am. Geriatr. Soc. 2, 456-466.
Post, R. M., Uhde, T. W., Roy-Byrne, P. P., and Joffe, R. T. (1986). Antidepressant effects of carbamazepine. Am. J. Psychiatry 143.29-34.
Quitkin, F. M., Rabkin, J. G., Stewart, J. W., McGrath, P. J., and Harrison, W. (1986). Study duration in antidepressant research: advantages of a 12-week trial. J. Psychiatr. Res. 20, 211-216.
Raisman, R., Sechter, D., Briley, M. S., Zarifan, E., and Langer, S. Z. (1981). High-affinity 3H-imipraminebinding in platelets from untreated and treated depressed patients compared to healthy volunteers. Psychopharmacology(Berl.) 75, 368-371.
Raison. C. L., Capuron. L., and Miller, A. H (2006). Cytokines sing the blues: inflammation and the pathogenesis of depression. Trends Immunol. 27, 24-31.
Robert LiKamWa, Yunxin Liu, Nicholas D. Lane and Lin Zhong entitled “MoodScope: Building a Mood Sensor from Smartphone Usage Patterns” published in MobiSys'13, June 25-28, 2013, Taipei, Taiwan.
Rubin, R. T., Poland, R. E., Lesser, I. M., Winston, R. A., and Blodgett, A. L. H (1987). Neuroendocrine aspects of primary endogenous depression. Cortisol secretory dynamics in patients and matched controls. Arch. Gen. Psychiatry 44, 328-336.
Ruhe, H. G., Mason, N. S., and Schene, A. H. (2007). Mood is indirectly related to serotonin, norepinephrine, and dopamine levels in humans: a meta-analysis of monoamine depletion studies. Mol. Psychiatry 12, 331-359.
Rush, A. J., George, M. S., Sackeim, H. A., Marangell, L. B., Mustafa, M. H., Giller, C., Nahas, Z., Haines, S., Simpson, R. K., Jr., and Goodman. R. (2000). Vagus Nerve Stimulation (VNS) for treatment resistant depressions: a multicenter study. Biol. Psychiatry 47, 276-286.
Sapolsky, R. M. (2000). Glucocorticoids and hippocampal atrophy in neu-ropsychiatric disorders. Arch. Gen. Psychiatry 57, 925-935.
Shimizu, E., Hashimoto, K., Okamura, N., Koike, K., Komatsu, N., Kumakiri, C., Nakazato, M., Watanabe, H., Shinoda, N., Okada, S., and Iyo, M. (2003). Alterations of serum levels of brain derived neurotrophic factor (BDNF) in depressed patients with or without antidepressants. Biol. Psychiatry 54, 70-75.
Smith, A. C., Shah, S. A., Hudson, A. E., Purpura, K. P., Victor, J. D., Brown, E. N., and Schiff, N. D. (2009). A bayesian statistical analysis of behavioral facilitation associated with deep brain stimulation. J. Neurosci. Methods 183, 267-276.
Smith, R. S. (1991). The macrophage theory of depression. Med. Hypotheses 35, 298-306.
Solomon, D. A., Keller, M. B., Leon, A. C., Mueller, T. I., Lavori, P. W., Shea, T., Coryell, W., Warshaw. M., Turvey, C., Maser, J. D., and Endicott, J. (2000). Multiple recurrences of major depressive disorder. Am. J. Psychiatry 157, 229-233.
Speer, A. M., Kimbrel, T. A., Wassermann, E. M., Repella, J. D., Willis, M. W., Herscovitch, P., and Post, R. M. (2000). Opposite effects of high and low frequency ifTMS on regional brain activity in depressed patients. Biol. Psychiatry 48, 1133-1141.
Sullivan. P. F., Neale, M. C., and Kendler, K. S. (2000). Genetic epidemiology of major depression: review and meta-analysis. Am. J. Psychiatry 157, 1552-1562.
Sun, F. T., Morrell. M. J., and Wharen, R. E. (2008). Responsive cortical stimulation for the treatment of epilepsy. Neurotherapeutics 5, 68-74.
Thase, M. E., and Rush, A. J. (1997). When at first you don't succeed: sequential strategies for antidepressant non-responders. J. Clin. Psychiatry 53, 649-659.
Tomarken, A. J., Davidson, R. J., Wheeler, R. E. and Doss, R. C. (1992). Individual differences in anterior brain asymmetry and fundamental dimensions of emotion. J. Pers. Soc. Psychol. 62, 676-687.
Tung, B., and Kleinrock, L. (1996). Using finite state automata to produce self-optimization and self-control. IEEE Trans. Parallel Distrib. Syst.7, 439-448.
Turner, E. H., Matthews, A. M., Linardatos, E., Tell, R. A., and Rosenthal, R. (2008). Selective publication of anti-depressant trials and its influence on apparent efficacy. N. Engl. J. Med. 358, 252-260.
Gerard D. van Rijsbergen, Claudi L. H. Bockting, Matthias Berking, Maarten W. J. Koeter and Aart H. Schene. “Can a One-Item Mood Scale Do the Trick?Predicting Relapse over 5.5-Years in Recurrent Depression.”, PLOS ONE 1 Oct. 2012 Vol 7 Issue 10.
Velasco, F., Velasco, M., Jimenez, F., Velasco A. L., and Salin-Pascual, R. (2005). Neurobiological background for performing surgical intervention in the inferior thalamic peduncle for treatment of major depression disorders. Neurosurgery 57, 439-448.
Wells, K. B., Stewart, A., Hays, R. D., Burnam, M. A., Rogers, W., Daniels. M., Berry. S., Greenfi ld, S., and Ware, J. (1989). The functioning and well being of depressed patients: results from the medical outcomes study. JAMA 262, 914-919.
Wong, P. K., Yu, F., Shahangian, A., Cheng, G., Sun, R., and Ho, C. M. (2008). Closed-loop control of cellular functions using combinatory drugs guided by a stochastic search algorithm. Proc. Natl. Acad. Sci. U.S.A. 105, 5105-5110.
Yirmiya, R., Pollak, Y., Morag, M., Reichenberg, A., Barak, O., Avitsur, R., Shavit, Y., Ovadia, H., Weidenfeld, J., Morag, A., Newman, M. E., and Pollmacher, T. (2000). Illness, cytokines, and depression. Ann. N. Y. Acad Sci. 917, 478-487.

What is claimed is:

1. A system for treating a mood disorder in a patient, the system comprising:
one or more implantable devices each device including one or more electrodes for sensing cortical signals in one or more cortical regions of the patient's brain and for stimulating one or more regions of the brain, one or more processor/controllers in communication with the one or more electrodes for receiving and processing sensed cortical signals to obtain cortical signals' processed data and for controlling the stimulating of one or more brain regions through the one or more electrodes;
at least one portable communication device operable by the patient and having an application software operating in the background, for automatically acquiring collected data representing objectively quantified parameters of use of the at least one portable communication device by the patient and for communicating the collected data to the one or more processor/controllers and/or to at least one remote processor,
wherein the collected data and the cortical signals' processed data are processed by the one or more processor/controllers, and/or by a processor included in the portable communication device and/or by the at least one remote processor to obtain a computed data value usable for initiating and/or modulating and/or controlling the stimulating of one or more brain regions to treat the mood disorder; and
at least one power source suitably electrically connected to the one or more implantable devices for providing power thereto.

2. The system according to claim 1, wherein the one or more implantable devices are selected from, one or more intra-cranially implantable devices, one or more implantable intra-calvarial devices, one or more devices implanted under a scalp of the patient and any combinations thereof.

3. The system according to claim 1, wherein the one or more electrodes are selected from, one or more intra-calvarial electrodes, one or more intra-calvarial electrode arrays, one or more intra-cranial electrodes, one or more intra-cranial electrode arrays, one or more electrodes implanted on or under a scalp of the patient, and any combinations thereof.

4. The system according to claim 1, wherein, at least one of the one or more implantable devices is an intra-calvarial device having intra-calvarial electrodes, disposed between an outer table and an inner table of the calvarial bone of the patient without fully penetrating the inner table of the calvarial bone.

5. The system according to claim 4, wherein, at least some of the electrodes of the intra-calvarial implant are in contact with an outer surface of the inner table of the calvarial bone.

6. The system according to claim 1, wherein the system includes one or more implantable frequency interference (FI) devices configured for stimulating one or more brain regions by using a frequency Interference stimulation method.

7. The system according to claim 6, wherein the one or more brain regions stimulatable by the implantable FI devices are selected from, at least one cortical region, at least one deep brain structure and any combinations thereof.

8. The system according to claim 7, wherein the at least one cortical region is selected from, the right dorsolateral prefrontal cortex (RDLPFC), the left dorsolateral prefrontal cortex (LDLPFC), one or more regions of the cingulate cortex, one or more regions of the prefrontal cortex (PFC) and any combinations thereof.

9. The system according to claim 7, wherein the at least one deep brain structure is selected from, ventral striatum (VS), one or more parts of the limbic system, a subgenual cingulate region (BA 25), a ventral capsule (VC), a nucleus accumbens, a lateral habenula, a ventral caudate nucleus, an inferior thalamic peduncle, an insula, and any combinations thereof.

10. The system according to claim 1, wherein the one or more cortical regions are selected from the right dorsolateral prefrontal cortex (RDLPFC), the left dorsolateral prefrontal cortex (LDLPFC), a region of the prefrontal cortex (PFC), and any combinations thereof.

11. The system according to claim 1, wherein the system also includes one or more sensor units in communication with the one or more processor/controllers for sensing one or more additional biomarkers indicative of the patient's mood.

12. The system according to claim 11, wherein the one or more sensor units are selected from, a temperature sensor, an accelerometer, a movement sensor, a heart rate sensor, a perspiration sensor, a pupillometry sensor, an eye tracking sensor, a microphone, a blood serotonin sensor, a blood dopamine sensor, a camera and any combination thereof.

13. The system according to claim 11, wherein the one or more biomarkers are selected from a change in one or more parameter selected from, a body temperature, a body acceleration, a body movement, a heart rate, a heart rate variability, a blood pressure, a perspiration rate, a pupil size in response to presentation of a negative word, an eye movement parameter, a vowel space of a patient's speech, blood serotonin level, blood dopamine level, and any combination thereof.

14. The system according to claim 11, wherein the system is capable of adjusting to changes in the patient's baseline physiology and/or patient's fundamental brain states over time by periodically monitoring patient's physiology using the sensed biomarkers and/or by periodically monitoring the patient's brain states using the collected data objectively quantified parameters of use of the at least one portable communication device by the patient, and/or the cortical signals' processed data.

15. The system according to claim 11, wherein at least one of the additional biomarkers indicative of the patient's mood comprises the patient's pupillary size changes responsive to a presentation of an image to the patient, wherein the system includes an augmented reality (AR) device operatively coupled to the one or more processor/controllers, and wherein the AR device is programmed to perform measurements of the patient's pupillary size changes responsive to a presentation of an image to the patient on the AR device.

16. The system according to claim 11, wherein at least one of the additional biomarkers indicative of the patient's mood comprises the patient's pupillary size changes responsive to a presentation of an image to the patient, and wherein the at least one portable communication device is used to perform pupillary size measurements and analysis to obtain pupillary size change data when an image is presented to the patient on a screen of the at least one portable communication device.

17. The system according to claim 16, wherein the measurements are performed during a time period selected from,
a test period during which one or more predetermined images are presented to the patient on the AR device,
a time period during which the patient browses web content using the AR device.

18. The system according to claim 1, wherein the mood disorder is selected from, major depressive disorder (MDD), post-traumatic stress disorder (PTSD), anxiety, treatment-resistant depression (TRD), and any combinations thereof.

19. The system according to claim 1, wherein the system also includes one or more effector devices controllable by the one or more processor/controllers and/or by the at least one portable communication device, the one or more effector device(s) are selected from, a device for delivering serotonin to the patient's brain, a device for delivering dopamine to the patient's brain and any combinations thereof.

20. The system according to claim 1, wherein the computed data value is a mood index MX and wherein the system is programmed to deliver stimulation to the one or more regions of the brain if the value of MX is smaller than or equal to a threshold level.

21. The system according to claim 20, wherein the value of MX is computed from data selected from,
the cortical signals' processed data and the collected data representing objectively quantified parameters of use of the at least one portable communication device by the patient,
the cortical signals' processed data, the collected data representing objectively quantified parameters of use of the at least one portable communication device by the patient, and one or more patient's biomarker data sensed by one or more sensors,
the cortical signals' processed data, the collected data representing objectively quantified parameters of use of the at least one portable communication device by the patient, and data obtained from voluntary responses of the patient to presentation of mood assessment requests presented on the at least one portable communication device
the cortical signals' processed data, the collected data representing objectively quantified parameters of use of the at least one portable communication device by the patient, data obtained from voluntary responses of the patient to presentation of mood assessment requests presented on the at least one portable communication device, and one or more patient's biomarker data sensed by one or more sensors.

22. The system according to claim 20, wherein the stimulation delivered to the one or more regions of the brain is graded stimulation.

23. The system according to claim 20, wherein the system also collects from the at least one portable communication device additional data selected from, data representing patient's movements, ambient light data, ambient sound data, patient's location data, patient's voice content, patient's texting content, patient sleep data, and wherein the system processes at least some of the additional data together with the collected data and the cortical signals' processed data to determine the value of MX.

24. The system according to claim 20, wherein the system includes a microphone, the microphone is selected from an external microphone operatively connected to the portable communication device and a microphone integrated in the portable communication device, and wherein the system is configured to perform voice spectral analysis of the patient's voice to obtain data representing a vowel production frequency range, wherein the data representing vowel production frequency range is used together with the collected data representing objectively quantified parameters of use of the at least one portable communication device by the patient, and/or the cortical signals' processed data in the computation of the value of MX.

25. The system according to claim 24, wherein the voice spectral analysis is automatically performed in the background using voice of the patient recorded during calls performed by the patient using the at least one portable communication device.

26. The system according to claim 20, wherein the system is a closed-loop neuromodulation system, that interrogates results of the stimulating of the one or more regions of the brain, using the computed value of MX to change one or more stimulation parameters responsive to changes in the value of MX.

27. The system according to claim 26, wherein the one or more stimulation parameters are selected from, stimulation amplitude, stimulating pulse width and stimulating pulse frequency and any non-mutually exclusive combinations thereof.

28. The system according to claim 1, wherein the at least one portable communication device is selected from, a mobile phone, a smartphone, a laptop, a mobile computer, a tablet, a notebook, a phablet, an augmented reality (AR) device, and any combinations thereof.

29. The system according to claim 1, wherein the one or more processor/controllers, and/or a processor included in the portable communication device and/or at least one remote processor are programmed to compute a mood index (MX) representing the momentary mood of the patient.

30. The system according to claim 29, wherein the value of MX is computed from data selected from,
the cortical signals' processed data, the collected data representing objectively quantified parameters of use of the at least one portable communication device by the patient, and data obtained from voluntary responses of the patient to presentation of mood assessment requests presented on the at least one portable communication device, and
the cortical signals' processed data, the collected data representing objectively quantified parameters of use of the at least one portable communication device by the patient, data obtained from voluntary responses of the patient to presentation of mood assessment requests presented on the at least one portable communication device, and one or more patient's biomarker data sensed by one or more sensors,
and wherein the patient voluntary responses are selected from,
responses assessing mood based on a two-dimensional construct comprising different levels of valence and arousal, and
responses assessing mood based on a one-dimensional construct.

31. The system according to claim 1, wherein the one or more processor/controllers or a processor/controller included in the at least one communication device or a remote processor are programmed to deliver stimulation to the one or more regions of the brain if the value of MX is smaller than or equal to a threshold level.

32. The system according to claim 1, wherein one or more of the collected data representing objectively quantified parameters of use of the at least one portable communication device by the patient, and the cortical signals' processed data, are used to periodically assess the effects of the stimulating on the mood and/or physiology of the patient.

33. The system according to claim 1, wherein the collected data representing objectively quantified parameters of use of the at least one portable communication device by the patient also comprises data obtained by automatically monitoring of the patient's pupillary size variations responsive to a presentation of words with negative emotional content.

34. The system according to claim 1, wherein the collected data representing objectively quantified parameters of use of the at least one portable communication device by the patient, also comprises automatically obtained data representing multiple parameters collected from the at least one portable communication device.

35. The system according to claim 34, wherein the collected data representing objectively quantified parameters of use of the at least one portable communication device by the patient, comprises data selected from one or more of, data representing application use by the patient, data representing number of calls made by the patient, acceleration data due to patient's movements, communication data, ambient light data, ambient sound data, patient's location data, patient's call log, patient's voice content, patient's texting content, patient sleep data, patient's social network data, and any combinations thereof.

36. The system according claim 1, wherein the cortical signals' processed data comprises a modulation index (MI) selected from,
a modulation index computed from the spectral power in one or more spectral bands of the cortical signals' processed data, and
a modulation index computed from the phase-amplitude coupling (PAC) in the cortical signals' processed data.

37. The system according to claim 1, wherein the collected data representing objectively quantified parameters of use of the at least one portable communication device by the patient, comprises data selected from one or more of, data representing use of one or more software applications of the portable communication device by the patient, data representing a number or frequency of calls made by the patient using the portable communication device, data representing a number or frequency of SMS text messages sent by the patient using the portable communication device, data representing the duration of phone calls made by the patient using the portable communication device, data representing the total number of times the screen of the portable communication device is turned on per day, data representing the total amount of patient's screen time per day, data representing acceleration of the portable communication device, data representing the frequency of use and/or the total time of use of one or more software applications installed on the portable communication device, data representing the frequency of use and/or the total time of use of the one or more software applications categorized by application category, data representing the number of photos taken by the patient per day using the portable communication device, patient's call log data, patient's social network data, and any combinations thereof.

38. The system according to claim 1, wherein the portable communication device is a mobile phone or a smartphone and wherein the collected data representing objectively quantified parameters of use of the at least one portable communication device by the patient, comprises data selected from one or more of, data representing use of one or more smartphone or mobile phone applications by the patient, data representing a number or frequency of phone calls made by the patient, data representing a number or frequency of SMS text messages sent by the patient, data representing the duration of phone calls made by the patient, data representing the total number of times the screen of the phone is turned on per day, data representing the total amount of patient's screen time per day, data representing the frequency of use and/or the total time of use of one or more phone applications, data representing the frequency of use and/or the total time of use of one or more phone applications categorized by application category, data representing the number of photos taken by the patient per day using the phone, patient's call-log data, patient's social network data, and any combinations thereof.

39. The system according to claim 38, wherein the data representing use of one or more software applications of the portable communication device by the patient, the data representing the frequency of use and/or the total time of use of one or more software applications installed on the portable communication device and the data representing the frequency of use and/or the total time of use of the one or more software applications categorized by application category, comprise data from the top five software applications used by the patient.

40. The system according to claim 38, wherein the data representing a number or frequency of calls made by the patient using the portable communication device, the data representing a number or frequency of SMS text messages sent by the patient using the portable communication device, and the data representing the duration of phone calls made by the patient using the portable communication device, comprise data of the top five contacts of the patient.

41. The system according to claim 38 wherein at least some of the collected data representing objectively quantified parameters of use of the at least one portable communication device by the patient, is normalized for each individual patient.

42. The system according to claim 41, wherein the AR device is programmed to periodically record the patient's pupillary size changes responsive to a presentation of an image to the patient on the AR device.

43. The system according to claim 41, wherein the AR device has eye-tracking capabilities and wherein the AR device is programmed to record pupillary size change data when the patient views an image presented on the AR device by using the eye tracking capability to detect an image currently viewed by the patient and by recording pupillary size of the patient before, during and after viewing the image, detecting the classification of the image using an LUT including images classified as images having a neutral emotional connotation and images having a negative emotional connotation, wherein the system is programed to perform an analysis of the pupillary size change data to assess the mood of the patient.

44. The system according to claim 43, wherein the analysis of the pupillary size change data includes data representing one or more parameters selected from,
an amplitude of a late pupil dilation in response to the presentation of the image,
a latency of the pupil dilation in response to the presentation of the image, and
a duration of the pupil dilation in response to the presentation of the image.

45. The system according to claim 43, wherein the image is an image representing a word.

46. The system according to claim 45, wherein the analysis of the pupillary size change data includes data representing one or more parameters selected from,
an amplitude of a late pupil dilation in response to the presentation of the image,
a latency of the pupil dilation in response to the presentation of the image, and
a duration of the pupil dilation in response to the presentation of the image.

47. The system according to claim 46, wherein the image is an image of a word.

48. The system according to claim 46, wherein the measurements are performed during a time period selected from,
a test period during which one or more predetermined images are presented to the patient on the screen of the portable communication device,
a time period during which the patient browses web content on the screen of the portable communication device.

49. A method for treating a mood disorder of a patient comprising:
receiving cortical electrical signals sensed in one or more cortical regions of the brain of the patient by one or more electrodes;
automatically receiving from at least one portable communication device usable by the patient objectively quantified data representing the parameters of use of the at least one communication device by the patient, the at least one communication device has an application software operating in the background for automatically acquiring the objectively quantified data or to receive the objectively quantified data from a remote processor in communication with the at least one communication device;
processing the cortical signals and the objectively quantified data to detect an indication that the patient is in a depressed mood requiring therapeutic stimulation; and
electrically stimulating at least one brain region of the patient responsive to detecting the indication.

50. The method according to claim 49, wherein the cortical electrical signals of the step of receiving are recorded by one or more implants selected from, extra-cranial implants, intra-cranial implants, intra-calvarial implants, implants disposed under the scalp of the patient, implants disposed between the scalp and the outer surface of the outer table of the calvarial bone of the patient, and any combinations thereof.

51. The method according to claim 49, wherein the cortical signals of the step of receiving are recorded by one or more electrodes selected from,
one or more intra-calvarial electrodes,
one or more intra-calvarial electrode arrays,
one or more intra-cranial electrodes,
one or more intra-cranial electrode arrays,
one or more electrodes implanted under the scalp of the patient,
one or more electrode arrays implanted under the scalp of the patient,
one or more intra-calvarial electrodes disposed in contact with or adjacent to an outer surface of the inner table of the calvarial bone,
one or more intra-calvarial electrode arrays disposed in contact with or adjacent to an outer surface of the inner table of the calvarial bone,
one or more intra-calvarial electrodes disposed between an inner table and an outer table of the calvarial bone, and
any combinations thereof.

52. The method according to claim 49, wherein the step of processing comprises processing the cortical signals and/or the objectively quantified data to select an electrical therapeutic paradigm for the patient and wherein the step of stimulating includes delivering therapeutic electrical signals to the at least one brain region of the patient according to the selected stimulation paradigm.

53. The method according to claim 49, wherein the objectively quantified data includes data selected from, data representing application use by the patient, data representing number of calls made by the patient, acceleration data due to patient's movements, communication data, ambient light data, ambient sound data, patient's location data, patient's call log, patient's voice content, patient's texting content, patient sleep data, patient's social network data, and any combinations thereof.

54. The method according to claim 49, wherein the step of processing includes processing the electrical cortical signals and the objectively quantified data to compute a value of a modulation index parameter MI and/or to compute a value of a mood index MX of the patient.

55. The method according to claim 49, wherein the step of automatically receiving also includes the step of automatically receiving one or more biomarker data of the patient from one or more sensors, and wherein the step of processing comprises processing the cortical signals, the objectively quantified data and the biomarker data to detect an indication that the patient is in a depressed mood requiring therapeutic stimulation.

56. The method according to claim 55, wherein the step of processing includes processing the electrical cortical signals, the objectively quantified data and the biomarker data to compute a value of a modulation index parameter MI and/or to compute a mood index MX of the patient.

57. The method according to claim 54, wherein the step of processing includes processing the cortical signals by computing the spectral power in one or more spectral bands and computing from the spectral power the modulation index MI.

58. The method according to claim 54, wherein the step of processing includes comparing the value of MI to a threshold value, and wherein the step of stimulating comprises stimulating the at least one brain region if the value of MI is equal to or larger than the threshold value.

59. The method according to claim 54, wherein the step of processing includes comparing the value of the mood index MX to a threshold value, and wherein the step of stimulating comprises stimulating one or more brain regions if the value of MX is equal to or larger than the threshold value.

60. The method according to claim 49, wherein the step of stimulating includes stimulating one or more brain regions, selected from one or more cortical brain regions, one or more deep brain structure and any combinations thereof.

61. The method according to claim 49, wherein the step of receiving comprises receiving cortical signals from one or more cortical regions selected from a right DLPFC, a left DLPFC, a region of the PFC and any combinations thereof.

62. The method according to claim 49, wherein the method also includes the step of using the at least one portable communication device to receive from the patient subjective mood self-assessment data, and wherein the step of processing includes processing the patient's subjective mood self-assessment data, the objectively quantified data and the cortical electrical signals to detect an indication that the patient is in a depressed mood requiring therapeutic stimulation to detect an indication that the patient is in a depressed mood requiring therapeutic stimulation.

63. The method according to claim 49, wherein the method also includes the step of interacting with the patient through the at least one portable communication device to receive voluntary patient input representing the patient's subjective momentary mood self-assessment, and wherein the step of processing includes processing the objectively quantified data, the patient's subjective mood self-assessment and the cortical electrical signals to detect the indication that the patient is in a depressed mood requiring therapeutic stimulation.

64. The method according to claim 63, wherein the step of interacting is automatically performed periodically.

65. The method according to claim 63, wherein the method also includes the step of interacting with the patient through the at least one portable communication device to receive voluntary patient input representing a subjective mood self-assessment data, and wherein the step of processing includes processing the subjective mood assessment, the objectively quantified data and the electrical cortical signals to select and/or modify a therapeutic stimulation paradigm to be delivered the patient.

66. The method according to claim 65, wherein the step of receiving also includes receiving from the patient subjective voluntary mood self-assessment data in response to a step of the at least one communication device prompting the patient to provide subjective voluntary mood self-assessment data.

67. The method according to claim 49, wherein the one or more cortical brain regions of the step of stimulating are selected from a right DLPFC, a left DLPFC, a region of the PFC, a subgenual cingulated cortex, and any combinations thereof, and wherein the one or more deep brain structures of the step of stimulating are selected from a ventral striatum (VS), one or more parts of the limbic system, a subgenual cingulate region (BA 25), a ventral capsule (VC), a nucleus accumbens, a lateral habenula, a ventral caudate nucleus, an inferior thalamic peduncle, an insula, and any combinations thereof.

68. The method according to claim 49, wherein the mood disorder is selected from, major depressive disorder (MDD), treatment-resistant depression (TRD), post-traumatic stress disorder (PTSD), anxiety, and any combinations thereof.

69. The method according to claim 49, wherein the at least one portable communication device is selected from, a mobile phone, a smartphone, a laptop, a mobile computer, a tablet, a notebook, a phablet, an augmented reality (AR) device and any combinations thereof.

70. The method according to claim 54, wherein the step of processing includes computing the value of the modulation index MI from the cortical signals, computing the value of the mood index (MX) from the value of MI and the objectively quantified data, and wherein the step of stimulating comprises stimulating the at least one brain region if the value of MX is equal to or larger than a threshold value.

71. A method for treating a mood disorder of a patient using the system of claim 1, the method comprising:
receiving cortical electrical signals sensed in one or more cortical regions of the brain of the patient by the one or more electrodes of the one or more implants;
automatically receiving from the at least one portable communication device objectively quantified data representing the parameters of use of the at least one communication device by the patient, the at least one communication device has an application software operating in the background for automatically acquiring the objectively quantified data or to receive the objectively quantified data from a remote processor in communication with the at least one communication device;
processing the cortical signals and the objectively quantified data to detect an indication that the patient is in a depressed mood requiring therapeutic stimulation; and
using the one or more electrodes to electrically stimulate at least one brain region of the patient responsive to detecting the indication.
